(19) United States
(12) Patent Application Publication
(10) Pub. No.: US 2003/0105277 A1
(43) Pub. Date: Jun. 5, 2003

(54) Compositions and therapeutic methods for viral infection

(75) Inventors: Scott Morham, Salt Lake City, UT (US); Kenton Zavitz, Salt Lake City, UT (US); Adrian Hobden, Salt Lake City, UT (US)

Correspondence Address: MYRIAD GENETICS INC., LEGAL DEPARTMENT, 320 WAKARA WAY, SALT LAKE CITY, UT, 84108, US

(73) Assignee: Myriad Genetics, Incorporated, Salt Lake City, UT

(21) Appl. No.: 10/226,007

(22) Filed: Aug. 21, 2002

Related U.S. Application Data
(60) Provisional application No. 60/313,883, filed on Aug. 21, 2001.

(51) Int. Cl. A61K 39/12; C07K 14/005; C07K 14/15; A61K 38/00; C07K 1/00; C07K 14/00; C07K 17/00; C07K 2/00; C07K 4/00; C07K 5/00; C07K 7/00; C07K 16/00

(52) U.S. Cl. 530/300; 514/12; 424/186.1; 530/350

(57) ABSTRACT

Methods for inhibiting viral propagation and treating viral infection are provided which include administering to cells infected with viruses a compound capable of inhibiting viral budding from the infected host cells.

### RELATED U.S. APPLICATIONS

This application claims priority under 35 U.S.C. §119(e) to U.S. Provisional Application Serial No. 60/313,883 filed on Aug. 21, 2001, which is incorporated herein by reference in its entirety.

### FIELD OF THE INVENTION

The present invention generally relates to pharmaceuticals and methods of treating diseases, particularly to methods and pharmaceutical compositions for treating viral infections.

### BACKGROUND OF THE INVENTION

Viruses are the smallest of parasites, and are completely dependent on the cells they infect for their reproduction. Viruses are composed of an outer coat of protein, which is sometimes surrounded by a lipid envelope, and an inner nucleic acid core consisting of either RNA or DNA. Generally, after docking with the plasma membrane of a susceptible cell, the viral core penetrates the cell membrane to initiate the viral infection. After infecting cells, viruses commandeer the cell's molecular machinery to direct their own replication and packaging. The “replicative phase” of the viral lifecycle may begin immediately upon entry into the cell, or may occur after a period of dormancy or latency. After the infected cell synthesizes sufficient amounts of viral components, the “packaging phase” of the viral life cycle begins and new viral particles are assembled. Some viruses reproduce without killing their host cells, and many of these bud from host cell membranes. Other viruses cause their host cells to lyse or burst, releasing the newly assembled viral particles into the surrounding environment, where they can begin the next round of their infectious cycle.

Several hundred different types of viruses are known to infect humans, however, since many of these have only recently been recognized, their clinical significance is not fully understood. Of these viruses that infect humans, many infect their hosts without producing overt symptoms, while others (e.g., influenza) produce a well-characterized set of symptoms. Importantly, although symptoms can vary with the virulence of the infecting strain, identical viral strains can have drastically different effects depending upon the health and immune response of the host. Despite remarkable achievements in the development of vaccines for certain viral infections (i.e., polio and measles), and the eradication of specific viruses from the human population (e.g., smallpox), viral diseases remain as important medical and public health problems. Indeed, viruses are responsible for several “emerging” (or reemerging) diseases (e.g., West Nile encephalitis & Dengue fever), and also for the largest pandemic in the history of mankind (HIV and AIDS).

Viruses that primarily infect humans are spread mainly via respiratory and enteric excretions. These viruses are found worldwide, but their spread is limited by inborn resistance, prior immunizing infections or vaccines, sanitary and other public health control measures, and prophylactic antiviral drugs. Zoonotic viruses pursue their biologic cycles chiefly in animals, and humans are secondary or accidental hosts. These viruses are limited to areas and environments able to support their nonhuman natural cycles of infection (vertebrates or arthropods or both). However, with increased global travel by humans, and the likely accidental co-transport of arthropod vectors bearing viral payloads, many zoonotic viruses are appearing in new areas and environments as emerging diseases. For example, West Nile virus, which is spread by the bite of an infected mosquito, and can infect people, horses, many types of birds, and other animals, was first isolated from a febrile adult woman in the West Nile District of Uganda in 1937. The virus made its first appearance in the Western Hemisphere, in the New York City area in the autumn of 1999, and during its first year in North America, caused the deaths of 7 people and the hospitalization of 62. At the time of this writing (August, 2002) the virus has been detected in birds in 37 states and the District of Columbia, and confirmed human infections have occurred in Alabama, the District of Columbia, Florida, Illinois, Indiana, Louisiana, Massachusetts, Mississippi, Missouri, New York City, Ohio, and Texas. (See: http://www.cdc.gov/od/oc/media/wncount.htm).

Additionally, some viruses are known to have oncogenic properties. Human T-cell lymphotropic virus type 1 (a retrovirus) is associated with human leukemia and lymphoma. Epstein-Barr virus has been associated with malignancies such as nasopharyngeal carcinoma, Burkitt's lymphoma, Hodgkin's disease, and lymphomas in immunosuppressed organ transplant recipients. Kaposi's sarcoma-associated virus is associated with Kaposi's sarcoma, primary effusion lymphomas, and Castleman's disease (a lymphoproliferative disorder).

Treatment of viral diseases presents unique challenges to modern medicine. Since viruses depend on host cells to provide many functions necessary for their multiplication, it is difficult to inhibit viral replication without at the same time affecting the host cell itself. Consequently, antiviral treatments are often directed at the functions of specific enzymes of particular viruses. However, such antiviral treatments that specifically target viral enzymes (e.g., HIV protease, or HIV reverse transcriptase) often have limited usefulness, because resistant strains of viruses readily arise through genetic drift and mutation.

### SUMMARY OF THE INVENTION

The present invention provides a method for inhibiting viral budding from virus-infected cells and thus inhibiting virus propagation in the cells. The method includes administering to the cells a compound comprising an amino acid sequence motif of PX₁X₂X₃ and capable of binding a type I WW-domain of the cellular protein Nedd4 (neuronal precursor cell expressed developmentally downregulated 4), wherein X₃ is Y or W or an analog thereof. The method is useful in the treatment of viral infections caused by viruses that utilize the Nedd4 protein or a Nedd4-like protein of their host cells for viral budding within and/or out of infected cells. The method can be used in treating virus infection caused by viruses such as hepatitis B virus, hepatitis E virus, human herpesviruses, Epstein-Barr virus, polyomavirus, Marburg virus, TT virus, lassa virus, lymphocytic choriomeningitis virus, vesicular stomatitis virus, and infectious pancreatic necrosis virus. In particular, the method is useful in the treatment of viral infections caused either hepatitis B virus or human herpesvirus 1. In addition, the method can also be useful in treating and preventing symptoms caused by and/or associated to viral infection.

In a first aspect of the invention, a method for treating viral infection is provided, which comprises administering to a patient in need of such treatment a composition comprising a peptide having an amino acid sequence motif PPXY, wherein X is an amino acid, and the peptide and is capable of binding a type I WW-domain of the Nedd4 protein. In preferred embodiments, X is proline (P), alanine (A), glutamic acid (E), asparagine (N), or arginine (R). Preferably, the peptide consists of from about 8 to about 100 amino acid residues, more preferably from 9 to about 50, or from 10 to about 20 amino acid residues.

In specific embodiments, the peptide includes a contiguous amino acid sequence of at least 6, preferably at least 8 amino acid residues, and more preferably from about 8 to about 30 or from about 9 to 20 amino acid residues of a viral protein selected from the group consisting of matrix proteins of rhabdoviruses, matrix proteins of filoviruses, Rous Sarcoma virus GAG protein, Mason-Pfizer Monkey virus GAG protein, hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, TT virus ORF2 protein; wherein said contiguous amino acid sequence encompasses the PPXY motif of the viral protein. Alternatively, the peptide includes a contiguous amino acid sequence of at least 6 amino acid residues of a viral protein selected from the group consisting of Ebola virus Matrix (EbVp40) protein, Marburg virus matrix protein, VSV matrix protein, and Mason-Pfizer Monkey virus GAG protein, and wherein said contiguous amino acid sequence encompasses the PPXY motif of said viral protein, wherein the peptide is capable of binding a type I WW-domain of Nedd4. For example, the peptide in the hybrid poly peptide can include an amino acid sequence selected from the group consisting of SEQ ID NOs:24-36, SEQ ID NOs:154-295, SEQ ID NOs:296-438, SEQ ID NOs:439-581, SEQ ID NOs:582-724, SEQ ID NOs:725-1010, SEQ ID NOs:1011-1296, SEQ ID NOs:1297-1439, SEQ ID NOs:1440-1452, SEQ ID NOs:1453-1491, SEQ ID NOs:1492-1530, and SEQ ID NOs:1531-1673.

In a specific embodiment, the peptide does not include a contiguous amino acid sequence of Ebola virus Matrix (EbVp40) protein that is sufficient to impart an ability to bind the UEV domain of the human Tsg101 protein.

In preferred embodiments, the peptide in the composition is associated with, or more preferably covalently linked to, a transporter that is capable of increasing the uptake of the peptide by a mammalian cell. In highly preferred embodiments the transporter increases uptake by at least 100%, preferably at least 300%. Advantageously, the transporter is selected from the group consisting of penetrating, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histidine oligomers, D-histidine oligomers, L-ornithine oligomers, D-ornithine oligomers, and HSV-1 structural protein VP22 and fragments thereof, and peptides having at least six contiguous amino acid residues that are L-arginine, D-arginine, L-lysine, D-lysine, L-histidine, D-histidine, L-ornithine, D-ornithine, or a combination thereof; and peptoid analogs thereof. Alternatively, the transporter can be non-peptidic molecules or structures such as liposomes, dendrimers, and siderophores.

When a transporter covalently linked to a peptide of the present invention is peptidic transporter, a hybrid polypeptide is provided. In one embodiment, the hybrid polypeptide consists of from about 8 to about 100 amino acid residues, preferably from about 9 to about 50 amino acid residues. In preferred embodiments, the hybrid polypeptide consists of from about 12 to about 30 amino acid residues. In specific embodiments, X is either a proline (P), alanine (A), glutamic acid (E), asparagine (N), or an arginine (R).

Advantageously, the peptidic transporter in the hybrid polypeptide is capable of increasing the uptake of the peptide by a mammalian cell by at least 100%, preferably at least 300%. Examples of the peptidic transporter include penetrating, l-Tat₄₉₋₅₇, retro-inverso isomers of l-Tat₄₉₋₅₇, L-arginine oligomers, L-lysine oligomers, HSV-1 structural protein VP22 and fragments thereof, and peptides consisting of at least six contiguous amino acid residues that include two or more of the group consisting of L-arginine, L-lysine and L-histidine. However, in certain embodiments, the hybrid polypeptide does not contain a terminal L-histidine oligomer.

Various modifications may be made to improve the stability and solubility of the compound, and/or optimize its binding affinity to Nedd4, particularly to a type I WW domain of Nedd4. In particular, various protection groups can be incorporated into the amino acid residues of the compounds. In addition, the compounds according to the present invention can also be in various pharmaceutically acceptable salt forms.

The foregoing and other advantages and features of the invention, and the manner in which the same are accomplished, will become more readily apparent upon consideration of the following detailed description of the invention taken in conjunction with the accompanying examples, which illustrate preferred and exemplary embodiments.

### DETAILED DESCRIPTION OF THE INVENTION

As used herein, the term “viral infection” generally encompasses infection of an animal host, particularly a human host, by one or more viruses. Thus, treating viral infection will encompass the treatment of a person who is a carrier of one or more specific viruses or a person who is diagnosed of active symptoms caused by and/or associated with infection by the viruses. A carrier of virus may be identified by any methods known in the art. For example, a person can be identified as virus carrier on the basis that the person is antiviral antibody positive, or is virus-positive, or has symptoms of viral infection. That is, “treating viral infection” should be understood as treating a patient who is at any one of the several stages of viral infection progression. In addition, “treating or preventing viral infection” will also encompass treating suspected infection by a particular virus after suspected past exposure to virus by e.g., blood transfusion, exchange of body fluids, bites, accidental needle stick, or exposure to patient blood during surgery, or other contacts with a person with viral infection that may result in transmission of the virus.

Specifically, as used herein, the term “HBV infection” generally encompasses infection of a human by any strain or serotype of hepatitis B virus, including acute hepatitis B infection and chronic hepatitis B infection. Thus, treating HBV infection means the treatment of a person who is a carrier of any strain or serotype of hepatitis B virus or a person who is diagnosed of active hepatitis B to reduce the HBV viral load in the person or to alleviate one or more symptoms associated with HBV infection and/or hepatitis B, including, e.g., nausea and vomiting, loss of appetite, fatigue, muscle and joint aches, elevated transaminase blood levels, increased prothrombin time, jaundice (yellow discoloration of the eyes and body) and dark urine. A carrier of HBV may be identified by any methods known in the art. For example, a person can be identified as HBV carrier on the basis that the person is anti-HBV antibody positive (e.g., based on hepatitis B core antibody or hepatitis B surface antibody), or is HBV-positive (e.g., based on hepatitis B surface antigen or HBV RNA or DNA) or has symptoms of hepatitis B infection or hepatitis B. That is, “treating HBV infection” should be understood as treating a patient who is at any one of the several stages of HBV infection progression. In addition, the term “treating HBV infection” will also encompass treating suspected infection by HBV after suspected past exposure to HBV by, e.g., contact with HBV-contaminated blood, blood transfusion, exchange of body fluids, “unsafe” sex with an infected person, accidental needle stick, receiving a tattoo or acupuncture with contaminated instruments, or transmission of the virus from a mother to a baby during pregnancy, delivery or shortly thereafter. The term “treating HBV infection” will also encompass treating a person who is free of HBV infection but is believed to be at risk of infection by HBV.

The term “preventing hepatitis B” as used herein means preventing in a patient who has HBV infection or is suspected to have HBV infection or is at risk of HBV infection from developing hepatitis B (which is characterized by more serious hepatitis-defining symptoms).

The terms “polypeptide,” “protein,” and “peptide” are used herein interchangeably to refer to amino acid chains in which the amino acid residues are linked by peptide bonds or modified peptide bonds. The amino acid chains can be of any length of greater than two amino acids. Unless otherwise specified, the terms “polypeptide,” “protein,” and “peptide” also encompass various modified forms thereof. Such modified forms may be naturally occurring modified forms or chemically modified forms. Examples of modified forms include, but are not limited to, glycosylated forms, phosphorylated forms, myristoylated forms, palmitoylated forms, ribosylated forms, acetylated forms, etc. Modified forms also encompass pharmaceutically acceptable salt forms. In addition, modifications also include intra-molecular crosslinking and covalent attachment to various moieties such as lipids, flavin, biotin, polyethylene glycol or derivatives thereof, etc. In addition, modifications may also include cyclization, and branching. Further, amino acids other than the conventional twenty amino acids encoded by genes may also be included in a polypeptide.

As used herein, the term “Nedd4” means human Nedd4 protein, unless otherwise specified.

The recruitment of cellular machinery to facilitate viral budding appears to be a general phenomenon, and distinct late domains have been identified in the structural proteins of several other enveloped viruses. See Vogt, Proc. Natl. Acad. Sci. USA, 97:12945-12947 (2000). Two well characterized late domains are the “PY” motif (consensus sequence: PPXY; X=any amino acid) found in membrane-associated proteins from certain enveloped viruses. See Craven et al., J. Virol., 73:3359-3365 (1999); Harty et al., Proc. Natl. Acad. Sci. USA, 97:13871-13876 (2000); Harty et al., J. Virol., 73:2921-2929 (1999); and Jayakar et al., J. Virol., 74:9818-9827 (2000). The cellular target for the PY motif is Nedd4, which also contains a Hect ubiquitin E3 ligase domain. The “YL” motif (YXXL) was found in the Gag protein of equine infectious anemia virus (EIAV). Puffer et al., J. Virol., 71:6541-6546 (1997); Puffer et al., J. Virol., 72:10218-10221 (1998). The cellular receptor for the “YL” motif appears to be the AP-50 subunit of AP-2. Puffer et al., J. Virol., 72:10218-10221 (1998). Interestingly, the late domains such as the P(T/S)AP motif, PY motif and the YL motif can still function when moved to different positions within retroviral Gag proteins, which suggests that they are docking sites for cellular factors rather than structural elements. Parent et al., J. Virol., 69:5455-5460 (1995); Yuan et al., EMBO J., 18:4700-4710 (2000). Moreover, the late domains such as the P(T/S)AP motif, PY motif and the YL motif can function interchangeably. That is one late domain motif can be used in place of another late domain motif without affecting viral budding. Parent et al., J. Virol., 69:5455-5460 (1995); Yuan et al., EMBO J., 18:4700-4710 (2000); Strack et al., Proc. Natl. Acad. Sci. USA, 97:13063-13068 (2000).

Nedd4 is a ubiquitin protein ligase containing a ubiquitin ligase Hect domain and several so-called WW domains. Specifically, the second and third WW-domains of Nedd4 are Type I WW-domains, which are found to bind to the PY motifs of a few viruses. The Hect ubiquitin E3 ligase domain transfers ubiquitin onto specific protein substrates and can “mark” surface receptors for endocytosis by monoubiquitination. See Harvey and Kumar, Trends Cell Biol., 9:166-169 (1999); Hicke, Trends Cell Biol., 9:107-112 (1999). The PY motif binds Nedd4 via one or more of the type I WW-domains in Nedd4. See Kanelis et al., Nat. Struct. Biol., 8:407-412 (2001); Lu et al., Science, 283:1325-1328 (1999).

Accordingly, while not wishing to be bound by any theory, it is believed that although the three late domain motifs bind to different cellular targets, they utilize common cellular pathways to effect viral budding. In particular, it is believed that the different cellular receptors for viral late domain motifs feed into common downstream steps of the vacuolar protein sorting (VPS) and MVB pathway. As is known in the art, all three cellular targets, i.e., Tsg101, Nedd4 and AP-2, function in the VPS pathway. Another protein, Vps4, functions in Tsg101 cycling and endosomal trafficking. Particularly, Vps4 mutants prevent normal Tsg101 trafficking and induce formation of aberrant, highly vacuolated endosomes that are defective in the sorting and recycling of endocytosed substrates. See Babst et al, Traffic, 1:248-258 (2000); Bishop and Woodman, J. Biol. Chem., 276:11735 (2001).

While not wishing to be bound by any theory, it is believed that the PY motif or a variation thereof enables a protein containing the PY motif to bind the cellular protein Nedd4, and that the binding of the PY motif in viral proteins to a type I WW-domain of Nedd4 or another cellular protein (e.g., a Nedd4-like cellular protein) enables viruses having the PY motif to usurp cellular machinery normally used for MVB formation to allow viral budding from the plasma membrane. Nedd4 and/or other Nedd4-like proteins may serve as the common docking site for all viruses that utilize the PY motif to bud off host cell cytoplasm membrane. It is also believed that depletion of Nedd4 or other Nedd4-like proteins or interfering with the interaction between Nedd4 (and/or other Nedd4-like proteins) and the PY motif in virus-infected cells will prevent viral budding from the cells.

In accordance with the present invention, a number of viral proteins have been found to also contain the PY motif. The proteins are summarized in Table 1 below.

TABLE 1
Viral Proteins Containing the P Y Motif

| | PPPY- | GenBank | |
|---|---|---|---|
| | Containing | Accession | SEQ ID |
| Virus | Protein | No. | NO: |
| Ebola Virus | Matrix Protein | AAL25816 | 27 |
| Marburg Virus | VP40 Protein | NP_042027 | 28 |
| Vesicular Stomatitis | Matrix Protein | P04876 | 29 |
| Virus | | | |
| Rous Sarcoma Virus | GAG Protein | AAA19608 | 30 |
| Hepatitis B Virus | | | |
| (Isolate Patient Usai ′89) | Core Antigen | S53155 | 31 |
| Human Herpesvirus 4 | Latent Membrane | CAA57375 | 32 |
| (Epstein-Barr Virus) | Protein 2A | | |
| Human Herpesvirus 1 | UL56 Protein | A43965 | 33 |
| (Strain F) | | | |
| Human Herpesvirus 7 | Major Capsid | AAC40768 | 34 |
| | Scaffold Protein | | |
| Infectious Pancreatic | Structural Protein | AAK18736 | 35 |
| Necrosis Virus | VP2 | | |
| Lassa Virus | Z Protein | AAC05816 | 36 |
| Lymphocytic | Ring Finger Protein | CAA10342 | 37 |
| Choriomeningitis Virus | | | |
| TT Virus | ORF2 | BAB19319 | 38 |

The inventors therefore propose using peptides containing a PY motif and capable of binding a type I WW-domain of Nedd4 or a Nedd4-like protein in treating viral infection, particularly infections caused by viruses that utilizes their PY motif in viral budding.

Thus, in accordance with a first aspect of the present invention, a method is provided for inhibiting viral budding from virus-infected cells and thus inhibiting virus propagation in the cells. The method includes administering to the cells a compound capable of binding to one or more type I WW-domains of Nedd4 or a Nedd4-like protein (e.g., E3 ubiquitin ligase).

Specifically, the method comprises administering to the cells a compound having an amino acid sequence motif of PX₁X₂X₃, wherein X₃ is Y or W or an analog thereof. In one embodiment, the X₁ in the motif is P or an analog thereof. In a preferred embodiment, the compound administered has the amino acid sequence motif of PX₁X₂X₃, wherein X₁ is P or an analog thereof, and X₃ is Y or W or an analog thereof. In a more preferred embodiment, X₁ in the PX₁X₂X₃ motif is P or an analog thereof, and X₂ is P or an analog thereof, and X₃ is Y or W or an analog thereof. In a most preferred embodiment, X₁ in the PX₁X₂X₃ motif is P or an analog thereof, and X₂ is P or an analog thereof, and X₃ is Y or an analog thereof. In preferred embodiments, the compounds are capable of binding a WW domain of Nedd4 or a Nedd4-like protein of a human cell. The compounds can be administered to cells in vitro or cells in vivo in a human or animal body. In the case of in vivo applications of the method, viral infection can be treated and alleviated by using the compound to inhibit virus propagation.

In preferred embodiments, the method comprises administering to cells a composition comprising a peptide having an amino acid sequence motif PPXY and capable of binding a type I WW-domain of the Nedd4 protein, wherein X is an amino acid.

The method of the present invention can be used for inhibiting viral budding by an enveloped virus. Advantageously, the method is used for inhibiting viral budding by viruses such as rhabdoviruses (e.g., vesicular stomatitis virus), filoviruses (e.g., Ebola virus and Marburg virus), Rous Sarcoma virus, hepatitis B virus (“HBV”), human herpesvirus 1 (HSV1), human herpesvirus 4 (HSV4), human herpesvirus 7 (HSV7), infectious pancreatic necrosis virus, Lassa virus, lymphocytic choriomeningitis virus, Epstein-Barr virus, polyomavirus, TT virus, etc. In a preferred embodiment, the method is applied to inhibit viral budding by hepatitis B virus, hepatitis E virus, and human herpes virus 1. By inhibiting viral budding in cells in a patient, the viral load in the patient body can be prevented from increasing and can even be decreased. Accordingly, the method of the present invention can also be used in treating viral infection as well as symptoms caused by and/or associated with the viral infection. In addition, when applied at an early stage before a patient develops a full-blown disease caused by viral infection, the method can be used to prevent such a disease by inhibiting viral propagation and decreasing the viral load in the patient. For example, human hepatitis B virus is known to cause hepatitis which may increase the risk of liver cancer. Thus, if the compounds of the present invention is applied to a patient at an early stage of the hepatitis B infection before the full-blown of hepatitis, hepatitis may be prevented and the likelihood of liver cancer in the patient may be reduced.

The compounds according to the present invention can be of any type of chemical compounds. For example, the compound can be a peptide, a modified peptide, an oligonucleotide-peptide hybrid (e.g., PNA), etc. In a preferred embodiment, the compound administered is capable of binding a type I WW-domain of human Nedd4 or a Nedd4-like protein. In a specific aspect of this embodiment, the compound is a peptide having a PPXY motif. Advantageously, X is selected from the group consisting of proline (P), alanine (A), glutamic acid (E), asparagine (N), and arginine (R).

Thus, the compounds can be a tetrapeptide, e.g., having an amino acid sequence of PX₁X₂X₃ For example, the compounds can have an amino acid sequence of PPPY (SEQ ID NOs:1), PPAY (SEQ ID NO:2), PPNY (SEQ ID NO:3), PPRY (SEQ ID NO:4), all of which are derived from the rENaC P2 peptide. See Kanelis et al., Nat. Struct. Biol., 8:407-412 (2001).

The compound can also include a longer peptide comprising the amino acid sequence motif of PX₁X₂X₃. For example, the compound may include a peptide of 5, 6, 7, 8 or 9 amino acids, preferably 10, 11, 12, 13, 14, 15, 16, 17, 18, 19, 20 or more amino acids. Advantageously, the compound is a peptide that contains an amino acid sequence of less than about 400, 375, 350, 325, 300, 275, 250, 225 or 200 residues. Preferably, the peptide contains an amino acid sequence of less than about 175, 150, 125, 115, 100, 95, 90, 85, 80, 75, 70, 65, 60 or 55 residues. More preferably, the peptide contains an amino acid sequence of less than about 50, 48, 45, 42, 40, 38, 35, 33, 32, 31, 30, 29, 28, 27, 26, 25, 24, 23, 22, 21 or 20 residues. In preferred embodiments, the peptide contains an amino acid sequence of from about 4 to about 200, 6 to about 150, 8 to about 100, preferably from about 8 to about 50, more preferably from about 9 to about 50, from about 9 to 45, 9 to 40, 9 to 37, 9 to 35, 9 to 30, 9 to 25 residues. More advantageously, the peptide contains an amino acid sequence of from 9 to about 10, 11, 12, 13, 14, 15, 16, 17, 18, 19, 20, 21, 22, 23 or 24 residues, even more advantageously, from 10 to about 11, 12, 13, 14, 15, 16, 17, 18, 19, 20, 21, 22, 23 or 24 residues. Preferably, the PX₁X₂X₃ motif in the sequence is the PPXY motif.

Preferred examples of pentapeptides include but are not limited to PPPAY (SEQ ID NO:5), PPPNY (SEQ ID NO:6), and PPPRY (SEQ ID NO:7).

In one embodiment, the compound includes a peptide that contains a contiguous amino acid sequence of a naturally occurring rENaC P2 peptide sequence. The contiguous span should span at least one of the PY motifs of the rENaC P2 peptide. In another embodiment, the compound includes a peptide that contains a contiguous amino acid sequence of a naturally occurring peptide sequence of Rous sarcoma virus p2b, which contiguous sequence should span the PY motif in the p2b protein. In yet another embodiment, the compound includes a peptide that contains a contiguous amino acid sequence of a naturally occurring peptide sequence of Moloney murine leukemia virus (M-MuLV) p12 protein, which contiguous sequence should span the PY motif in the p12 protein. In yet another embodiment, the compound includes a peptide that contains a contiguous amino acid sequence of a naturally occurring peptide sequence of Mason-Pfizer money virus (M-PMV) pp24/16, which contiguous sequence should span the PY motif in the pp24/16 protein. See Yasuda and Hunter, J. Virol., 72:4095-4103 (1998).

In specific embodiments, the compound includes an amino acid sequence selected from the group of PPPNYD (SEQ ID NO:8), PPPNYDS (SEQ ID NO:9), PPPNYDSL (SEQ ID NO: 10), TPPPNY (SEQ ID NO: 11), TPPPNYD (SEQ ID NO: 12), TPPPNYDS (SEQ ID NO: 13), TPPPNYDSL (SEQ ID NO: 14), GTPPPNY (SEQ ID NO:15), PGTPPPNY (SEQ ID NO:16), GTPPPNYDS (SEQ ID NO: 17), GTPPPNYDSL (SEQ ID NO:18), PGTPPPNYDSL (SEQ ID NO: 19), IPGTPPPNYDSL (SEQ ID NO:20), PIPGTPPPNYDSL (SEQ ID NO:21), LPIPGTPPPNYDSL (SEQ ID NO:22), TLPIPGTPPPNYDSL (SEQ ID NO:23), GTPPPNYD (SEQ ID NO:24), PPPAYATL (SEQ ID NO:25), and PPPRYNTL (SEQ ID NO:26).

In another embodiment, the compound includes a contiguous amino acid sequence of a viral protein selected from the group consisting of matrix proteins of rhabdoviruses, matrix proteins of filoviruses, Rous Sarcoma virus GAG protein, Mason-Pfizer Monkey virus GAG protein, hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, and TT virus ORF2 protein, and wherein the contiguous amino acid sequence encompasses the PPXY motif of the viral protein.

In a specific embodiment, the compound includes a contiguous amino acid sequence of VSV matrix protein, Rous Sarcoma virus GAG protein or Mason-Pfizer Monkey virus GAG protein that encompasses the PPXY motif of the protein.

Advantageously, the compound is a peptide that contains a contiguous amino acid sequence of less than about 400, 375, 350, 325, 300, 275, 250, 225 or 200 residues of one of the viral proteins in Table 1, which encompasses the PPXY motif of the viral protein, and is capable of binding a Type I WW-domain of Nedd4. Preferably, the peptide contains a contiguous amino acid sequence of less than about 175, 150, 125, 115, 100, 95, 90, 85, 80, 75, 70, 65, 60 or 55 residues of one of the viral proteins in Table 1, which encompasses the PPXY motif of the viral protein, and is capable of binding a Type I WW-domain of Nedd4. More preferably, the peptide contains a contiguous amino acid sequence of less than about 50, 48, 45, 42, 40, 38, 35, 33, 32, 31, 30, 29, 28, 27, 26, 25, 24, 23, 22, 21 or 20 residues of one of the viral proteins in Table 1, which encompasses the PPXY motif of the viral protein, and is capable of binding a Type I WW-domain of Nedd4. In preferred embodiments, the peptide contains a contiguous amino acid sequence of from about 4 to about 50, preferably from about 6 to about 50, from about 8 to about 50, more preferably from about 9 to about 50, from about 9 to 45, 9 to 40, 9 to 37, 9 to 35, 9 to 30, 9 to 25 residues of one of the viral proteins in Table 1, which encompasses the PPXY motif of the viral protein, and is capable of binding a Type I WW-domain of Nedd4. More advantageously, the peptide contains a contiguous amino acid sequence of from 9 to about 10, 11, 12, 13, 14, 15, 16, 17, 18, 19, 20, 21, 22, 23 or 24 residues of a viral protein in Table 1, even more advantageously, from 10 to about 11, 12, 13, 14, 15, 16, 17, 18, 19, 20, 21, 22, 23 or 24 residues of one of the viral proteins in Table 1, which encompasses the PPXY motif of the viral protein, and is capable of binding a Type I WW-domain of Nedd4.

In specific embodiments, a peptide according to the present invention has a contiguous amino acid sequence of a viral protein in Table I as provided in SEQ ID NOs:39-153, SEQ ID NOs:154-295, SEQ ID NOs:296-438, SEQ ID NOs:439-581, SEQ ID NOs:582-724, SEQ ID NOs:725-1010, SEQ ID NOs:1011-1296, SEQ ID NOs:1297-1439, SEQ ID NOs:1440-1452, SEQ ID NOs:1453-1491, SEQ ID NOs:1492-1530, and SEQ ID NOs:1531-1673.

In another embodiment, the compound according to the present invention is within an amino acid sequence that is at least 70 percent, preferably at least 80 percent or 85 percent, more preferably at least 90 percent or 95 percent identical to a contiguous span of at least 5, 6, 7, 8 or 9 amino acids, preferably 10, 11, 12, 13, 14, 15, 16, 17, 18, 19, 20 or more amino acids of one of the proteins in Table 1, which contiguous span of amino acids spans the late domain motif PPXY. In another embodiment, the compound according to the present invention is within an amino acid sequence that is at least 70 percent, preferably at least 80 percent or 85 percent, more preferably at least 90 percent or 95 percent identical to a contiguous span of at least 5, 6, 7, 8 or 9 amino acids, preferably 10, 11, 12, 13, 14, 15, 16, 17, 18, 19, 20 or more amino acids of a naturally occuring Moloney murine leukemia virus (M-MuLV) p12 protein, which contiguous span of amino acids spans the late domain motif PPPY of p12. In yet another embodiment, the compound according to the present invention is within an amino acid sequence that is at least 70 percent, preferably at least 80 percent or 85 percent, more preferably at least 90 percent or 95 percent identical to a contiguous span of at least 5, 6, 7, 8 or 9 amino acids, preferably 10, 11, 12, 13, 14, 15, 16, 17, 18, 19, 20 or more amino acids of a naturally occuring Mason-Pfizer money virus (M-PMV) pp24/16, which contiguous span of amino acids spans the late domain motif PPPY of pp24/16. In this respect, the percentage identity is determined by the algorithm of Karlin and Altschul, Proc. Natl. Acad. Sci. USA, 90:5873-77 (1993), which is incorporated into the various BLAST programs. Specifically, the percentage identity is determined by the “BLAST 2 Sequences” tool, which is available at http://www.ncbi.nlm.nih.gov/gorf/bl2.html. See Tatusova and Madden, FEMS Microbiol. Lett., 174(2):247-50 (1999). For pairwise protein-protein sequence comparison, the BLASTP 2.1.2 program is employed using default parameters (Matrix: BLOSUM62; gap open: 11; gap extension: 1; x_dropoff: 15; expect: 10.0; and wordsize: 3, with filter). Preferably, such homologue peptides retain the ability to bind a type I WW-domain of Nedd4 or a Nedd4-like protein. Preferably, in such embodiments of the present invention, X₁ in the PX₁X₂X₃ motif is P or an analog thereof. More preferably, X₁ is P or an analog thereof, and X₃ is Y or W or an analog thereof. Most preferably, X₁ is P or an analog thereof, X₂ is P or an analog thereof, and X₃ is Y or W or an analog thereof.

The homologues can be made by site-directed mutagenesis based on, e.g., a late domain motif-containing Rous sarcoma virus p2b peptide or another late domain-containing viral protein, or on a late domain motif-containing sequence of a protein in Table 1. The site-directed mutagenesis can be designed to generate amino acid substitutions, insertions, or deletions. Methods for conducting such mutagenesis should be apparent to skilled artisans in the field of molecular biology. The resultant homologues can be tested for their binding affinity to a type I WW-domain of Nedd4 or of a Nedd4-like protein.

The peptide portion in the compounds according to the present invention can also be in a modified form. Various modifications may be made to improve the stability and solubility of the compound, and/or optimize its binding affinity to a type I WW-domain of Nedd4. Examples of modified forms include, but are not limited to, glycosylated forms, phosphorylated forms, myristoylated forms, palmitoylated forms, ribosylated forms, acetylated forms, etc. Modifications also include intra-molecular crosslinking and covalent attachment to various moieties such as lipids, flavin, biotin, polyethylene glycol or derivatives thereof, etc. In addition, modifications may also include cyclization, and branching. Amino acids other than the conventional twenty amino acids encoded by genes may also be included in a polypeptide sequence in the compound of the present invention. For example, the compounds may include D-amino acids in place of L-amino acids.

To increase the stability of the compounds according to the present invention, various protection groups can also be incorporated into the amino acid residues of the compounds. In particular, terminal residues are preferably protected. Carboxyl groups may be protected by esters (e.g., methyl, ethyl, benzyl, p-nitrobenzyl, t-butyl or t-amyl esters, etc.), lower alkoxyl groups (e.g., methoxy, ethoxy, propoxy, butoxy, etc.), aralkyloxy groups (e.g., benzyloxy, etc.), amino groups, lower alkylamino or di(lower alkyl)amino groups. The term “lower alkoxy” is intended to mean an alkoxy group having a straight, branched or cyclic hydrocarbon moiety of up to six carbon atoms. Protection groups for amino groups may include lower alkyl, benzyloxycarbonyl, t-butoxycarbonyl, and sobornyloxycarbonyl. “Lower alkyl” is intended to mean an alkyl group having a straight, branched or cyclic hydrocarbon moiety of up to six carbon atoms. In one example, a 5-oxo-L-prolyl residue may be used in place of a prolyl residue. A 5-oxo-L-prolyl residue is especially desirable at the N-terminus of a peptide compound. In another example, when a proline residue is at the C-terminus of a peptide compound, a N-ethyl-L-prolinamide residue may be desirable in place of the proline residue. Various other protection groups known in the art useful in increasing the stability of peptide compounds can also be employed.

In addition, the compounds according to the present invention can also be in various pharmaceutically acceptable salt forms. “Pharmaceutically acceptable salts” refers to the relatively non-toxic, organic or inorganic salts of the compounds of the present invention, including inorganic or organic acid addition salts of the compound. Examples of such salts include, but are not limited to, hydrochloride salts, hydrobromide salts, sulfate salts, bisulfate salts, nitrate salts, acetate salts, phosphate salts, nitrate salts, oxalate salts, valerate salts, oleate salts, borate salts, benzoate salts, laurate saltes, stearate salts, palmitate salts, lactate salts, tosylate salts, citrate salts, maleate, salts, succinate salts, tartrate salts, naththylate salts, fumarate salts, mesylate salts, laurylsuphonate salts, glucoheptonate salts, and the like. See, e.g., Berge, et al. J. Pharm. Sci., 66:1-19 (1977).

Suitable pharmaceutically acceptable salts also include, but are not limited to, alkali metal salts, alkaline earth salts, and ammonium salts. Thus, suitable salts may be salts of aluminum, calcium, lithium, magnesium, potassium, sodium and zinc. In addition, organic salts may also be used including, e.g., salts of lysine, N,N′-dibenzylethylenediamine, chloroprocaine, choline, diethanolamine, ethylenediamine, meglumine (N-methylglucamine), procaine and tris. In addition, metal complex forms (e.g. copper complex compounds, zinc complex compounds, etc.) of the compounds of the present invention may also exhibit improved stability.

Additionally, as will be apparent to skilled artisans apprised of the present disclosure, peptide mimetics can be designed based on the above-described compounds according to the present invention. However, it is noted that the mimetics preferably are capable of binding a type I WW-domain of Nedd4 or a Nedd4-like protein. For example, peptoid analogs of the PPPY motif can be prepared using known methods. Peptoids are oligomeric N-substituted glycines. Typically, various side chain groups can be included when forming an N-substituted glycine (peptoid monomer) that mimics a particular amino acid. Peptoid monomers can be linked together to form an oligomeric N-substituted glycines-peptoid. Peptoids are easy to synthesize in large amounts. In contrast to peptides, the backbone linkage of peptoids are resistant to hydrolytic enzymes. In addition, since a variety of functional groups can be presented as side chains off of the oligomeric backbone, peptoid analogs corresponding to any peptides can be produced with improved characterics. See Simon et al., Proc. Natl. Acad. Sci. USA, 89:9367-9371 (1992); Figliozzi et al., Methods Enzymol., 267:437-447 (1996); Horwell, Trends Biotechnol., 13:132-134 (1995); and Horwell, Drug Des. Discov., 12:63-75 (1994), all of which are incorporated herein by reference.

Thus, peptoid analogs of the above-described compounds of the present invention can be made using methods known in the art. The thus prepared peptoid analogs can be tested for their binding affinity to a type I WW-domain of Nedd4. They can also be tested in antiviral assays for their ability to inhibit viral budding from infected host cells and ability to inhibit viral propagation.

Mimetics of the compounds of the present invention can also be selected by rational drug design and/or virtual screening. Methods known in the art for rational drug design can be used in the present invention. See, e.g., Hodgson et al., Bio/Technology, 9:19-21 (1991); U.S. Pat. Nos. 5,800,998 and 5,891,628, all of which are incorporated herein by reference. An example of rational drug design is the development of HIV protease inhibitors. See Erickson et al., Science, 249:527-533 (1990). Structural information on a type I WW-domain of Nedd4 in complex with a PY motif-containing EnaC peptide is disclosed in Kanelis et al., Nat. Struct. Biol., 8:407-412 (2001), which is incorporated herein by reference. Structural information on the binding complex formed by the Nedd4 WW domain and the PPPY motif in a protein in Table 1 can also be obtained. The interacting complex can be studied using various biophysics techniques including, e.g., X-ray crystallography, NMR, computer modeling, mass spectrometry, and the like. Likewise, structural information can also be obtained from protein complexes formed by the Nedd4 WW domain and a variation of the PPPY motif.

Computer programs are employed to select compounds based on structural models. In addition, once an effective compound is identified, structural analogs or mimetics thereof can be produced based on rational drug design with the aim of improving drug efficacy and stability, and reducing side effects.

In addition, understanding of the interaction between a type I WW-domain of Nedd4 and compounds of the present invention can also be derived from mutagenesis analysis using yeast two-hybrid system or other methods for detection protein-protein interaction. In this respect, various mutations can be introduced into the interacting proteins and the effect of the mutations on protein-protein interaction is examined by a suitable method such as in vitro binding assay or the yeast two-hybrid system.

Various mutations including amino acid substitutions, deletions and insertions can be introduced into the protein sequence of a type I Nedd4 WW domain and/or a compound of the present invention using conventional recombinant DNA technologies. Generally, it is particularly desirable to decipher the protein binding sites. Thus, it is important that the mutations introduced only affect protein-protein interaction and cause minimal structural disturbances. Mutations are preferably designed based on knowledge of the three-dimensional structure of the interacting proteins. Preferably, mutations are introduced to alter charged amino acids or hydrophobic amino acids exposed on the surface of the proteins, since ionic interactions and hydrophobic interactions are often involved in protein-protein interactions. Alternatively, the “alanine scanning mutagenesis” technique is used. See Wells, et al., Methods Enzymol., 202:301-306 (1991); Bass et al., Proc. Natl. Acad. Sci. USA, 88:4498-4502 (1991); Bennet et al., J. Biol. Chem., 266:5191-5201 (1991); Diamond et al., J. Virol., 68:863-876 (1994). Using this technique, charged or hydrophobic amino acid residues of the interacting proteins are replaced by alanine, and the effect on the interaction between the proteins is analyzed using e.g., an in vitro binding assay. In this manner, the domains or residues of the proteins important to compound-target interaction can be identified.

Based on the structural information obtained, structural relationships between a type I Nedd4 WW domain and a compound of the present invention are elucidated. The moieties and the three-dimensional structures critical to the interaction are revealed. Medicinal chemists can then design analog compounds having similar moieties and structures.

The residues or domains critical to the modulating effect of the identified compound constitute the active region of the compound known as its “pharmacophore.”Once the pharmacophore has been elucidated, a structural model can be established by a modeling process that may incorporate data from NMR analysis, X-ray diffraction data, alanine scanning, spectroscopic techniques and the like. Various techniques including computational analysis, similarity mapping and the like can all be used in this modeling process. See e.g., Perry et al., in OSAR: Quantitative Structure-Activity Relationships in Drug Design, pp. 189-193, Alan R. Liss, Inc., 1989; Rotivinen et al., Acta Pharmaceutical Fennica, 97:159-166 (1988); Lewis et al., Proc. R. Soc. Lond., 236:125-140 (1989); McKinaly et al., Annu. Rev. Pharmacol. Toxiciol., 29:111-122 (1989). Commercial molecular modeling systems available from Polygen Corporation, Waltham, Mass., include the CHARMm program, which performs the energy minimization and molecular dynamics functions, and QUANTA program which performs the construction, graphic modeling and analysis of molecular structure. Such programs allow interactive construction, visualization and modification of molecules. Other computer modeling programs are also available from BioDesign, Inc. (Pasadena, Calif.), Hypercube, Inc. (Cambridge, Ontario), and Allelix, Inc. (Mississauga, Ontario, Canada).

A template can be formed based on the established model. Various compounds can then be designed by linking various chemical groups or moieties to the template. Various moieties of the template can also be replaced. These rationally designed compounds are further tested. In this manner, pharmacologically acceptable and stable compounds with improved efficacy and reduced side effect can be developed. The compounds identified in accordance with the present invention can be incorporated into a pharmaceutical formulation suitable for administration to an individual.

The mimetics including peptoid analogs can exhibit optimal binding affinity to a type I WW domain of human Nedd4 or animal orthologs thereof. Various known methods can be utilized to test the Nedd4-binding characteristics of a mimetics. For example, the entire Nedd4 protein or a fragment thereof containing a type I WW domain may be recombinantly expressed, purified, and contacted with the mimetics to be tested. Binding can be determined using a surface plasmon resonance biosensor. See e.g., Panayotou et al., Mol. Cell. Biol., 13:3567-3576 (1993). Other methods known in the art for estimating and determining binding constants in protein-protein interactions can also be employed. See Phizicky and Fields, et al., Microbiol. Rev., 59:94-123 (1995). For example, protein affinity chromatography may be used. First, columns are prepared with different concentrations of an interacting member, which is covalently bound to the columns. Then a preparation of its interacting partner is run through the column and washed with buffer. The interacting partner bound to the interacting member linked to the column is then eluted. Binding constant is then estimated based on the concentrations of the bound protein and the eluted protein. Alternatively, the method of sedimentation through gradients monitors the rate of sedimentation of a mixture of proteins through gradients of glycerol or sucrose. At concentrations above the binding constant, the two interacting members sediment as a complex. Thus, binding constant can be calculated based on the concentrations. Other suitable methods known in the art for estimating binding constant include but are not limited to gel filtration column such as nonequilibrium “small-zone” gel filtration columns (See e.g., Gill et al., J. Mol. Biol., 220:307-324 (1991)), the Hummel-Dreyer method of equilibrium gel filtration (See e.g., Hummel and Dreyer, Biochim. Biophys. Acta, 63:530-532 (1962)) and large-zone equilibrium gel filtration (See e.g., Gilbert and Kellett, J. Biol. Chem., 246:6079-6086 (1971)), sedimentation equilibrium (See e.g., Rivas and Minton, Trends Biochem., 18:284-287 (1993)), fluorescence methods such as fluorescence spectrum (See e.g., Otto-Bruc et al, Biochemistry, 32:8632-8645 (1993)) and fluorescence polarization or anisotropy with tagged molecules (See e.g., Weiel and Hershey, Biochemistry, 20:5859-5865 (1981)), and solution equilibrium measured with immobilized binding protein (See e.g., Nelson and Long, Biochemistry, 30:2384-2390 (1991)).

The compounds according the present invention can be delivered into cells by direct cell internalization, receptor mediated endocytosis, or via a “transporter.” It is noted that the compound administered to cells in vitro or in vivo in the method of the present invention preferably is delivered into the cells in order to achieve optimal results. Thus, preferably, the compound to be delivered is associated with a transporter capable of increasing the uptake of the compound by a mammalian cell, preferably a human cell, susceptible to infection by a virus, particularly a virus selected from those in Table 1. As used herein, the term “associated with” means a compound to be delivered is physically associated with a transporter. The compound and the transporter can be covalently linked together, or associated with each other as a result of physical affinities such as forces caused by electrical charge differences, hydrophobicity, hydrogen bonds, van der Waals force, ionic force, or a combination thereof. For example, the compound can be encapsulated within a transporter such as a cationic liposome.

As used herein, the term “transporter” refers to an entity (e.g., a compound or a composition or a physical structure formed from multiple copies of a compound or multiple different compounds) that is capable of facilitating the uptake of a compound of the present invention by a mammalian cell, particularly a human cell. Typically, the cell uptake of a compound of the present invention in the presence of a “transporter” is at least 50% higher than the cell uptake of the compound in the absence of the “transporter.” Preferably, the cell uptake of a compound of the present invention in the presence of a “transporter” is at least 75% higher, preferably at least 100% or 200% higher, and more preferably at least 300%, 400% or 500% higher than the cell uptake of the compound in the absence of the “transporter.” Methods of assaying cell uptake of a compound should be apparent to skilled artisans. For example, the compound to be delivered can be labeled with a radioactive isotope or another detectable marker (e.g., a fluorescence marker), and added to cultured cells in the presence or absence of a transporter, and incubated for a time period sufficient to allow maximal uptake. Cells can then be separated from the culture medium and the detectable signal (e.g., radioactivity) caused by the compound inside the cells can be measured. The result obtained in the presence of a transporter can be compared to that obtained in the absence of a transporter.

Many molecules and structures known in the art can be used as “transporter.” In one embodiment, a penetratin is used as a transporter. For example, the homeodomain of Antennapedia, a Drosophila transcription factor, can be used as a transporter to deliver a compound of the present invention. Indeed, any suitable member of the penetratin class of peptides can be used to carry a compound of the present invention into cells. Penetratins are disclosed in, e.g., Derossi et al., Trends Cell Biol., 8:84-87 (1998), which is incorporated herein by reference. Penetratins transport molecules attached thereto across cytoplasm membranes or nucleus membranes efficiently in a receptor-independent, energy-independent, and cell type-independent manner. Methods for using a penetratin as a carrier to deliver oligonucleotides and polypeptides are also disclosed in U.S. Pat. No. 6,080,724; Pooga et al., Nat. Biotech., 16:857 (1998); and Schutze et al., J. Immunol., 157:650 (1996), all of which are incorporated herein by reference. U.S. Pat. No. 6,080,724 defines the minimal requirements for a penetratin peptide as a peptide of 16 amino acids with 6 to 10 of which being hydrophobic. The amino acid at position 6 counting from either the N- or C-terminal is tryptophan, while the amino acids at positions 3 and 5 counting from either the N- or C-terminal are not both valine. Preferably, the helix 3 of the homeodomain of Drosophila Antennapedia is used as a transporter. More preferably, a peptide having a sequence of the amino acids 43-58 of the homeodomain Antp is employed as a transporter. In addition, other naturally occurring homologs of the helix 3 of the homeodomain of Drosophila Antennapedia can also be used. For example, homeodomains of Fushi-tarazu and Engrailed have been shown to be capable of transporting peptides into cells. See Han et al., Mol. Cells, 10:728-32 (2000). As used herein, the term “penetratin” also encompasses peptoid analogs of the penetratin peptides. Typically, the penetratin peptides and peptoid analogs thereof are covalently linked to a compound to be delivered into cells thus increasing the cellular uptake of the compound.

In another embodiment, the HIV-1 tat protein or a derivative thereof is used as a “transporter” covalently linked to a compound according to the present invention. The use of HIV-1 tat protein and derivatives thereof to deliver macromolecules into cells has been known in the art. See Green and Loewenstein, Cell, 55:1179 (1988); Frankel and Pabo, Cell, 55:1189 (1988); Vives et al., J. Biol. Chem., 272:16010-16017 (1997); Schwarze et al., Science, 285:1569-1572 (1999). It is known that the sequence responsible for cellular uptake consists of the highly basic region, amino acid residues 49-57. See e.g., Vives et al., J. Biol. Chem., 272:16010-16017 (1997); Wender et al., Proc. Nat'l Acad. Sci. USA, 97:13003-13008 (2000). The basic domain is believed to target the lipid bilayer component of cell membranes. It causes a covalently linked protein or nucleic acid to cross cell membrane rapidly in a cell type-independent manner. Proteins ranging in size from 15 to 120 kD have been delivered with this technology into a variety of cell types both in vitro and in vivo. See Schwarze et al., Science, 285:1569-1572 (1999). Any HIV tat-derived peptides or peptoid analogs thereof capable of transporting macromolecules such as peptides can be used for purposes of the present invention. For example, any native tat peptides having the highly basic region, amino acid residues 49-57 can be used as a transporter by covalently linking it to the compound to be delivered. In addition, various analogs of the tat peptide of amino acid residues 49-57 can also be useful transporters for purposes of this invention. Examples of various such analogs are disclosed in Wender et al., Proc. Nat'l. Acad. Sci. USA, 97:13003-13008 (2000) (which is incorporated herein by reference) including, e.g., d-Tat₄₉₋₅₇, retro-inverso isomers of l- or d-Tat₄₉₋₅₇ (i.e., l-Tat₅₇₋₄₉ and d-Tat₅₇₋₄₉), L-arginine oligomers, D-arginine oligomers, L-lysine oligomers, D-lysine oligomers, L-histine oligomers, D-histine oligomers, L-ornithine oligomers, D-ornithine oligomers, and various homologues, derivatives (e.g., modified forms with conjugates linked to the small peptides) and peptoid analogs thereof. Typically, arginine oligomers are preferred to the other oligomers, arginine oligomers are much more efficient in promoting cellular uptake. As used herein, the term “oligomer” means a molecule that includes a covalently linked chain of amino acid residues of the same amino acids having a large enough number of such amino acid residues to confer transporter activities on the molecule. Typically, an oligomer contains at least 6, preferably at least 7, 8, or at least 9 such amino acid residues. In one embodiment, the transporter is a peptide that includes at least six contiguous amino acid residues that are a combination of two or more of L-arginine, D-arginine, L-lysine, D-lysine, L-histidine, D-histine, L-ornithine, and D-ornithine.

Other useful transporters known in the art include, but are not limited to, short peptide sequences derived from fibroblast growth factor (See Lin et al., J. Biol. Chem., 270:14255-14258 (1998)), Galparan (See Pooga et al., FASEB J. 12:67-77 (1998)), and HSV-1 structural protein VP22 (See Elliott and O'Hare, Cell, 88:223-233 (1997)).

In addition to peptide-based transporters, various other types of transporters can also be used, including but not limited to cationic liposomes (see Rui et al., J. Am. Chem. Soc., 120:11213-11218 (1998)), dendrimers (Kono et al., Bioconjugate Chem., 10:1115-1121 (1999)), siderophores (Ghosh et al., Chem. Biol., 3:1011-1019 (1996)), etc. In a specific embodiment, the compound according to the present invention is encapsulated into liposomes for delivery into cells.

Additionally, when a compound according to the present invention is a peptide, it can be introduced into cells by a gene therapy method. That is, a nucleic acid encoding the peptide can be administered to in vitro cells or to cells in vivo in a human or animal body. The nucleic acid encoding the peptide may or may not also encode a peptidic transporter as described above. Various gene therapy methods are well known in the art. Successes in gene therapy have been reported recently. See e.g., Kay et al., Nature Genet., 24:257-61 (2000); Cavazzana-Calvo et al., Science, 288:669 (2000); and Blaese et al., Science, 270: 475 (1995); Kantoff, et al., J. Exp. Med., 166:219 (1987).

In one embodiment, the peptide consists of a contiguous amino acid sequence of from 8 to about 30 amino acid residues of a viral protein selected from the group consisting of hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, and TT virus ORF2 protein, wherein the contiguous amino acid sequence encompasses the PPXY motif of the viral protein, and wherein the peptide is capable of binding a type I WW-domain of the Nedd4 protein. Preferably, the peptide consists of at least 9, 10, 11, 12, 13, 14, or 15 amino acids. Also preferably, the peptide consists of no greater than 25, 24, 23, 22, 21, 20, 19, 18, 17, 16 or 15 amino acids. More preferably, the peptide consists of from 9 to 20, 23 or 25 amino acids, or from 10 or 11 to 20, 23 or 25 amino acids.

For example, the peptide can include an amino acid sequence selected from the group consisting of SEQ ID NOs:24-36, SEQ ID NOs:154-295, SEQ ID NOs:296-438, SEQ ID NOs:439-581, SEQ ID NOs:582-724, SEQ ID NOs:725-1010, SEQ ID NOs:1011-1296, SEQ ID NOs:1297-1439, SEQ ID NOs:1440-1452, SEQ ID NOs:1453-1491, SEQ ID NOs:1492-1530, and SEQ ID NOs:1531-1673.

Any suitable gene therapy methods may be used for purposes of the present invention. Generally, an exogenous nucleic acid encoding a peptide compound of the present invention is incorporated into a suitable expression vector and is operably linked to a promoter in the vector. Suitable promoters include but are not limited to viral transcription promoters derived from adenovirus, simian virus 40 (SV40) (e.g., the early and late promoters of SV40), Rous sarcoma virus (RSV), and cytomegalovirus (CMV) (e.g., CMV immediate-early promoter), human immunodeficiency virus (HIV) (e.g., long terminal repeat (LTR)), vaccinia virus (e.g., 7.5K promoter), and herpes simplex virus (HSV) (e.g., thymidine kinase promoter). Where tissue-specific expression of the exogenous gene is desirable, tissue-specific promoters may be operably linked to the exogenous gene. In addition, selection markers may also be included in the vector for purposes of selecting, in vitro, those cells that contain the exogenous nucleic acid encoding the peptide compound of the present invention. Various selection markers known in the art may be used including, but not limited to, e.g., genes conferring resistance to neomycin, hygromycin, zeocin, and the like.

In one embodiment, the exogenous nucleic acid is incorporated into a plasmid DNA vector. Many commercially available expression vectors may be useful for the present invention, including, e.g., pCEP4, pcDNAI, pIND, pSecTag2, pVAX1, pcDNA3.1, and pBI-EGFP, and pDisplay.

Various viral vectors may also be used. Typically, in a viral vector, the viral genome is engineered to eliminate the disease-causing capability, e.g., the ability to replicate in the host cells. The exogenous nucleic acid to be introduced into a patient may be incorporated into the engineered viral genome, e.g., by inserting it into a viral gene that is non-essential to the viral infectivity. Viral vectors are convenient to use as they can be easily introduced into tissue cells by way of infection. Once in the host cell, the recombinant virus typically is integrated into the genome of the host cell. In rare instances, the recombinant virus may also replicate and remain as extrachromosomal elements.

A large number of retroviral vectors have been developed for gene therapy. These include vectors derived from oncoretroviruses (e.g., MLV), viruses (e.g., HIV and SIV) and other retroviruses. For example, gene therapy vectors have been developed based on murine leukemia virus (See, Cepko, et al., Cell, 37:1053-1062 (1984), Cone and Mulligan, Proc. Natl. Acad. Sci. U.S.A., 81:6349-6353 (1984)), mouse mammary tumor virus (See, Salmons et al., Biochem. Biophys. Res. Commun., 159:1191-1198 (1984)), gibbon ape leukemia virus (See, Miller et al., J. Virology, 65:2220-2224 (1991)), HIV, (See Shimada et al., J. Clin. Invest., 88:1043-1047 (1991)), and avian retroviruses (See Cosset et al., J. Virology, 64:1070-1078 (1990)). In addition, various retroviral vectors are also described in U.S. Pat. Nos. 6,168,916; 6,140,111; 6,096,534; 5,985,655; 5,911,983; 4,980,286; and 4,868,116, all of which are incorporated herein by reference.

Adeno-associated virus (AAV) vectors have been successfully tested in clinical trials. See e.g., Kay et al., Nature Genet. 24:257-61 (2000). AAV is a naturally occurring defective virus that requires other viruses such as adenoviruses or herpes viruses as helper viruses. See Muzyczka, Curr. Top. Microbiol. Immun., 158:97 (1992). A recombinant AAV virus useful as a gene therapy vector is disclosed in U.S. Pat. No. 6,153,436, which is incorporated herein by reference.

Adenoviral vectors can also be useful for purposes of gene therapy in accordance with the present invention. For example, U.S. Pat. No. 6,001,816 discloses an adenoviral vector, which is used to deliver a leptin gene intravenously to a mammal to treat obesity. Other recombinant adenoviral vectors may also be used, which include those disclosed in U.S. Pat. Nos. 6,171,855; 6,140,087; 6,063,622; 6,033,908; and 5,932,210, and Rosenfeld et al., Science, 252:431-434 (1991); and Rosenfeld et al., Cell, 68:143-155 (1992).

Other useful viral vectors include recombinant hepatitis viral vectors (See, e.g., U.S. Pat. No. 5,981,274), and recombinant entomopox vectors (See, e.g., U.S. Pat. Nos. 5,721,352 and 5,753,258).

Other non-traditional vectors may also be used for purposes of this invention. For example, International Publication No. WO 94/18834 discloses a method of delivering DNA into mammalian cells by conjugating the DNA to be delivered with a polyelectrolyte to form a complex. The complex may be microinjected into or taken up by cells.

The exogenous nucleic acid fragment or plasmid DNA vector containing the exogenous gene may also be introduced into cells by way of receptor-mediated endocytosis. See e.g., U.S. Pat. No. 6,090,619; Wu and Wu, J. Biol. Chem., 263:14621 (1988); Curiel et al., Proc. Natl. Acad. Sci. USA, 88:8850 (1991). For example, U.S. Pat. No. 6,083,741 discloses introducing an exogenous nucleic acid into mammalian cells by associating the nucleic acid to a polycation moiety (e.g., poly-L-lysine, having 3-100 lysine residues), which is itself coupled to an integrin receptor binding moiety (e.g., a cyclic peptide having the amino acid sequence RGD).

Alternatively, the exogenous nucleic acid or vectors containing it can also be delivered into cells via amphiphiles. See e.g., U.S. Pat. No. 6,071,890. Typically, the exogenous nucleic acid or a vector containing the nucleic acid forms a complex with the cationic amphiphile. Mammalian cells contacted with the complex can readily absorb the complex.

The exogenous nucleic acid can be introduced into a patient for purposes of gene therapy by various methods known in the art. For example, the exogenous nucleic acid alone or in a conjugated or complex form described above, or incorporated into viral or DNA vectors, may be administered directly by injection into an appropriate tissue or organ of a patient. Alternatively, catheters or like devices may be used for delivery into a target organ or tissue. Suitable catheters are disclosed in, e.g., U.S. Pat. Nos. 4,186,745; 5,397,307; 5,547,472; 5,674,192; and 6,129,705, all of which are incorporated herein by reference.

In addition, the exogenous nucleic acid encoding a peptide compound of the present invention or vectors containing the nucleic acid can be introduced into isolated cells using any known techniques such as calcium phosphate precipitation, microinjection, lipofection, electroporation, gene gun, receptor-mediated endocytosis, and the like. Cells expressing the exogenous gene may be selected and redelivered back to the patient by, e.g., injection or cell transplantation. The appropriate amount of cells delivered to a patient will vary with patient conditions, and desired effect, which can be determined by a skilled artisan. See e.g., U.S. Pat. Nos. 6,054,288; 6,048,524; and 6,048,729. Preferably, the cells used are autologous, i.e., obtained from the patient being treated.

When the transporter used in the method of the present invention is a peptidic transporter, a hybrid polypeptide or fusion polypeptide is provided. In preferred embodiments, the hybrid polypeptide includes (a) a first portion comprising an amino acid sequence motif PPXY, and capable of binding a type I WW-domain of Nedd4, wherein X is an amino acid, preferably is proline, alanine, glutamic acid, asparagine or arginine, and (b) a second portion which is a peptidic transporter capable of increasing the uptake of the first portion by a human cell.

In one embodiment, the hybrid polypeptide includes from about 8 to about 100 amino acid residues, preferably 9 to 50 amino acid residues, more preferably 12 to 30 amino acid residues, and even more preferably from about 14 to 20 amino acid residues.

In a specific embodiment, the hybrid polypeptide does not contain a terminal L-histidine oligomer. As used herein, the term “terminal L-histidine oligomer” means an L-histidine oligomer at either of the two termini of the hybrid polypeptide, or at no more than one, two or three amino acid residues from either terminus of the hybrid polypeptide.

Preferably, the peptidic transporter is capable of increasing the uptake of the first portion by a mammalian cell by at least 100%, more preferably by at least 300%, 400% or 500%. In one embodiment, the first portion does not contain a contiguous amino acid sequence of a matrix protein of Ebola virus that is sufficient to impart an ability to bind the UEV domain of Tsg101 on the portion.

The hybrid polypeptide can be produced in a patient's body by administering to the patient a nucleic acid encoding the hybrid polypeptide by a gene therapy method as described above. Alternatively, the hybrid polypeptide can be chemically synthesized or produced by recombinant expression.

Thus, the present invention also provides isolated nucleic acids encoding the hybrid polypeptides and host cells containing the nucleic acid and recombinantly expressing the hybrid polypeptides. Such a host cell can be prepared by introducing into a suitable cell an exogenous nucleic acid encoding one of the hybrid polypeptides by standard molecular cloning techniques as described above. The nucleic acids can be prepared by linking a nucleic acid encoding the first portion and a nucleic acid encoding the second portion. Methods for preparing such nucleic acids and for using them in recombinant expression should be apparent to skilled artisans.

The compounds according to the present invention are a novel class of anti-viral compounds distinct from other commercially available compounds. While not wishing to be bound by any theory or hypothesis, it is believed that the compounds according to the present invention inhibit virus through a mechanism distinct from those of the anti-viral compounds known in the art. Therefore, it may be desirable to employ combination therapies to administer to a patient both a compound according to the present invention, with or without a transporter, and another anti-viral compound of a different class. However, it is to be understood that such other anti-viral compounds should be pharmaceutically compatible with the compound of the present invention. By “pharmaceutically compatible” it is intended that the other anti-viral agent(s) will not interact or react with the above composition, directly or indirectly, in such a way as to adversely affect the effect of the treatment, or to cause any significant adverse side reaction in the patient. In this combination therapy approach, the two different pharmaceutically active compounds can be administered separately or in the same pharmaceutical composition. Compounds suitable for use in combination therapies with the compounds according to the present invention include, but are not limited to, small molecule drugs, antibodies, immunomodulators, and vaccines.

Typically, a compound of the present invention is administered to a patient in a pharmaceutical composition, which typically includes one or more pharmaceutically acceptable carriers that are inherently nontoxic and non-therapeutic. That is, the compounds are used in the manufacture of medicaments for use in the methods of treating viral infection provided in the present invention.

The pharmaceutical composition according to the present invention may be administered to a subject needing treatment or prevention through any appropriate routes such as parenteral, oral, or topical administration. The active compounds of this invention are administered at a therapeutically effective amount to achieve the desired therapeutic effect without causing any serious adverse effects in the patient treated. Generally, the toxicity profile and therapeutic efficacy of therapeutic agents can be determined by standard pharmaceutical procedures in suitable cell models or animal models or human clinical trials. As is known in the art, the LD₅₀ represents the dose lethal to about 50% of a tested population. The ED₅₀ is a parameter indicating the dose therapeutically effective in about 50% of a tested population. Both LD₅₀ and ED₅₀ can be determined in cell models and animal models. In addition, the IC₅₀ may also be obtained in cell models and animal models, which stands for the circulating plasma concentration that is effective in achieving about 50% of the maximal inhibition of the symptoms of a disease or disorder. Such data may be used in designing a dosage range for clinical trials in humans. Typically, as will be apparent to skilled artisans, the dosage range for human use should be designed such that the range centers around the ED₅₀ and/or IC₅₀, but significantly below the LD₅₀ obtained from cell or animal models.

Typically, the compounds of the present invention can be effective at an amount of from about 0.01 microgram to about 5000 mg per day, preferably from about 1 microgram to about 2500 mg per day. However, the amount can vary with the body weight of the patient treated and the state of disease conditions. The active ingredient may be administered at once, or may be divided into a number of smaller doses to be administered at predetermined intervals of time. The suitable dosage unit for each administration of the compounds of the present invention can be, e.g., from about 0.01 microgram to about 2000 mg, preferably from about 1 microgram to about 1000 mg.

In the case of combination therapy, a therapeutically effective amount of another anti-viral compound can be administered in a separate pharmaceutical composition, or alternatively included in the pharmaceutical composition that contains a compound according to the present invention. The pharmacology and toxicology of many of such other anti-viral compounds are known in the art. See e.g., Physicians Desk Reference, Medical Economics, Montvale, N.J.; and The Merck Index, Merck & Co., Rahway, N.J. The therapeutically effective amounts and suitable unit dosage ranges of such compounds used in art can be equally applicable in the present invention.

It should be understood that the dosage ranges set forth above are exemplary only and are not intended to limit the scope of this invention. The therapeutically effective amount for each active compound can vary with factors including but not limited to the activity of the compound used, stability of the active compound in the patient's body, the severity of the conditions to be alleviated, the total weight of the patient treated, the route of administration, the ease of absorption, distribution, and excretion of the active compound by the body, the age and sensitivity of the patient to be treated, and the like, as will be apparent to a skilled artisan. The amount of administration can also be adjusted as the various factors change over time.

The active compounds according to this invention can be administered to patients to be treated through any suitable routes of administration. Advantageously, the active compounds are delivered to the patient parenterally, i.e., by intravenous, intramuscular, intraperiotoneal, intracisternal, subcutaneous, or intraarticular injection or infusion.

For parenteral administration, the active compounds can be formulated into solutions or suspensions, or in lyophilized forms for conversion into solutions or suspensions before use. Lyophilized compositions may include pharmaceutically acceptable carriers such as gelatin, DL-lactic and glycolic acids copolymer, D-mannitol, etc. To convert the lyophilized forms into solutions or suspensions, diluent containing, e.g., carboxymethylcellulose sodium, D-mannitol, polysorbate 80, and water may be employed. Lyophilized forms may be stored in, e.g., a dual chamber syringe with one chamber containing the lyophilized composition and the other chamber containing the diluent. In addition, the active ingredient(s) can also be incorporated into sterile lyophilized microspheres for sustained release. Methods for making such microspheres are generally known in the art. See U.S. Pat. Nos. 4,652,441; 4,728,721; 4,849,228; 4,917,893; 4,954,298; 5,330,767; 5,476,663; 5,480,656; 5,575,987; 5,631,020; 5,631,021; 5,643,607; and 5,716,640.

In a solution or suspension form suitable for parenteral administration, the pharmaceutical composition can include, in addition to a therapeutically or prophylactically effective amount of a compound of the present invention, a buffering agent, an isotonicity adjusting agent, a preservative, and/or an anti-absorbent. Examples of suitable buffering agent include, but are not limited to, citrate, phosphate, tartrate, succinate, adipate, maleate, lactate and acetate buffers, sodium bicarbonate, and sodium carbonate, or a mixture thereof. Preferably, the buffering agent adjusts the pH of the solution to within the range of 5-8. Examples of suitable isotonicity adjusting agents include sodium chloride, glycerol, mannitol, and sorbitol, or a mixture thereof. A preservative (e.g., anti-microbial agent) may be desirable as it can inhibit microbial contamination or growth in the liquid forms of the pharmaceutical composition. Useful preservatives may include benzyl alcohol, a paraben and phenol or a mixture thereof. Materials such as human serum albumin, gelatin or a mixture thereof may be used as anti-absorbents. In addition, conventional solvents, surfactants, stabilizers, pH balancing buffers, and antioxidants can all be used in the parenteral formulations, including but not limited to dextrose, fixed oils, glycerine, polyethylene glycol, propylene glycol, ascorbic acid, sodium bisulfite, and the like. The parenteral formulation can be stored in any conventional containers such as vials, ampoules, and syringes.

The active compounds can also be delivered orally in enclosed gelatin capsules or compressed tablets. Capsules and tablets can be prepared in any conventional techniques. For example, the active compounds can be incorporated into a formulation which includes pharmaceutically acceptable carriers such as excipients (e.g., starch, lactose), binders (e.g., gelatin, cellulose, gum tragacanth), disintegrating agents (e.g., alginate, Primogel, and corn starch), lubricants (e.g., magnesium stearate, silicon dioxide), and sweetening or flavoring agents (e.g., glucose, sucrose, saccharin, methyl salicylate, and peppermint). Various coatings can also be prepared for the capsules and tablets to modify the flavors, tastes, colors, and shapes of the capsules and tablets. In addition, liquid carriers such as fatty oil can also be included in capsules.

Other forms of oral formulations such as chewing gum, suspension, syrup, wafer, elixir, and the like can also be prepared containing the active compounds used in this invention. Various modifying agents for flavors, tastes, colors, and shapes of the special forms can also be included. In addition, for convenient administration by enteral feeding tube in patients unable to swallow, the active compounds can be dissolved in an acceptable lipophilic vegetable oil vehicle such as olive oil, corn oil and safflower oil.

The active compounds can also be administered topically through rectal, vaginal, nasal, bucal, or mucosal applications. Topical formulations are generally known in the art including creams, gels, ointments, lotions, powders, pastes, suspensions, sprays, drops and aerosols. Typically, topical formulations include one or more thickening agents, humectants, and/or emollients including but not limited to xanthan gum, petrolatum, beeswax, or polyethylene glycol, sorbitol, mineral oil, lanolin, squalene, and the like.

A special form of topical administration is delivery by a transdermal patch. Methods for preparing transdermal patches are disclosed, e.g., in Brown, et al., Annual Review of Medicine, 39:221-229 (1988), which is incorporated herein by reference.

The active compounds can also be delivered by subcutaneous implantation for sustained release. This may be accomplished by using aseptic techniques to surgically implant the active compounds in any suitable formulation into the subcutaneous space of the anterior abdominal wall. See, e.g., Wilson et al., J. Clin. Psych. 45:242-247 (1984). Sustained release can be achieved by incorporating the active ingredients into a special carrier such as a hydrogel. Typically, a hydrogel is a network of high molecular weight biocompatible polymers, which can swell in water to form a gel like material. Hydrogels are generally known in the art. For example, hydrogels made of polyethylene glycols, or collagen, or poly(glycolic-co-L-lactic acid) are suitable for this invention. See, e.g., Phillips et al., J. Pharmaceut. Sci., 73:1718-1720 (1984).

The active compounds can also be conjugated, i.e., covalently linked, to a water soluble non-immunogenic high molecular weight polymer to form a polymer conjugate. Preferably, such polymers do not undesirably interfere with the cellular uptake of the active compounds. Advantageously, such polymers, e.g., polyethylene glycol, can impart solubility, stability, and reduced immunogenicity to the active compounds. As a result, the active compound in the conjugate when administered to a patient, can have a longer half-life in the body, and exhibit better efficacy. In one embodiment, the polymer is a peptide such as albumin or antibody fragment Fc. PEGylated proteins are currently being used in protein replacement therapies and for other therapeutic uses. For example, PEGylated adenosine deaminase (ADAGEN®) is being used to treat severe combined immunodeficiency disease (SCIDS). PEGylated L-asparaginase (ONCAPSPAR®) is being used to treat acute lymphoblastic leukemia (ALL). A general review of PEG-protein conjugates with clinical efficacy can be found in, e.g., Burnham, Am. J. Hosp. Pharm., 15:210-218 (1994). Preferably, the covalent linkage between the polymer and the active compound is hydrolytically degradable and is susceptible to hydrolysis under physiological conditions. Such conjugates are known as “prodrugs” and the polymer in the conjugate can be readily cleaved off inside the body, releasing the free active compounds.

Alternatively, other forms controlled release or protection including microcapsules and nanocapsules generally known in the art, and hydrogels described above can all be utilized in oral, parenteral, topical, and subcutaneous administration of the active compounds.

Another preferable delivery form is using liposomes as carrier. Liposomes are micelles formed from various lipids such as cholesterol, phospholipids, fatty acids, and derivatives thereof. Active compounds can be enclosed within such micelles. Methods for preparing liposomal suspensions containing active ingredients therein are generally known in the art and are disclosed in, e.g., U.S. Pat. No. 4,522,811, and Prescott, Ed., Methods in Cell Biology, Volume XIV, Academic Press, New York, N.Y. (1976), p. 33 et seq., both of which are incorporated herein by reference. Several anticancer drugs delivered in the form of liposomes are known in the art and are commercially available from Liposome Inc. of Princeton, N.J., U.S.A. It has been shown that liposomes can reduce the toxicity of the active compounds, and increase their stability.

### EXAMPLE 1

Fragments of the viral proteins selected from those in Table 1 are tested from their interaction with human Nedd4 using yeast two-hybrid system. That is, to prepare a yeast two-hybrid activation domain-Nedd4 construct, a DNA fragment encompassing the full-length coding sequence for Nedd4 is obtained by PCR from a human fetal brain cDNA library and cloned into the EcoRI/Pst1 sites of the activation domain parent plasmid GADpN2 (LEU2, CEN4, ARS1, ADH1p-SV40NLS-GAL4 (768-881)-MCS (multiple cloning site)-PGK1t, AmpR, ColE1_ori). To prepare the yeast two-hybrid DNA binding domain-PPPY-containing viral peptide construct, a DNA fragment corresponding to a contiguous amino acid sequence of a viral protein in Table 1 that spans the PPPY motif therein is obtained and is cloned into the EcoRI/Sal1 sites of the binding domain parent plasmid pGBT.Q.

To perform the yeast two-hybrid assays, yeast cells of the strain Y189 purchased from Clontech (ura3-52 his3*200 ade2-101 trp1-901 leu2-3,112 met gal4 gal80 URA3::GAL1p-lacZ) are co-transformed with the activation domain-Nedd4 construct and a binding domain-PPPY-containing viral peptide construct or the binding domain-wild type RSV p2b construct. Filter lift assays for β-Gal activity are conducted by lifting the transformed yeast colonies with filters, lysing the yeast cells by freezing and thawing, and contacting the lysed cells with X-Gal. Positive β-Gal activity indicates that the p2b wild type or PPPY-containing viral peptide interacts with Nedd4. All binding domain constructs are also tested for self-activation of β-Gal activity.

### EXAMPLE 2

A fusion protein with a GST tag fused to the RSV Gag p2b domain is recombinantly expressed and purified by chromatography. In addition, a series of fusion peptides containing a PPXY-containing short peptide according to the present invention fused to a peptidic transporter are synthesized chemically by standard peptide synthesis methods or recombinantly expressed in a standard protein expression system. The PPXY-containing short peptides are fused to a peptidic transporter such as the helix 3 of the homeodomain of Drosophila Antennapedia, HSV VP22, d-Tat₄₉₋₅₇, retro-inverso isomers of l- or d-Tat₄₉₋₅₇ (i.e., l-Tat₅₇₋₄₉ and d-Tat₅₇₋₄₉), L-arginine oligomers, and D-arginine oligomers. A number of PPXY-containing short peptides are also prepared by chemical synthesis or recombinant expression, e.g., free and unfused peptides having a sequence selected from the group of SEQ ID NOs:24-36. The peptides are purified by conventional protein purification techniques, e.g., by chromatography.

Nunc/Nalgene Maxisorp plates are incubated overnight at 4° C. or for 1-2 hrs at room temperature in 100 μl of a protein coupling solution containing purified GST-p6 and 50 mM Carbonate, pH=9.6. This allows the attachment of the GST-p6 fusion protein to the plates. Liquids in the plates are then emptied and wells filled with 400 μl/well of a blocking buffer (SuperBlock; Pierce-Endogen, Rockford, Ill.). After incubating for 1 hour at room temperature, 100 μl of a mixture containing Drosophila S2 cell lysate myc-tagged Nedd4 and a PPXY-containing short peptide is applied to the wells of the plate. This mixture is allowed to react for 2 hours at room temperature to form p2b:Nedd4 protein-protein complexes.

Plates are then washed 4×100 μl with 1×PBST solution (Invitrogen; Carlsbad, Calif.). After washing, 100 μl of 1 μg/ml solution of anti-myc monoclonal antibody (Clone 9E10; Roche Molecular Biochemicals; Indianapolis, Ind.) in 1×PBST is added to the wells of the plate to detect the myc-epitope tag on the Nedd4 protein. Plates are then washed again with 4×100 μl with 1×PBST solution and 100 μl of 1 μg/ml solution of horseradish peroxidase (HRP) conjugated Goat anti-mouse IgG (Jackson Immunoresearch Labs; West Grove, Pa.) in 1×PBST is added to the wells of the plate to detect bound mouse anti-myc antibodies. Plates are then washed again with 4×100 μl with 1×PBST solution and 100 μl of fluorescent substrate (QuantaBlu; Pierce-Endogen, Rockford, Ill.) is added to all wells. After 30 minutes, 100 μl of stop solution is added to each well to inhibit the function of HRP. Plates are then read on a Packard Fusion instrument at an excitation wavelength of 325 nm and an emission wavelength of 420 nm. The presence of fluorescent signals indicates binding of Nedd4 to the fixed GST-p2b. In contrast, the absence of fluorescent signals indicates that the PPXY-containing short peptide is capable of disrupting the interaction between Nedd4 and RSV p2b.

### EXAMPLE 3

The following examples demonstrate the anti-viral effect of the PPXY-containing short peptides tested in Example 2. The assay used is similar to the assay described by Korba and Milman, Antiviral Res., 15:217-228 (1991) and Korba and Gerin, Antiviral Res., 19:55-70 (1992), with the exception that viral DNA detection and quantification is simplified. Briefly, HepG2-2.2.15 cells are plated in 96-well microtiter plates at an initial density of 2×10⁴ cells/100 μl in DMEM medium supplemented with 10% fetal bovine serum. To promote cell adherence, the 96-well plates have been pre-coated with collagen prior to cell plating. After incubation at 37° C. in a humidified, 5% CO₂ environment for 16-24 hours, the confluent monolayer of HepG2-2.2.15 cells is washed and the medium is replaced with complete medium containing various concentrations of test compound. Every three days, the culture medium is replaced with fresh medium containing the appropriately diluted drug. Nine days following the initial administration of test compounds, the cell culture supernate is collected and clarified by centrifugation (Sorvall RT-6000D centrifuge, 1000 rpm for 5 min). Three microliters of clarified supernate is then subjected to real-time quantitative PCR using conditions described below.

Virion-associated HBV DNA present in the tissue culture supernate is PCR amplified using primers derived from HBV strain ayw. Subsequently, the PCR-amplified HBV DNA is detected in real-time (i.e., at each PCR thermocycle step) by monitoring increases in fluorescence signals that result from exonucleolytic degradation of a quenched fluorescent probe molecule following hybridization of the probe to the amplified HBV DNA. The probe molecule, designed with the aid of Primer Express™ (PE-Applied Biosystems) software, is complementary to DNA sequences present in the HBV DNA region amplified.

Routinely, 3 μl of clarified supernate is analyzed directly (without DNA extraction) in a 50 μl PCR reaction. Reagents and conditions used are per the manufacturers suggestions (PE-Applied Biosystems). For each PCR amplification, a standard curve is simultaneously generated several log dilutions of a purified 1.2 kbp HBVayw subgenomic fragment; routinely, the standard curve ranged from 1×10⁶ to 1×10 nominal copy equivalents per PCR reaction.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

TABLE 2

PPXY Motif Containing Peptides from Ebola Virus
Matrix Protein
(GenBank Accession No. AAL25816)

| | |
|---|---|
| PPEYMEAI | SEQ ID NO:39 |
| PPEYMEAIY | SEQ ID NO:40 |
| PPEYMEAIYP | SEQ ID NO:41 |
| PPEYMEAIYPV | SEQ ID NO:42 |
| PPEYMEAIYPVR | SEQ ID NO:43 |
| PPEYMEAIYPVRS | SEQ ID NO:44 |
| PPEYMEAIYPVRSN | SEQ ID NO:45 |
| PPEYMEAIYPVRSNS | SEQ ID NO:46 |
| PPEYMEAIYPVRSNST | SEQ ID NO:47 |
| PPEYMEAIYPVRSNSTI | SEQ ID NO:48 |
| PPEYMEAIYPVRSNSTIA | SEQ ID NO:49 |
| PPEYMEAIYPVRSNSTIAR | SEQ ID NO:50 |
| PPEYMEAIYPVRSNSTIARG | SEQ ID NO:51 |
| APPEYMEA | SEQ ID NO:52 |
| APPEYMEAI | SEQ ID NO:53 |
| APPEYMEAIY | SEQ ID NO:54 |
| APPEYMEAIYP | SEQ ID NO:55 |
| APPEYMEAIYPV | SEQ ID NO:56 |
| APPEYMEAIYPVR | SEQ ID NO:57 |
| APPEYMEAIYPVRS | SEQ ID NO:58 |
| APPEYMEAIYPVRSN | SEQ ID NO:59 |
| APPEYMEAIYPVRSNS | SEQ ID NO:60 |
| APPEYMEAIYPVRSNST | SEQ ID NO:61 |
| APPEYMEAIYPVRSNSTI | SEQ ID NO:62 |
| APPEYMEAIYPVRSNSTIA | SEQ ID NO:63 |
| APPEYMEAIYPVRSNSTIAR | SEQ ID NO:64 |
| TAPPEYME | SEQ ID NO:65 |
| TAPPEYMEA | SEQ ID NO:66 |
| TAPPEYMEAI | SEQ ID NO:67 |
| TAPPEYMEAIY | SEQ ID NO:68 |
| TAPPEYMEAIYP | SEQ ID NO:69 |
| TAPPEYMEAIYPV | SEQ ID NO:70 |
| TAPPEYMEAIYPVR | SEQ ID NO:71 |
| TAPPEYMEAIYPVRS | SEQ ID NO:72 |
| TAPPEYMEAIYPVRSN | SEQ ID NO:73 |
| TAPPEYMEAIYPVRSNS | SEQ ID NO:74 |
| TAPPEYMEAIYPVRSNST | SEQ ID NO:75 |
| TAPPEYMEAIYPVRSNSTI | SEQ ID NO:76 |
| TAPPEYMEAIYPVRSNSTIA | SEQ ID NO:77 |
| PTAPPEYM | SEQ ID NO:78 |
| PTAPPEYME | SEQ ID NO:79 |
| PTAPPEYMEA | SEQ ID NO:80 |
| PTAPPEYMEAI | SEQ ID NO:81 |
| PTAPPEYMEAIY | SEQ ID NO:82 |
| PTAPPEYMEAIYP | SEQ ID NO:83 |
| PTAPPEYMEAIYPV | SEQ ID NO:84 |
| PTAPPEYMEAIYPVR | SEQ ID NO:85 |
| PTAPPEYMEAIYPVRS | SEQ ID NO:86 |
| PTAPPEYMEAIYPVRSN | SEQ ID NO:87 |
| PTAPPEYMEAIYPVRSNS | SEQ ID NO:88 |
| PTAPPEYMEAIYPVRSNST | SEQ ID NO:89 |
| PTAPPEYMEAIYPVRSNSTI | SEQ ID NO:90 |
| LPTAPPEY | SEQ ID NO:91 |
| LPTAPPEYM | SEQ ID NO:92 |
| LPTAPPEYME | SEQ ID NO:93 |
| LPTAPPEYMEA | SEQ ID NO:94 |
| LPTAPPEYMEAI | SEQ ID NO:95 |
| LPTAPPEYMEAIY | SEQ ID NO:96 |
| LPTAPPEYMEAIYP | SEQ ID NO:97 |
| LPTAPPEYMEAIYPV | SEQ ID NO:98 |
| LPTAPPEYMEALYPVR | SEQ ID NO:99 |
| LPTAPPEYMEAIYPVRS | SEQ ID NO:100 |
| LPTAPPEYMEAIYPVRSN | SEQ ID NO:101 |
| LPTAPPEYMEAIYPVRSNS | SEQ ID NO:102 |
| LPTAPPEYMEAIYPVRSNST | SEQ ID NO:103 |
| ILPTAPPEY | SEQ ID NO:104 |
| ILPTAPPEYM | SEQ ID NO:105 |
| ILPTAPPEYME | SEQ ID NO:106 |
| ILPTAPPEYMEA | SEQ ID NO:107 |
| ILPTAPPEYMEAI | SEQ ID NO:108 |
| ILPTAPPEYMEAIY | SEQ ID NO:109 |
| ILPTAPPEYMEAIYP | SEQ ID NO:110 |
| ILPTAPPEYMEAIYPV | SEQ ID NO:111 |
| ILPTAPPEYMEAIYPVR | SEQ ID NO:112 |
| ILPTAPPEYMEAIYPVRS | SEQ ID NO:113 |
| ILPTAPPEYMEAIYPVRSN | SEQ ID NO:114 |
| ILPTAPPEYMEAIYPVRSNS | SEQ ID NO:115 |
| VILPTAPPEY | SEQ ID NO:116 |
| VILPTAPPEYM | SEQ ID NO:117 |
| VILPTAPPEYME | SEQ ID NO:118 |
| VILPTAPPEYMEA | SEQ ID NO:119 |
| VILPTAPPEYMEAI | SEQ ID NO:120 |
| VILPTAPPEYMEAIY | SEQ ID NO:121 |
| VILPTAPPEYMEAIYP | SEQ ID NO:122 |
| VILPTAPPEYMEAIYPV | SEQ ID NO:123 |
| VILPTAPPEYMEAIYPVR | SEQ ID NO:124 |
| VILPTAPPEYMEAIYPVRS | SEQ ID NO:125 |
| VILPTAPPEYMEAIYPVRSN | SEQ ID NO:126 |
| RVTLPTAPPEY | SEQ ID NO:127 |
| RVLLPTAPPEYM | SEQ ID NO:128 |
| RVILPTAPPEYME | SEQ ID NO:129 |
| RVILPTAPPEYMEA | SEQ ID NO:130 |
| RVILPTAPPEYMEAI | SEQ ID NO:131 |
| RVILPTAPPEYMEAIY | SEQ ID NO:132 |
| RVILPTAPPEYMEAIYP | SEQ ID NO:133 |
| RVILPTAPPEYMEAIYPV | SEQ ID NO:134 |
| RVILPTAPPEYMEAIYPVR | SEQ ID NO:135 |
| RVILPTAPPEYMEAIYPVRS | SEQ ID NO:136 |
| RRVILPTAPPEY | SEQ ID NO:137 |
| RRVILPTAPPEYM | SEQ ID NO:138 |
| RRVILPTAPPEYME | SEQ ID NO:139 |
| RRVILPTAPPEYMEA | SEQ ID NO:140 |
| RRVILPTAPPEYMEAI | SEQ ID NO:141 |
| RRVILPTAPPEYMEAIY | SEQ ID NO:142 |
| RRVILPTAPPEYMEAIYP | SEQ ID NO:143 |
| RRVILPTAPPEYMEAIYPV | SEQ ID NO:144 |
| RRVILPTAPPEYMEAIYPVR | SEQ ID NO:145 |
| MRRVILPTAPPEY | SEQ ID NO:146 |
| MRRVILPTAPPEYM | SEQ ID NO:147 |
| MRRVILPTAPPEYME | SEQ ID NO:148 |
| MRRVILPTAPPEYMEA | SEQ ID NO:149 |
| MRRVILPTAPPEYMEAI | SEQ ID NO:150 |
| MRRVILPTAPPEYMEAIY | SEQ ID NO:151 |
| MRRVILPTAPPEYMEAIYP | SEQ ID NO:152 |
| MRRVILPTAPPEYMEAIYPV | SEQ ID NO:153 |

TABLE 3

PPXY Motif Containing Peptides from Marburg Virus
VP40 Protein
(GenBank Accession No. NP_042027)

| | |
|---|---|
| PPPYADHG | SEQ ID NO:154 |
| PPPYADHGA | SEQ ID NO:155 |
| PPPYADHGAN | SEQ ID NO:156 |
| PPPYADHGANQ | SEQ ID NO:157 |
| PPPYADHGANQL | SEQ ID NO:158 |
| PPPYADHGANQLI | SEQ ID NO:159 |
| PPPYADHGANQLIP | SEQ ID NO:160 |
| PPPYADHGANQLIPA | SEQ ID NO:161 |
| PPPYADHGANQLIPAD | SEQ ID NO:162 |
| PPPYADHGANQLIPADQ | SEQ ID NO:163 |
| PPPYADHGANQLIPADQL | SEQ ID NO:164 |
| PPPYADHGANQLIPADQLS | SEQ ID NO:165 |
| PPPYADHGANQLIPADQLSN | SEQ ID NO:166 |
| NPPPYADH | SEQ ID NO:167 |
| NPPPYADHG | SEQ ID NO:168 |
| NPPPYADHGA | SEQ ID NO:169 |
| NFPPYADHGAN | SEQ ID NO:170 |
| NPPPYADHGANQ | SEQ ID NO:171 |
| NPPPYADHGANQL | SEQ ID NO:172 |
| NPPPYADHGANQLI | SEQ ID NO:173 |
| NPPPYADHGANQLIP | SEQ ID NO:174 |
| NPPPYADHGANQLIPA | SEQ ID NO:175 |
| NPPPYADHGANQLIPAD | SEQ ID NO:176 |
| NPPPYADHGANQLIPADQ | SEQ ID NO:177 |
| NPPPYADHGANQLIPADQL | SEQ ID NO:178 |
| NIPPYADHGANQLIPADQLS | SEQ ID NO:179 |
| LNPPPYAD | SEQ ID NO:180 |
| LNPPPYADH | SEQ ID NO:181 |
| LNPPPYADHG | SEQ ID NO:182 |
| LNPPPYADHGA | SEQ ID NO:183 |
| LNPPPYADHGAN | SEQ ID NO:184 |
| LNPPPYADHGANQ | SEQ ID NO:185 |
| LNPPPYADHGANQL | SEQ ID NO:186 |
| LNPPPYADHGANQLI | SEQ ID NO:187 |
| LNPPPYADHGANQLIP | SEQ ID NO:188 |
| LNPPPYADHGANQLIPA | SEQ ID NO:189 |
| LNPPPYADHGANQLIPAD | SEQ ID NO:190 |
| LNPPPYADHGANQLIPADQ | SEQ ID NO:191 |
| LNPPPYADHGANQLIPADQL | SEQ ID NO:192 |
| YLNPPPYA | SEQ ID NO:193 |
| YLNPPPYAD | SEQ ID NO:194 |
| YLNPPPYADH | SEQ ID NO:195 |
| YLNPPPYADHG | SEQ ID NO:196 |
| YLNPPPYADHGA | SEQ ID NO:197 |
| YLNPPPYADHGAN | SEQ ID NO:198 |
| YLNPPPYADHGANQ | SEQ ID NO:199 |
| YLNPPPYADHGANQL | SEQ ID NO:200 |
| YLNPPPYADHGANQLI | SEQ ID NO:201 |
| YLNPPPYADHGANQLIP | SEQ ID NO:202 |
| YLNPPPYADHGANQLIPA | SEQ ID NO:203 |
| YLNPPPYADHGANQLIPAD | SEQ ID NO:204 |
| YLNPPPYADHGANQLTPADQ | SEQ ID NO:205 |
| QYLNPPPY | SEQ ID NO:206 |
| QYLNPPPYA | SEQ ID NO:207 |
| QYLNPPPYAD | SEQ ID NO:208 |
| QYLNPPPYADH | SEQ ID NO:209 |
| QYLNPPPYADHG | SEQ ID NO:210 |
| QYLNPPPYADHGA | SEQ ID NO:211 |
| QYLNPPPYADHGAN | SEQ ID NO:212 |
| QYLNPPPYADHGANQ | SEQ ID NO:213 |
| QYLNPPPYADHGANQL | SEQ ID NO:214 |
| QYLNPPPYADHGANQLI | SEQ ID NO:215 |
| QYLNPPPYADHGANQLIP | SEQ ID NO:216 |
| QYLNPPPYADHGANQLIPA | SEQ ID NO:217 |
| QYLNPPPYADHGANQLIPAD | SEQ ID NO:218 |
| MQYLNPPPY | SEQ ID NO:219 |
| MQYLNPPPYA | SEQ ID NO:220 |
| MQYLNPPPYAD | SEQ ID NO:221 |
| MQYLNPPPYADH | SEQ ID NO:222 |
| MQYLNPPPYADHG | SEQ ID NO:223 |
| MQYLNPPPYADHGA | SEQ ID NO:224 |
| MQYLNPPPYADHGAN | SEQ ID NO:225 |
| MQYLNPPPYADHGANQ | SEQ ID NO:226 |
| MQYLNPPPYADHGANQL | SEQ ID NO:227 |
| MQYLNPPPYADHGANQLI | SEQ ID NO:228 |
| MQYLNPPPYADHGANQLIP | SEQ ID NO:229 |
| MQYLNPPPYADHGANQLIPA | SEQ ID NO:230 |
| YMQYLNPPPY | SEQ ID NO:231 |
| YMQYLNPPPYA | SEQ ID NO:232 |
| YMQYLNPPPYAD | SEQ ID NO:233 |
| YMQYLNPPPYADH | SEQ ID NO:234 |
| YMQYLNPPPYADHG | SEQ ID NO:235 |
| YMQYLNPPPYADHGA | SEQ ID NO:236 |
| YMQYLNPPPYADHGAN | SEQ ID NO:237 |
| YMQYLNPPPYADHGANQ | SEQ ID NO:238 |
| YMQYLNPPPYADHGANQL | SEQ ID NO:239 |
| YMQYLNPPPYADHGANQLI | SEQ ID NO:240 |
| YMQYLNPPPYADHGANQLIP | SEQ ID NO:241 |
| TYMQYLNPPPY | SEQ ID NO:242 |
| TYMQYLNPPPYA | SEQ ID NO:243 |
| TYMQYLNPPPYAD | SEQ ID NO:244 |
| TYMQYLNPPPYADH | SEQ ID NO:245 |
| TYMQYLNPPPYADHG | SEQ ID NO:246 |
| TYMQYLNPPPYADHGA | SEQ ID NO:247 |
| TYMQYLNPPPYADHGAN | SEQ ID NO:248 |
| TYMQYLNPPPYADHGANQ | SEQ ID NO:249 |
| TYMQYLNPPPYADHGANQL | SEQ ID NO:250 |
| TYMQYLNPPPYADHGANQLI | SEQ ID NO:251 |
| NTYMQYLNPPPY | SEQ ID NO:252 |
| NTYMQYLNPPPYA | SEQ ID NO:253 |
| NTYMQYLNPPPYAD | SEQ ID NO:254 |
| NTYMQYLNPPPYADH | SEQ ID NO:255 |
| NTYMQYLNPPPYADHG | SEQ ID NO:256 |
| NTYMQYLNPPPYADHGA | SEQ ID NO:257 |
| NTYMQYLNPPPYADHGAN | SEQ ID NO:258 |
| NTYMQYLNPPPYADHGANQ | SEQ ID NO:259 |
| NTYMQYLNPPPYADHGANQL | SEQ ID NO:260 |
| YNTYMQYLNPPPY | SEQ ID NO:261 |
| YNTYMQYLNPPPYA | SEQ ID NO:262 |
| YNTYMQYLNPPPYAD | SEQ ID NO:263 |
| YNTYMQYLNPPPYADH | SEQ ID NO:264 |
| YNTYMQYLNPPPYADHG | SEQ ID NO:265 |
| YNTYMQYLNPPPYADHGA | SEQ ID NO:266 |
| YNTYMQYLNPPPYADHGAN | SEQ ID NO:267 |
| YNTYMQYLNPPPYADHGANQ | SEQ ID NO:268 |
| NYNTYMQYLNPPPY | SEQ ID NO:269 |
| NYNTYMQYLNPPPYA | SEQ ID NO:270 |
| NYNTYMQYLNPPPYAD | SEQ ID NO:271 |
| NYNTYMQYLNPPPYADH | SEQ ID NO:272 |
| NYNTYMQYLNPPPYADHG | SEQ ID NO:273 |
| NYNTYMQYLNPPPYADHGA | SEQ ID NO:274 |
| NYNTYMQYLNPPPYADHGAN | SEQ ID NO:275 |
| SNYNTYMQYLNPPPY | SEQ ID NO:276 |
| SNYNTYMQYLNPPPYA | SEQ ID NO:277 |
| SNYNTYMQYLNPPPYAD | SEQ ID NO:278 |
| SNYNTYMQYLNPPPYADH | SEQ ID NO:279 |
| SNYNTYMQYLNPPPYADHG | SEQ ID NO:280 |
| SNYNTYMQYLNPPPYADHGA | SEQ ID NO:281 |
| SSNYNTYMQYLNPPPY | SEQ ID NO:282 |
| SSNYNTYMQYLNPPPYA | SEQ ID NO:283 |
| SSNYNTYMQYLNPPPYAD | SEQ ID NO:284 |
| SSNYNTYMQYLNPPPYADH | SEQ ID NO:285 |
| SSNYNTYMQYLNPPPYADHG | SEQ ID NO:286 |
| SSSNYNTYMQYLNPPPY | SEQ ID NO:287 |
| SSSNYNTYMQYLNPPPYA | SEQ ID NO:288 |
| SSSNYNTYMQYLNPPPYAD | SEQ ID NO:289 |
| SSSNYNTYMQYLNPPPYADH | SEQ ID NO:290 |
| ASSSNYNTYMQYLNPPPY | SEQ ID NO:291 |
| ASSSNYNTYMQYLNPPPYA | SEQ ID NO:292 |
| ASSSNYNTYMQYLNPPPYAD | SEQ ID NO:293 |
| MASSSNYNTYMQYLNPPPY | SEQ ID NO:294 |
| MASSSNYNTYMQYLNPPPYA | SEQ ID NO:295 |

TABLE 4

PPXY Motif Containing Peptides from Vesicular
Stomatitis Virus Matrix Protein
(GenBank Accession No. P04876)

| | |
|---|---|
| PPPYEEDT | SEQ ID NO:296 |
| PPPYEEDTS | SEQ ID NO:297 |
| PPPYEEDTSM | SEQ ID NO:298 |
| PPPYEEDTSME | SEQ ID NO:299 |
| PPPYEEDTSMEY | SEQ ID NO:300 |
| PPPYEEDTSMEYA | SEQ ID NO:301 |
| PPPYEEDTSMEYAP | SEQ ID NO:302 |
| PPPYEEDTSMEYAPS | SEQ ID NO:303 |
| PPPYEEDTSMEYAPSA | SEQ ID NO:304 |
| PPPYEEDTSMEYAPSAP | SEQ ID NO:305 |
| PPPYEEDTSMEYAPSAPI | SEQ ID NO:306 |
| PPPYEEDTSMEYAPSAPID | SEQ ID NO:307 |
| PPPYEEDTSMEYAPSAPIDK | SEQ ID NO:308 |
| APPPYEED | SEQ ID NO:309 |
| APPPYEEDT | SEQ ID NO:310 |
| APPPYEEDTS | SEQ ID NO:311 |
| APPPYEEDTSM | SEQ ID NO:312 |
| APPPYEEDTSME | SEQ ID NO:313 |
| APPPYEEDTSMEY | SEQ ID NO:314 |
| APPPYEEDTSMEYA | SEQ ID NO:315 |
| APPPYEEDTSMEYAP | SEQ ID NO:316 |
| APPPYEEDTSMEYAPS | SEQ ID NO:317 |
| APPPYEEDTSMEYAPSA | SEQ ID NO:318 |
| APPPYEEDTSMEYAPSAP | SEQ ID NO:319 |
| APPPYEEDTSMEYAPSAPI | SEQ ID NO:320 |
| APPPYEEDTSMEYAPSAPID | SEQ ID NO:321 |
| IAPPPYEE | SEQ ID NO:322 |
| IAPPPYEED | SEQ ID NO:323 |
| IAPPPYEEDT | SEQ ID NO:324 |
| IAPPPYEEDTS | SEQ ID NO:325 |
| IAPPPYEEDTSM | SEQ ID NO:326 |
| IAPPPYEEDTSME | SEQ ID NO:327 |
| IAPPPYEEDTSMEY | SEQ ID NO:328 |
| IAPPPYEEDTSMEYA | SEQ ID NO:329 |
| IAPPPYEEDTSMEYAP | SEQ ID NO:330 |
| IAPPPYEEDTSMEYAPS | SEQ ID NO:331 |
| IAPPPYEEDTSMEYAPSA | SEQ ID NO:332 |
| IAPPPYEEDTSMEYAPSAP | SEQ ID NO:333 |
| IAPPPYEEDTSMEYAPSAPI | SEQ ID NO:334 |
| GIAPPPYE | SEQ ID NO:335 |
| GIAPPPYEE | SEQ ID NO:336 |
| GIAPPPYEED | SEQ ID NO:337 |
| GIAPPPYEEDT | SEQ ID NO:338 |
| GIAPPPYEEDTS | SEQ ID NO:339 |
| GIAPPPYEEDTSM | SEQ ID NO:340 |
| GIAPPPYEEDTSME | SEQ ID NO:341 |
| GIAPPPYEEDTSMEY | SEQ ID NO:342 |
| GIAPPPYEEDTSMEYA | SEQ ID NO:343 |
| GIAPPPYEEDTSMEYAP | SEQ ID NO:344 |
| GIAPPPYEEDTSMEYAPS | SEQ ID NO:345 |
| GIAPPPYEEDTSMEYAPSA | SEQ ID NO:346 |
| GIAPPPYEEDTSMEYAPSAP | SEQ ID NO:347 |
| LGIAPPPY | SEQ ID NO:348 |
| LGIAPPPYE | SEQ ID NO:349 |
| LGIAPPPYEE | SEQ ID NO:350 |
| LGIAPPPYEED | SEQ ID NO:351 |
| LGIAPPPYEEDT | SEQ ID NO:352 |
| LGIAPPPYEEDTS | SEQ ID NO:353 |
| LGIAPPPYEEDTSM | SEQ ID NO:354 |
| LGIAPPPYEEDTSME | SEQ ID NO:355 |
| LGIAPPPYEEDTSMEY | SEQ ID NO:356 |
| LGIAPPPYEEDTSMEYA | SEQ ID NO:357 |
| LGIAPPPYEEDTSMEYAP | SEQ ID NO:358 |
| LGIAPPPYEEDTSMEYAPS | SEQ ID NO:359 |
| LGIAPPPYEEDTSMEYAPSA | SEQ ID NO:360 |
| KLGIAPPPY | SEQ ID NO:361 |
| KLGIAPPPYE | SEQ ID NO:362 |
| KLGIAPPPYEE | SEQ ID NO:363 |
| KLGIAPPPYEED | SEQ ID NO:364 |
| KLGIAPPPYEEDT | SEQ ID NO:365 |
| KLGIAPPPYEEDTS | SEQ ID NO:366 |
| KLGIAPPPYEEDTSM | SEQ ID NO:367 |
| KLGLAPPPYEEDTSME | SEQ ID NO:368 |
| KLGIAPPPYEEDTSMEY | SEQ ID NO:369 |
| KLGIAPPPYEEDTSMEYA | SEQ ID NO:370 |
| KLGIAPPPYEEDTSMEYAP | SEQ ID NO:371 |
| KLGIAPPPYEEDTSMEYAPS | SEQ ID NO:372 |
| KKLGIAPPPY | SEQ ID NO:373 |
| KKLGIAPPPYE | SEQ ID NO:374 |
| KKLGLAPPPYEE | SEQ ID NO:375 |
| KKLGIAPPPYEED | SEQ ID NO:376 |
| KKLGIAPPPYEEDT | SEQ ID NO:377 |
| KKLGIAPPPYEEDTS | SEQ ID NO:378 |
| KKLGIAPPPYEEDTSM | SEQ ID NO:379 |
| KKLGIAPPPYEEDTSME | SEQ ID NO:380 |
| KKLGIAPPPYEEDTSMEY | SEQ ID NO:381 |
| KKLGIAPPPYEEDTSMEYA | SEQ ID NO:382 |
| KKLGIAPPPYEEDTSMEYAP | SEQ ID NO:383 |
| SKKLGIAPPPY | SEQ ID NO:384 |
| SKKLGIAPPPYE | SEQ ID NO:385 |
| SKKLGIAPPPYEE | SEQ ID NO:386 |
| SKKLGIAPPPYEED | SEQ ID NO:387 |
| SKKLGIAPPPYEEDT | SEQ ID NO:388 |
| SKKLGIAPPPYEEDTS | SEQ ID NO:389 |
| SKKLGIAPPPYEEDTSM | SEQ ID NO:390 |
| SKKLGIAPPPYEEDTSME | SEQ ID NO:391 |
| SKKLGIAPPPYEEDTSMEY | SEQ ID NO:392 |
| SKKLGIAPPPYEEDTSMEYA | SEQ ID NO:393 |
| KSKKLGIAPPPY | SEQ ID NO:394 |
| KSKKLGIAPPPYE | SEQ ID NO:395 |
| KSKKLGIAPPPYEE | SEQ ID NO:396 |
| KSKKLGIAPPPYEED | SEQ ID NO:397 |
| KSKKLGIAPPPYEEDT | SEQ ID NO:398 |
| KSKKLGIAPPPYEEDTS | SEQ ID NO:399 |
| KSKKLGIAPPPYEEDTSM | SEQ ID NO:400 |
| KSKKLGIAPPPYEEDTSME | SEQ ID NO:401 |
| KSKKLGIAPPPYEEDTSMEY | SEQ ID NO:402 |
| KKSKKLGIAPPPY | SEQ ID NO:403 |
| KKSKKLGIAPPPYE | SEQ ID NO:404 |
| KKSKKLGIAPPPYEE | SEQ ID NO:405 |
| KKSKKLGIAPPPYEED | SEQ ID NO:406 |
| KKSKKLGIAPPPYEEDT | SEQ ID NO:407 |
| KKSKKLGIAPPPYEEDTS | SEQ ID NO:408 |
| KKSKKLGIAPPPYEEDTSM | SEQ ID NO:409 |
| KKSKKLGIAPPPYEEDTSME | SEQ ID NO:410 |
| GKKSKKLGIAPPPY | SEQ ID NO:411 |
| GKKSKKLGIAPPPYE | SEQ ID NO:412 |
| GKKSKKLGIAPPPYEE | SEQ ID NO:413 |
| GKKSKKLGIAPPPYEED | SEQ ID NO:414 |
| GKKSKKLGIAPPPYEEDT | SEQ ID NO:415 |
| GKKSKKLGIAPPPYEEDTS | SEQ ID NO:416 |
| GKKSKKLGIAPPPYEEDTSM | SEQ ID NO:417 |
| KGKKSKKLGIAPPPY | SEQ ID NO:418 |
| KGKKSKKLGIAPPPYE | SEQ ID NO:419 |
| KGKKSKKLGIAPPPYEE | SEQ ID NO:420 |
| KGKKSKKLGIAPPPYEED | SEQ ID NO:421 |
| KGKKSKKLGLAPPPYEEDT | SEQ ID NO:422 |
| KGKKSKKLGIAPPPYEEDTS | SEQ ID NO:423 |
| GKGKKSKKLGIAPPPY | SEQ ID NO:424 |
| GKGKKSKKLGIAPPPYE | SEQ ID NO:425 |
| GKGKKSKKLGIAPPPYEE | SEQ ID NO:426 |
| GKGKKSKKLGIAPPPYEED | SEQ ID NO:427 |
| GKGKKSKKLGIAPPPYEEDT | SEQ ID NO:428 |
| KGKGKKSKKLGIAPPPY | SEQ ID NO:429 |
| KGKGKKSKKLGIAPPPYE | SEQ ID NO:430 |
| KGKGKKSKKLGIAPPPYEE | SEQ ID NO:431 |
| KGKGKKSKKLGIAPPPYEED | SEQ ID NO:432 |
| LKGKGKKSKKLGIAPPPY | SEQ ID NO:433 |
| LKGKGKKSKKLGIAPPPYE | SEQ ID NO:434 |
| LKGKGKKSKKLGIAPPPYEE | SEQ ID NO:435 |
| GLKGKGKKSKKLGIAPPPY | SEQ ID NO:436 |
| GLKGKGKKSKKLGIAPPPYE | SEQ ID NO:437 |
| LGLKGKGKKSKKLGIAPPPY | SEQ ID NO:438 |

TABLE 5

PPPY Motif Containing Peptides from Rous Sarcoma
Virus GAG Protein
(Genbank Accession No. AAA19608)

| | |
|---|---|
| PPPYVGSG | SEQ ID NO:439 |
| PPPYVGSGL | SEQ ID NO:440 |
| PPPYVGSGLY | SEQ ID NO:441 |
| PPPYVGSGLYP | SEQ ID NO:442 |
| PPPYVGSGLYPS | SEQ ID NO:443 |
| PPPYVGSGLYPSL | SEQ ID NO:444 |
| PPPYVGSGLYPSLA | SEQ ID NO:445 |
| PPPYVGSGLYPSLAG | SEQ ID NO:446 |
| PPPYVGSGLYPSLAGV | SEQ ID NO:447 |
| PPPYVGSGLYPSLAGVG | SEQ ID NO:448 |
| PPPYVGSGLYPSLAGVGE | SEQ ID NO:449 |
| PPPYVGSGLYPSLAGVGEQ | SEQ ID NO:450 |
| PPPYVGSGLYPSLAGVGEQQ | SEQ ID NO:451 |
| PPPPYVGS | SEQ ID NO:452 |
| PPPPYVGSG | SEQ ID NO:453 |
| PPPPYVGSGL | SEQ ID NO:454 |
| PPPPYVGSGLY | SEQ ID NO:455 |
| PPPPYVGSGLYP | SEQ ID NO:456 |
| PPPPYVGSGLYPS | SEQ ID NO:457 |
| PPPPYVGSGLYPSL | SEQ ID NO:458 |
| PPPPYVGSGLYPSLA | SEQ ID NO:459 |
| PPPPYVGSGLYPSLAG | SEQ ID NO:460 |
| PPPPYVGSGLYPSLAGV | SEQ ID NO:461 |
| PPPPYVGSGLYPSLAGVG | SEQ ID NO:462 |
| PPPPYVGSGLYPSLAGVGE | SEQ ID NO:463 |
| PPPPYVGSGLYPSLAGVGEQ | SEQ ID NO:464 |
| APPPPYVG | SEQ ID NO:465 |
| APPPPYVGS | SEQ ID NO:466 |
| APPPPYVGSG | SEQ ID NO:467 |
| APPPPYVGSGL | SEQ ID NO:468 |
| APPPPYVGSGLY | SEQ ID NO:469 |
| APPPPYVGSGLYP | SEQ ID NO:470 |
| APPPPYVGSGLYPS | SEQ ID NO:471 |
| APPPPYVGSGLYPSL | SEQ ID NO:472 |
| APPPPYVGSGLYPSLA | SEQ ID NO:473 |
| APPPPYVGSGLYPSLAG | SEQ ID NO:474 |
| APPPPYVGSGLYPSLAGV | SEQ ID NO:475 |
| APPPPYVGSGLYPSLAGVG | SEQ ID NO:476 |
| APPPPYVGSGLYPSLAGVGE | SEQ ID NO:477 |
| SAPPPPYV | SEQ ID NO:478 |
| SAPPPPYVG | SEQ ID NO:479 |
| SAPPPPYVGS | SEQ ID NO:480 |
| ATATASAPPPPYVGSGL | SEQ ID NO:523 |
| ATATASAPPPPYVGSGLY | SEQ ID NO:524 |
| ATATASAPPPPYVGSGLYP | SEQ ID NO:525 |
| ATASAPPPPYVGSGLYPSLA | SEQ ID NO:526 |
| TATASAPPPPY | SEQ ID NO:527 |
| TATASAPPPPYV | SEQ ID NO:528 |
| TATASAPPPPYVG | SEQ ID NO:529 |
| TATASAPPPPYVGS | SEQ ID NO:530 |
| TATASAPPPPYVGSG | SEQ ID NO:531 |
| TATASAPPPPYVGSGL | SEQ ID NO:532 |
| TATASAPPPPYVGSGLY | SEQ ID NO:533 |
| TATASAPPPPYVGSGLYP | SEQ ID NO:534 |
| TATASAPPPPYVGSGLYPS | SEQ ID NO:535 |
| TATASAPPPPYVGSGLYPSL | SEQ ID NO:536 |
| ATATASAPPPPY | SEQ ID NO:537 |
| ATATASAPPPPYV | SEQ ID NO:538 |
| ATATASAPPPPYVG | SEQ ID NO:539 |
| ATATASAPPPPYVGS | SEQ ID NO:540 |
| ATATASAPPPPYVGSG | SEQ ID NO:541 |
| ATATASAPPPPYVGSGL | SEQ ID NO:542 |
| ATATASAPPPPYVGSGLY | SEQ ID NO:543 |
| ATATASAPPPPYVGSGLYP | SEQ ID NO:544 |
| ATATASAPPPPYVGSGLYPS | SEQ ID NO:545 |
| CATATASAPPPPY | SEQ ID NO:546 |
| CATATASAPPPPYV | SEQ ID NO:547 |
| CATATASAPPPPYVG | SEQ ID NO:548 |
| CATATASAPPPPYVGS | SEQ ID NO:549 |
| CATATASAPPPPYVGSG | SEQ ID NO:550 |
| CATATASAPPPPYVGSGL | SEQ ID NO:551 |
| CATATASAPPPPYVGSGLY | SEQ ID NO:552 |
| CATATASAPPPPYVGSGLYP | SEQ ID NO:553 |
| NCATATASAPPPPY | SEQ ID NO:554 |
| NCATATASAPPPPYV | SEQ ID NO:555 |
| NCATATASAPPPPYVG | SEQ ID NO:556 |
| NCATATASAPPPPYVGS | SEQ ID NO:557 |
| NCATATASAPPPPYVGSG | SEQ ID NO:558 |
| NCATATASAPPPPYVGSGL | SEQ ID NO:559 |
| NCATATASAPPPPYVGSGLY | SEQ ID NO:560 |
| CNCATATASAPPPPY | SEQ ID NO:561 |
| CNCATATASAPPPPYV | SEQ ID NO:562 |
| CNCATATASAPPPPYVG | SEQ ID NO:563 |
| CNCATATASAPPPPYVGS | SEQ ID NO:564 |
| CNCATATASAPPPPYVGSG | SEQ ID NO:565 |
| CNCATATASAPPPPYVGSGL | SEQ ID NO:566 |
| GCNCATATASAPPPPY | SEQ ID NO:567 |
| GCNCATATASAPPPPYV | SEQ ID NO:568 |
| GCNCATATASAPPPPYVG | SEQ ID NO:569 |
| SAPPPPYVGSG | SEQ ID NO:481 |
| SAPPPPYVGSGL | SEQ ID NO:482 |
| SAPPPPYVGSGLY | SEQ ID NO:483 |
| SAPPPPYVGSGLYP | SEQ ID NO:484 |
| SAPPPPYVGSGLYPS | SEQ ID NO:485 |
| SAPPPPYVGSGLYPSL | SEQ ID NO:486 |
| SAPPPPYVGSGLYPSLA | SEQ ID NO:487 |
| SAPPPPYVGSGLYPSLAG | SEQ ID NO:488 |
| SAPPPPYVGSGLYPSLAGV | SEQ ID NO:489 |
| SAPPPPYVGSGLYPSLAGVG | SEQ ID NO:490 |
| ASAPPPPY | SEQ ID NO:491 |
| ASAPPPPYV | SEQ ID NO:492 |
| ASAPPPPYVG | SEQ ID NO:493 |
| ASAPPPPYVGS | SEQ ID NO:494 |
| ASAPPPPYVGSG | SEQ ID NO:495 |
| ASAPPPPYVGSGL | SEQ ID NO:496 |
| ASAPPPPYVGSGLY | SEQ ID NO:497 |
| ASAPPPPYVGSGLYP | SEQ ID NO:498 |
| ASAPPPPYVGSGLYPS | SEQ ID NO:499 |
| ASAPPPPYVGSGLYPSL | SEQ ID NO:500 |
| ASAPPPPYVGSGLYPSLA | SEQ ID NO:501 |
| ASAPPPPYVGSGLYPSLAG | SEQ ID NO:502 |
| ASAPPPPYVGSGLYPSLAGV | SEQ ID NO:503 |
| TASAPPPPY | SEQ ID NO:504 |
| TASAPPPPYV | SEQ ID NO:505 |
| TASAPPPPYVG | SEQ ID NO:506 |
| TASAPPPPYVGS | SEQ ID NO:507 |
| TASAPPPPYVGSG | SEQ ID NO:508 |
| TASAPPPPYVGSGL | SEQ ID NO:509 |
| TASAPPPPYVGSGLY | SEQ ID NO:510 |
| TASAPPPPYVGSGLYP | SEQ ID NO:511 |
| TASAPPPPYVGSGLYPS | SEQ ID NO:512 |
| TASAPPPPYVGSGLYPSL | SEQ ID NO:513 |
| TASAPPPPYVGSGLYPSLA | SEQ ID NO:514 |
| TASAPPPPYVGSGLYPSLAG | SEQ ID NO:515 |
| ATASAPPPPY | SEQ ID NO:516 |
| ATASAPPPPYV | SEQ ID NO:517 |
| ATASAPPPPYVG | SEQ ID NO:518 |
| ATASAPPPPYVGS | SEQ ID NO:519 |
| ATASAPPPPYVGSG | SEQ ID NO:520 |
| ATASAPPPPYVGSGL | SEQ ID NO:521 |
| ATASAPPPPYVGSGLY | SEQ ID NO:522 |
| GCNCATATASAPPPPYVGS | SEQ ID NO:570 |
| GCNCATATASAPPPPYVGSG | SEQ ID NO:571 |
| VGCNCATATASAPPPPY | SEQ ID NO:572 |
| VGCNCATATASAPPPPYV | SEQ ID NO:573 |
| VGCNCATATASAPPPPYVG | SEQ ID NO:574 |
| VGCNCATATASAPPPPYVGS | SEQ ID NO:575 |
| AVGCNCATATASAPPPPY | SEQ ID NO:576 |
| AVGCNCATATASAPPPPYV | SEQ ID NO:577 |
| AVGCNCATATASAPPPPYVG | SEQ ID NO:578 |
| TAVGCNCATATASAPPPPY | SEQ ID NO:579 |
| TAVGCNCATATASAPPPPYV | SEQ ID NO:580 |
| GTAVGCNCATATASAPPPPY | SEQ ID NO:581 |
| PPEYMEAI | SEQ ID NO:39 |
| PPEYMEAIY | SEQ ID NO:40 |
| PPEYMEAIYP | SEQ ID NO:41 |
| PPEYMEAIYPV | SEQ ID NO:42 |
| PPEYMEAIYPVR | SEQ ID NO:43 |
| PPEYMEAIYPVRS | SEQ ID NO:44 |
| PPEYMEAIYPVRSN | SEQ ID NO:45 |
| PPEYMEAIYPVRSNS | SEQ ID NO:46 |
| PPEYMEAIYPVRSNST | SEQ ID NO:47 |
| PPEYMEAIYPVRSNSTI | SEQ ID NO:48 |
| PPEYMEAIYPVRSNSTIA | SEQ ID NO:49 |
| PPEYMEAIYPVRSNSTIAR | SEQ ID NO:50 |
| PPEYMEAIYPVRSNSTIARG | SEQ ID NO:51 |
| APPEYMEA | SEQ ID NO:52 |
| APPEYMEAI | SEQ ID NO:53 |
| APPEYMEAIY | SEQ ID NO:54 |
| APPEYMEAIYP | SEQ ID NO:55 |
| APPEYMEAIYPV | SEQ ID NO:56 |
| APPEYMEAIYPVR | SEQ ID NO:57 |
| APPEYMEAIYPVRS | SEQ ID NO:58 |
| APPEYMEAIYPVRSN | SEQ ID NO:59 |
| APPEYMEAIYPVRSNS | SEQ ID NO:60 |
| APPEYMEAIYPVRSNST | SEQ ID NO:61 |
| APPEYMEAIYPVRSNSTI | SEQ ID NO:62 |
| APPEYMEAIYPVRSNSTIA | SEQ ID NO:63 |
| APPEYMEAIYPVRSNSTIAR | SEQ ID NO:64 |
| TAPPEYME | SEQ ID NO:65 |
| TAPPEYMEA | SEQ ID NO:66 |
| TAPPEYMEAI | SEQ ID NO:67 |
| TAPPEYMEAIY | SEQ ID NO:68 |
| TAPPEYMEAIYP | SEQ ID NO:69 |
| TAPPEYMEAIYPV | SEQ ID NO:70 |
| TAPPEYMEAIYPVR | SEQ ID NO:71 |
| TAPPEYMEAIYPVRS | SEQ ID NO:72 |
| TAPPEYMEAIYPVRSN | SEQ ID NO:73 |
| TAPPEYMEAIYPVRSNS | SEQ ID NO:74 |
| TAPPEYMEAIYPVRSNST | SEQ ID NO:75 |
| TAPPEYMEAIYPVRSNSTI | SEQ ID NO:76 |
| TAPPEYMEAIYPVRSNSTIA | SEQ ID NO:77 |
| PTAPPEYM | SEQ ID NO:78 |
| PTAPPEYME | SEQ ID NO:79 |
| PTAPPEYMEA | SEQ ID NO:80 |
| PTAPPEYMEAI | SEQ ID NO:81 |
| PTAPPEYMEAIY | SEQ ID NO:82 |
| PTAPPEYMEAIYP | SEQ ID NO:83 |
| PTAPPEYMEAIYPV | SEQ ID NO:84 |
| PTAPPEYMEAIYPVR | SEQ ID NO:85 |
| PTAPPEYMEAIYPVRS | SEQ ID NO:86 |
| PTAPPEYMEAIYPVRSN | SEQ ID NO:87 |
| PTAPPEYMEAIYPVRSNS | SEQ ID NO:88 |
| PTAPPEYMEAIYPVRSNST | SEQ ID NO:89 |
| PTAPPEYMEAIYPVRSNSTI | SEQ ID NO:90 |
| LPTAPPEY | SEQ ID NO:91 |
| LPTAPPEYM | SEQ ID NO:92 |
| LPTAPPEYME | SEQ ID NO:93 |
| LPTAPPEYMEA | SEQ ID NO:94 |
| LPTAPPEYMEAI | SEQ ID NO:95 |
| LPTAPPEYMEAIY | SEQ ID NO:96 |
| LPTAPPEYMEAIYP | SEQ ID NO:97 |
| LPTAPPEYMEAIYPV | SEQ ID NO:98 |
| LPTAPPEYMEAIYPVR | SEQ ID NO:99 |
| LPTAPPEYMEAIYPVRS | SEQ ID NO:100 |
| LPTAPPEYMEAIYPVRSN | SEQ ID NO:101 |
| LPTAPPEYMEAIYPVRSNS | SEQ ID NO:102 |
| LPTAPPEYMEAIYPVRSNST | SEQ ID NO:103 |
| ILPTAPPEY | SEQ ID NO:104 |
| ILPTAPPEYM | SEQ ID NO:105 |
| ILPTAPPEYME | SEQ ID NO:106 |
| ILPTAPPEYMEA | SEQ ID NO:107 |
| ILPTAPPEYMEAI | SEQ ID NO:108 |
| ILPTAPPEYMEAIY | SEQ ID NO:109 |
| ILPTAPPEYMEAIYP | SEQ ID NO:110 |
| ILPTAPPEYMEAIYPV | SEQ ID NO:111 |
| ILPTAPPEYMEAIYPVR | SEQ ID NO:112 |
| ILPTAPPEYMEAIYPVRS | SEQ ID NO:113 |
| ILPTAPPEYMEAIYPVRSN | SEQ ID NO:114 |
| ILPTAPPEYMEAIYPVRSNS | SEQ ID NO:115 |
| VILPTAPPEY | SEQ ID NO:116 |
| VILPTAPPEYM | SEQ ID NO:117 |
| VILPTAPPEYME | SEQ ID NO:118 |
| VILPTAPPEYMEA | SEQ ID NO:119 |
| VILPTAPPEYMEAI | SEQ ID NO:120 |
| VILPTAPPEYMEAIY | SEQ ID NO:121 |
| VILPTAPPEYMEAIYP | SEQ ID NO:122 |
| VILPTAPPEYMEAIYPV | SEQ ID NO:123 |
| VILPTAPPEYMEAIYPVR | SEQ ID NO:124 |
| VILPTAPPEYMEAIYPVRS | SEQ ID NO:125 |
| VILPTAPPEYMEAIYPVRSN | SEQ ID NO:126 |
| RVILPTAPPEY | SEQ ID NO:127 |
| RVILPTAPPEYM | SEQ ID NO:128 |
| RVILPTAPPEYME | SEQ ID NO:129 |
| RVILPTAPPEYMEA | SEQ ID NO:130 |
| RVILPTAPPEYMEAI | SEQ ID NO:131 |
| RVILPTAPPEYMEAIY | SEQ ID NO:132 |
| RVILPTAPPEYMEAIYP | SEQ ID NO:133 |
| RVILPTAPPEYMEAIYPV | SEQ ID NO:134 |
| RVILPTAPPEYMEAIYPVR | SEQ ID NO:135 |
| RVILPTAPPEYMEAIYPVRS | SEQ ID NO:136 |
| RRVILPTAPPEY | SEQ ID NO:137 |
| RRVILPTAPPEYM | SEQ ID NO:138 |
| RRVILPTAPPEYME | SEQ ID NO:139 |
| RRVILPTAPPEYMEA | SEQ ID NO:140 |
| RRVILPTAPPEYMEAI | SEQ ID NO:141 |
| RRVILPTAPPEYMEAIY | SEQ ID NO:142 |
| RRVILPTAPPEYMEAIYP | SEQ ID NO:143 |
| RRVILPTAPPEYMEAIYPV | SEQ ID NO:144 |
| RRVILPTAPPEYMEAIYPVR | SEQ ID NO:145 |
| MRRVILPTAPPEY | SEQ ID NO:146 |
| MRRVILPTAPPEYM | SEQ ID NO:147 |
| MRRVILPTAPPEYME | SEQ ID NO:148 |
| MRRVILPTAPPEYMEA | SEQ ID NO:149 |
| MRRVILPTAPPEYMEAI | SEQ ID NO:150 |
| MRRVILPTAPPEYMEAIY | SEQ ID NO:151 |
| MRRVILPTAPPEYMEAIYP | SEQ ID NO:152 |
| MRRVILPTAPPEYMEAIYPV | SEQ ID NO:153 |

TABLE 6

PPXY Motif Containing Peptides from Hepatitis B
Virus Core Antigen
(GenBank Accession No. S53155)

| | |
|---|---|
| PPPYRPPN | SEQ ID NO:582 |
| PPPYRPPNA | SEQ ID NO:583 |
| PPPYRPPNAP | SEQ ID NO:584 |
| PPPYRPPNAPI | SEQ ID NO:585 |
| PPPYRPPNAPIL | SEQ ID NO:586 |
| PPPYRPPNAPILS | SEQ ID NO:587 |
| PPPYRPPNAPILST | SEQ ID NO:588 |
| PPPYRPPNAPILSTL | SEQ ID NO:589 |
| PPPYRPPNAPILSTLP | SEQ ID NO:590 |
| PPPYRPPNAPILSTLPE | SEQ ID NO:591 |
| PPPYRPPNAPILSTLPET | SEQ ID NO:592 |
| PPPYRPPNAPILSTLPETT | SEQ ID NO:593 |
| PPPYRPPNAPILSTLPETTV | SEQ ID NO:594 |
| TPPPYRPP | SEQ ID NO:595 |
| TPPPYRPPN | SEQ ID NO:596 |
| TPPPYRPPNA | SEQ ID NO:597 |
| TPPPYRPPNAP | SEQ ID NO:598 |
| TPPPYRPPNAPI | SEQ ID NO:599 |
| TPPPYRPPNAPIL | SEQ ID NO:600 |
| TPPPYRPPNAPILS | SEQ ID NO:601 |
| TPPPYRPPNAPILST | SEQ ID NO:602 |
| TPPPYRPPNAPILSTL | SEQ ID NO:603 |
| TPPPYRPPNAPILSTLP | SEQ ID NO:604 |
| TPPPYRPPNAPILSTLPE | SEQ ID NO:605 |
| TPPPYRPPNAPILSTLPET | SEQ ID NO:606 |
| TPPPYRPPNAPILSTLPETT | SEQ ID NO:607 |
| RTPPPYRP | SEQ ID NO:608 |
| RTPPPYRPP | SEQ ID NO:609 |
| RTPPPYRPPN | SEQ ID NO:610 |
| RTPPPYRPPNA | SEQ ID NO:611 |
| RTPPPYRPPNAP | SEQ ID NO:612 |
| RTPPPYRPPNAPI | SEQ ID NO:613 |
| RTPPPYRPPNAPIL | SEQ ID NO:614 |
| RTPPPYRPPNAPILS | SEQ ID NO:615 |
| RTPPPYRPPNAPILST | SEQ ID NO:616 |
| RTPPPYRPPNAPILSTL | SEQ ID NO:617 |
| RTPPPYRPPNAPILSTLP | SEQ ID NO:618 |
| RTPPPYRPPNAPILSTLPE | SEQ ID NO:619 |
| RTPPPYRPPNAPILSTLPET | SEQ ID NO:620 |
| IRTPPPYR | SEQ ID NO:621 |
| IRTPPPYRP | SEQ ID NO:622 |
| IRTPPPYRPP | SEQ ID NO:623 |
| IRTPPPYRPPN | SEQ ID NO:624 |
| IRTPPPYRPPNA | SEQ ID NO:625 |
| IRTPPPYRPPNAP | SEQ ID NO:626 |
| IRTPPPYRPPNAPI | SEQ ID NO:627 |
| IRTPPPYRPPNAPIL | SEQ ID NO:628 |
| IRTPPPYRPPNAPILS | SEQ ID NO:629 |
| IRTPPPYRPPNAPILST | SEQ ID NO:630 |
| IRTPPPYRPPNAPILSTL | SEQ ID NO:631 |
| IRTPPPYRPPNAPILSTLP | SEQ ID NO:632 |
| IRTPPPYRPPNAPILSTLPE | SEQ ID NO:633 |
| WIRTPPPY | SEQ ID NO:634 |
| WIRTPPPYR | SEQ ID NO:635 |
| WIRTPPPYRP | SEQ ID NO:636 |
| WIRTPPPYRPP | SEQ ID NO:637 |
| WIRTPPPYRPPN | SEQ ID NO:638 |
| WIRTPPPYRPPNA | SEQ ID NO:639 |
| WIRTPPPYRPPNAP | SEQ ID NO:640 |
| WIRTPPPYRPPNAPI | SEQ ID NO:641 |
| WIRTPPPYRPPNAPIL | SEQ ID NO:642 |
| WIRTPPPYRPPNAPILS | SEQ ID NO:643 |
| WIRTPPPYRPPNAPILST | SEQ ID NO:644 |
| WIRTPPPYRPPNAPILSTL | SEQ ID NO:645 |
| WIRTPPPYRPPNAPILSTLP | SEQ ID NO:646 |
| VWIRTPPPY | SEQ ID NO:647 |
| VWIRTPPPYR | SEQ ID NO:648 |
| VWIRTPPPYRP | SEQ ID NO:649 |
| VWIRTPPPYRPP | SEQ ID NO:650 |
| VWIRTPPPYRPPN | SEQ ID NO:651 |
| VWIRTPPPYRPPNA | SEQ ID NO:652 |
| VWIRTPPPYRPPNAP | SEQ ID NO:653 |
| VWIRTPPPYRPPNAPI | SEQ ID NO:654 |
| VWIRTPPPYRPPNAPIL | SEQ ID NO:655 |
| VWIRTPPPYRPPNAPILS | SEQ ID NO:656 |
| VWIRTPPPYRPPNAPILST | SEQ ID NO:657 |
| VWIRTPPPYRPPNAPILSTL | SEQ ID NO:658 |
| GVWIRTPPPY | SEQ ID NO:659 |
| GVWIRTPPPYR | SEQ ID NO:660 |
| GVWIRTPPPYRP | SEQ ID NO:661 |
| GVWTRTPPPYRPP | SEQ ID NO:662 |
| GVWIRTPPPYRPPN | SEQ ID NO:663 |
| GVWIRTPPPYRPPNA | SEQ ID NO:664 |
| GVWIRTPPPYRPPNAP | SEQ ID NO:665 |
| GVWIRTPPPYRPPNAPI | SEQ ID NO:666 |
| GVWIRTPPPYRPPNAPIL | SEQ ID NO:667 |
| GVWIRTPPPYRPPNAPILS | SEQ ID NO:668 |
| GVWIRTPPPYRPPNAPILST | SEQ ID NO:669 |
| FGVWIRTPPPY | SEQ ID NO:670 |
| FGVWIRTPPPYR | SEQ ID NO:671 |
| FGVWIRTPPPYRP | SEQ ID NO:672 |
| FGVWIRTPPPYRPP | SEQ ID NO:673 |
| FGVWIRTPPPYRPPN | SEQ ID NO:674 |
| FGVWIRTPPPYRPPNA | SEQ ID NO:675 |
| FGVWIRTPPPYRPPNAP | SEQ ID NO:676 |
| FGVWIRTPPPYRPPNAPI | SEQ ID NO:677 |
| FGVWIRTPPPYRPPNAPIL | SEQ ID NO:678 |
| FGVWIRTPPPYRPPNAPILS | SEQ ID NO:679 |
| SFGVWIRTPPPY | SEQ ID NO:680 |
| SFGVWIRTPPPYR | SEQ ID NO:681 |
| SFGVWIRTPPPYRP | SEQ ID NO:682 |
| SFGVWIRTPPPYRPP | SEQ ID NO:683 |
| SFGVWIRTPPPYRPPN | SEQ ID NO:684 |
| SFGVWIRTPPPYRPPNA | SEQ ID NO:685 |
| SFGVWIRTPPPYRPPNAP | SEQ ID NO:686 |
| SFGVWIRTPPPYRPPNAPI | SEQ ID NO:687 |
| SFGVWIRTPPPYRPPNAPIL | SEQ ID NO:688 |
| VSFGVWIRTPPPY | SEQ ID NO:689 |
| VSFGVWIRTPPPYR | SEQ ID NO:690 |
| VSFGVWIRTPPPYRP | SEQ ID NO:691 |
| VSFGVWIRTPPPYRPP | SEQ ID NO:692 |
| VSFGVWIRTPPPYRPPN | SEQ ID NO:693 |
| VSFGVWIRTPPPYRPPNA | SEQ ID NO:694 |
| VSFGVWIRTPPPYRPPNAP | SEQ ID NO:695 |
| VSFGVWIRTPPPYRPPNAPI | SEQ ID NO:696 |
| LVSFGVWIRTPPPY | SEQ ID NO:697 |
| LVSFGVWIRTPPPYR | SEQ ID NO:698 |
| LVSFGVWIRTPPPYRP | SEQ ID NO:699 |
| LVSFGVWIRTPPPYRPP | SEQ ID NO:700 |
| LVSFGVWIRTPPPYRPPN | SEQ ID NO:701 |
| LVSFGVWTRTPPPYRPPNA | SEQ ID NO:702 |
| LVSFGVWIRTPPPYRPPNAP | SEQ ID NO:703 |
| YLVSFGVWIRTPPPY | SEQ ID NO:704 |
| YLVSFGVWIRTPPPYR | SEQ ID NO:705 |
| YLVSFGVWIRTPPPYRP | SEQ ID NO:706 |
| YLVSFGVWIRTPPPYRPP | SEQ ID NO:707 |
| YLVSFGVWIRTPPPYRPPN | SEQ ID NO:708 |
| YLVSFGVWIRTPPPYRPPNA | SEQ ID NO:709 |
| EYLVSFGVWIRTPPPY | SEQ ID NO:710 |
| EYLVSFGVWIRTPPPYR | SEQ ID NO:711 |
| EYLVSFGVWIRTPPPYRP | SEQ ID NO:712 |
| EYLVSFGVWIRTPPPYRPP | SEQ ID NO:713 |
| EYLVSFGVWIRTPPPYRPPN | SEQ ID NO:714 |
| IEYLVSFGVWIRTPPPY | SEQ ID NO:715 |
| IEYLVSFGVWIRTPPPYR | SEQ ID NO:716 |
| IEYLVSFGVWIRTPPPYRP | SEQ ID NO:717 |
| IEYLVSFGVWIRTPPPYRPP | SEQ ID NO:718 |
| VIEYLVSFGVWIRTPPPY | SEQ ID NO:719 |
| VIEYLVSFGVWIRTPPPYR | SEQ ID NO:720 |
| VIEYLVSFGVWIRTPPPYRP | SEQ ID NO:721 |
| TVIEYLVSFGVWIRTPPPY | SEQ ID NO:722 |
| TVIEYLVSFGVWIRTPPPYR | SEQ ID NO:723 |
| DTVIEYLVSFGVWIRTPPPY | SEQ ID NO:724 |

TABLE 7

PPPY Motif Containing Peptides from Human
Herpesvirus 4 (Epstein-Barr Virus)
Latent Membrane Protein 2A
(GenBank Accession No. CAA57375)

| | |
|---|---|
| PPPYEDPY | SEQ ID NO:725 |
| PPPYEDPYW | SEQ ID NO:726 |
| PPPYEDPYWG | SEQ ID NO:727 |
| PPPYEDPYWGN | SEQ ID NO:728 |
| PPPYEDPYWGNG | SEQ ID NO:729 |
| PPPYEDPYWGNGD | SEQ ID NO:730 |
| PPPYEDPYWGNGDR | SEQ ID NO:731 |
| PPPYEDPYWGNGDRH | SEQ ID NO:732 |
| PPPYEDPYWGNGDRHS | SEQ ID NO:733 |
| PPPYEDPYWGNGDRHSD | SEQ ID NO:734 |
| PPPYEDPYWGNGDRHSDY | SEQ ID NO:735 |
| PPPYEDPYWGNGDRHSDYQ | SEQ ID NO:736 |
| PPPYEDPYWGNGDRHSDYQP | SEQ ID NO:737 |
| PPPPYEDP | SEQ ID NO:738 |
| PPPPYEDPY | SEQ ID NO:739 |
| PPPPYEDPYW | SEQ ID NO:740 |
| PPPPYEDPYWG | SEQ ID NO:741 |
| PPPPYEDPYWGN | SEQ ID NO:742 |
| PPPPYEDPYWGNG | SEQ ID NO:743 |
| PPPPYEDPYWGNGD | SEQ ID NO:744 |
| PPPPYEDPYWGNGDR | SEQ ID NO:745 |
| PPPPYEDPYWGNGDRH | SEQ ID NO:746 |
| PPPPYEDPYWGNGDRHS | SEQ ID NO:747 |
| PPPPYEDPYWGNGDRHSD | SEQ ID NO:748 |
| PPPPYEDPYWGNGDRHSDY | SEQ ID NO:749 |
| PPPPYEDPYWGNGDRHSDYQ | SEQ ID NO:750 |
| EPPPPYED | SEQ ID NO:751 |
| EPPPPYEDP | SEQ ID NO:752 |
| EPPPPYEDPY | SEQ ID NO:753 |
| EPPPPYEDPYW | SEQ ID NO:754 |
| EPPPPYEDPYWG | SEQ ID NO:755 |
| EPPPPYEDPYWGN | SEQ ID NO:756 |
| EPPPPYEDPYWGNG | SEQ ID NO:757 |
| EPPPPYEDPYWGNGD | SEQ ID NO:758 |
| EPPPPYEDPYWGNGDR | SEQ ID NO:759 |
| EPPPPYEDPYWGNGDRH | SEQ ID NO:760 |
| EPPPPYEDPYWGNGDRHS | SEQ ID NO:761 |
| EPPPPYEDPYWGNGDRHSD | SEQ ID NO:762 |
| EPPPPYEDPYWGNGDRHSDY | SEQ ID NO:763 |
| EEPPPPYE | SEQ ID NO:764 |
| EEPPPPYED | SEQ ID NO:765 |
| EEPPPPYEDP | SEQ ID NO:766 |
| EEPPPPYEDPY | SEQ ID NO:767 |
| EEPPPPYEDPYW | SEQ ID NO:768 |
| EEPPPPYEDPYWG | SEQ ID NO:769 |
| EEPPPPYEDPYWGN | SEQ ID NO:770 |
| EEPPPPYEDPYWGNG | SEQ ID NO:771 |
| EEPPPPYEDPYWGNGD | SEQ ID NO:772 |
| EEPPPPYEDPYWGNGDR | SEQ ID NO:773 |
| EEPPPPYEDPYWGNGDRH | SEQ ID NO:774 |
| EEPPPPYEDPYWGNGDRHS | SEQ ID NO:775 |
| EEPPPPYEDPYWGNGDRHSD | SEQ ID NO:776 |
| NEEPPPPY | SEQ ID NO:777 |
| NEEPPPPYE | SEQ ID NO:778 |
| NEEPPPPYED | SEQ ID NO:779 |
| NEEPPPPYEDP | SEQ ID NO:780 |
| NEEPPPPYEDPY | SEQ ID NO:781 |
| NEEPPPPYEDPYW | SEQ ID NO:782 |
| NEEPPPPYEDPYWG | SEQ ID NO:783 |
| NEEPPPPYEDPYWGN | SEQ ID NO:784 |
| NEEPPPPYEDPYWGNG | SEQ ID NO:785 |
| NEEPPPPYEDPYWGNGD | SEQ ID NO:786 |
| NEEPPPPYEDPYWGNGDR | SEQ ID NO:787 |
| NEEPPPPYEDPYWGNGDRH | SEQ ID NO:788 |
| NEEPPPPYEDPYWGNGDRHS | SEQ ID NO:789 |
| SNEEPPPPY | SEQ ID NO:790 |
| SNEEPPPPYE | SEQ ID NO:791 |
| SNEEPPPPYED | SEQ ID NO:792 |
| SNEEPPPPYEDP | SEQ ID NO:793 |
| SNEEPPPPYEDPY | SEQ ID NO:794 |
| SNEEPPPPYEDPYW | SEQ ID NO:795 |
| SNEEPPPPYEDPYWG | SEQ ID NO:796 |
| SNEEPPPPYEDPYWGN | SEQ ID NO:797 |
| SNEEPPPPYEDPYWGNG | SEQ ID NO:798 |
| SNEEPPPPYEDPYWGNGD | SEQ ID NO:799 |
| SNEEPPPPYEDPYWGNGDR | SEQ ID NO:800 |
| SNEEPPPPYEDPYWGNGDRH | SEQ ID NO:801 |
| ESNEEPPPPY | SEQ ID NO:802 |
| ESNEEPPPPYE | SEQ ID NO:803 |
| ESNEEPPPPYED | SEQ ID NO:804 |
| ESNEEPPPPYEDP | SEQ ID NO:805 |
| ESNEEPPPPYEDPY | SEQ ID NO:806 |
| ESNEEPPPPYEDPYW | SEQ ID NO:807 |
| ESNEEPPPPYEDPYWG | SEQ ID NO:808 |
| ESNEEPPPPYEDPYWGN | SEQ ID NO:809 |
| ESNEEPPPPYEDPYWGNG | SEQ ID NO:810 |
| ESNEEPPPPYEDPYWGNGD | SEQ ID NO:811 |
| SNEEPPPPYEDPYWGNGDR | SEQ ID NO:812 |
| RESNEEPPPPY | SEQ ID NO:813 |
| RESNEEPPPPYE | SEQ ID NO:814 |
| RESNEEPPPPYED | SEQ ID NO:815 |
| RESNEEPPPPYEDP | SEQ ID NO:816 |
| RESNEEPPPPYEDPY | SEQ ID NO:817 |
| RESNEEPPPPYEDPYW | SEQ ID NO:818 |
| RESNEEPPPPYEDPYWG | SEQ ID NO:819 |
| RESNEEPPPPYEDPYWGN | SEQ ID NO:820 |
| RESNEEPPPPYEDPYWGNG | SEQ ID NO:821 |
| RESNEEPPPPYEDPYWGNGD | SEQ ID NO:822 |
| ERESNEEPPPPY | SEQ ID NO:823 |
| ERESNEEPPPPYE | SEQ ID NO:824 |
| ERESNEEPPPPYED | SEQ ID NO:825 |
| ERESNEEPPPPYEDP | SEQ ID NO:826 |
| ERESNEEPPPPYEDPY | SEQ ID NO:827 |
| ERESNEEPPPPYEDPYW | SEQ ID NO:828 |
| ERESNEEPPPPYEDPYWG | SEQ ID NO:829 |
| ERESNEEPPPPYEDPYWGN | SEQ ID NO:830 |
| ERESNEEPPPPYEDPYWGNG | SEQ ID NO:831 |
| EERESNEEPPPPY | SEQ ID NO:832 |
| EERESNEEPPPPYE | SEQ ID NO:833 |
| EERESNEEPPPPYED | SEQ ID NO:834 |
| EERESNEEPPPPYEDP | SEQ ID NO:835 |
| EERESNEEPPPPYEDPY | SEQ ID NO:836 |
| EERESNEEPPPPYEDPYW | SEQ ID NO:837 |
| EERESNEEPPPPYEDPYWG | SEQ ID NO:838 |
| EERESNEEPPPPYEDPYWGN | SEQ ID NO:839 |
| DEERESNEEPPPPY | SEQ ID NO:840 |
| DEERESNEEPPPPYE | SEQ ID NO:841 |
| DEERESNEEPPPPYED | SEQ ID NO:842 |
| DEERESNEEPPPPYEDP | SEQ ID NO:843 |
| DEERESNEEPPPPYEDPY | SEQ ID NO:844 |
| DEERESNEEPPPPYEDPYW | SEQ ID NO:845 |
| DEERESNEEPPPPYEDPYWG | SEQ ID NO:846 |
| NDEERESNEEPPPPY | SEQ ID NO:847 |
| NDEERESNEEPPPPYE | SEQ ID NO:848 |
| NDEERESNEEPPPPYED | SEQ ID NO:849 |
| NDEERESNEEPPPPYEDP | SEQ ID NO:850 |
| NDEERESNEEPPPPYEDPY | SEQ ID NO:851 |
| NDEERESNEEPPPPYEDPYW | SEQ ID NO:852 |
| PNDEERESNEEPPPPY | SEQ ID NO:853 |
| PNDEERESNEEPPPPYE | SEQ ID NO:854 |
| PNDEERESNEEPPPPYED | SEQ ID NO:855 |
| PNDEERESNEEPPPPYEDP | SEQ ID NO:856 |
| PNDEERESNEEPPPPYEDPY | SEQ ID NO:857 |
| PPNDEERESNEEPPPPY | SEQ ID NO:858 |
| PPNDEERESNEEPPPPYE | SEQ ID NO:859 |
| PPNDEERESNEEPPPPYED | SEQ ID NO:860 |
| PPNDEERESNEEPPPPYEDP | SEQ ID NO:861 |
| TPPNDEERESNEEPPPPY | SEQ ID NO:862 |
| TPPNDEERESNEEPPPPYE | SEQ ID NO:863 |
| TPPNDEERESNEEPPPPYED | SEQ ID NO:864 |
| PTPPNDEERESNEEPPPPY | SEQ ID NO:865 |
| PTPPNDEERESNEEPPPPYE | SEQ ID NO:866 |
| TPTPPNDEERESNEEPPPPY | SEQ ID NO:867 |
| PPPYSPRD | SEQ ID NO:868 |
| PPPYSPRDD | SEQ ID NO:869 |
| PPPYSPRDDS | SEQ ID NO:870 |
| PPPYSPRDDSS | SEQ ID NO:871 |
| PPPYSPRDDSSQ | SEQ ID NO:872 |
| PPPYSPRDDSSQH | SEQ ID NO:873 |
| PPPYSPRDDSSQHI | SEQ ID NO:874 |
| PPPYSPRDDSSQHIY | SEQ ID NO:875 |
| PPPYSPRDDSSQHIYE | SEQ ID NO:876 |
| PPPYSPRDDSSQHIYEE | SEQ ID NO:877 |
| PPPYSPRDDSSQHIYEEA | SEQ ID NO:878 |
| PPPYSPRDDSSQHIYEEAD | SEQ ID NO:879 |
| PPPYSPRDDSSQHIYEEADR | SEQ ID NO:880 |
| PPPPYSPR | SEQ ID NO:881 |
| PPPPYSPRD | SEQ ID NO:882 |
| PPPPYSPRDD | SEQ ID NO:883 |
| PPPPYSPRDDS | SEQ ID NO:884 |
| PPPPYSPRDDSS | SEQ ID NO:885 |
| PPPPYSPRDDSSQ | SEQ ID NO:886 |
| PPPPYSPRDDSSQH | SEQ ID NO:887 |
| PPPPYSPRDDSSQHI | SEQ ID NO:888 |
| PPPPYSPRDDSSQHIY | SEQ ID NO:889 |
| PPPPYSPRDDSSQHIYE | SEQ ID NO:890 |
| PPPPYSPRDDSSQHIYEE | SEQ ID NO:891 |
| PPPPYSPRDDSSQHIYEEA | SEQ ID NO:892 |
| PPPPYSPRDDSSQHIYEEAD | SEQ ID NO:893 |
| LPPPPYSP | SEQ ID NO:894 |
| LPPPPYSPR | SEQ ID NO:895 |
| LPPPPYSPRD | SEQ ID NO:896 |
| LPPPPYSPRDD | SEQ ID NO:897 |
| LPPPPYSPRDDS | SEQ ID NO:898 |
| LPPPPYSPRDDSS | SEQ ID NO:899 |
| LPPPPYSPRDDSSQ | SEQ ID NO:900 |
| LPPPPYSPRDDSSQH | SEQ ID NO:901 |
| LPPPPYSPRDDSSQHI | SEQ ID NO:902 |
| LPPPPYSPRDDSSQHIY | SEQ ID NO:903 |
| LPPPPYSPRDDSSQHIYE | SEQ ID NO:904 |
| LPPPPYSPRDDSSQHIYEE | SEQ ID NO:905 |
| LPPPPYSPRDDSSQHIYEEA | SEQ ID NO:906 |
| GLPPPPYS | SEQ ID NO:907 |
| GLPPPPYSP | SEQ ID NO:908 |
| GLPPPPYSPR | SEQ ID NO:909 |
| GLPPPPYSPRD | SEQ ID NO:910 |
| GLPPPPYSPRDD | SEQ ID NO:911 |
| GLPPPPYSPRDDS | SEQ ID NO:912 |
| GLPPPPYSPRDDSS | SEQ ID NO:913 |
| GLPPPPYSPRDDSSQ | SEQ ID NO:914 |
| GLPPPPYSPRDDSSQH | SEQ ID NO:915 |
| GLPPPPYSPRDDSSQHI | SEQ ID NO:916 |
| LPPPPYSPRDDSSQHIY | SEQ ID NO:917 |
| GLPPPPYSPRDDSSQHIYE | SEQ ID NO:918 |
| GLPPPPYSPRDDSSQHIYEE | SEQ ID NO:919 |
| DGLPPPPY | SEQ ID NO:920 |
| DGLPPPPYS | SEQ ID NO:921 |
| DGLPPPPYSP | SEQ ID NO:922 |
| DGLPPPPYSPR | SEQ ID NO:923 |
| DGLPPPPYSPRD | SEQ ID NO:924 |
| DGLPPPPYSPRDD | SEQ ID NO:925 |
| DGLPPPPYSPRDDS | SEQ ID NO:926 |
| DGLPPPPYSPRDDSS | SEQ ID NO:927 |
| DGLPPPPYSPRDDSSQ | SEQ ID NO:928 |
| DGLPPPPYSPRDDSSQH | SEQ ID NO:929 |
| DGLPPPPYSPRDDSSQHI | SEQ ID NO:930 |
| DGLPPPPYSPRDDSSQHIY | SEQ ID NO:931 |
| DGLPPPPYSPRDDSSQHIYE | SEQ ID NO:932 |
| NDGLPPPPY | SEQ ID NO:933 |
| NDGLPPPPYS | SEQ ID NO:934 |
| NDGLPPPPYSP | SEQ ID NO:935 |
| NDGLPPPPYSPR | SEQ ID NO:936 |
| NDGLPPPPYSPRD | SEQ ID NO:937 |
| NDGLPPPPYSPRDD | SEQ ID NO:938 |
| NDGLPPPPYSPRDDS | SEQ ID NO:939 |
| NDGLPPPPYSPRDDSS | SEQ ID NO:940 |
| NDGLPPPPYSPRDDSSQ | SEQ ID NO:941 |
| NDGLPPPPYSPRDDSSQH | SEQ ID NO:942 |
| NDGLPPPPYSPRDDSSQHI | SEQ ID NO:943 |
| NDGLPPPPYSPRDDSSQHIY | SEQ ID NO:944 |
| GNDGLPPPPY | SEQ ID NO:945 |
| GNDGLPPPPYS | SEQ ID NO:946 |
| GNDGLPPPPYSP | SEQ ID NO:947 |
| GNDGLPPPPYSPR | SEQ ID NO:948 |
| GNDGLPPPPYSPRD | SEQ ID NO:949 |
| GNDGLPPPPYSPRDD | SEQ ID NO:950 |
| GNDGLPPPPYSPRDDS | SEQ ID NO:951 |
| GNDGLPPPPYSPRDDSS | SEQ ID NO:952 |
| GNDGLPPPPYSPRDDSSQ | SEQ ID NO:953 |
| GNDGLPPPPYSPRDDSSQH | SEQ ID NO:954 |
| GNPGLPPPPYSPRDDSSQHI | SEQ ID NO:955 |
| DGNDGLPPPPY | SEQ ID NO:956 |
| DGNDGLPPPPYS | SEQ ID NO:957 |
| DGNDGLPPPPYSP | SEQ ID NO:958 |
| DGNDGLPPPPYSPR | SEQ ID NO:959 |
| DGNDGLPPPPYSPRD | SEQ ID NO:960 |
| DGNDGLPPPPYSPRDD | SEQ ID NO:961 |
| DGNDGLPPPPYSPRDDS | SEQ ID NO:962 |
| DGNDGLPPPPYSPRDDSS | SEQ ID NO:963 |
| DGNDGLPPPPYSPRDDSSQ | SEQ ID NO:964 |
| DGNDGLPPPPYSPRDDSSQH | SEQ ID NO:965 |
| HDGNDGLPPPPY | SEQ ID NO:966 |
| HDGNDGLPPPPYS | SEQ ID NO:967 |
| HDGNDGLPPPPYSP | SEQ ID NO:968 |
| HDGNDGLPPPPYSPR | SEQ ID NO:969 |
| HDGNDGLPPPPYSPRD | SEQ ID NO:970 |
| HDGNDGLPPPPYSPRDD | SEQ ID NO:971 |
| HDGNDGLPPPPYSPRDDS | SEQ ID NO:972 |
| HDGNDGLPPPPYSPRDDSS | SEQ ID NO:973 |
| HDGNDGLPPPPYSPRDDSSQ | SEQ ID NO:974 |
| QHDGNDGLPPPPY | SEQ ID NO:975 |
| QHDGNDGLPPPPYS | SEQ ID NO:976 |
| QHDGNDGLPPPPYSP | SEQ ID NO:977 |
| QHDGNDGLPPPPYSPR | SEQ ID NO:978 |
| QHDGNDGLPPPPYSPRD | SEQ ID NO:979 |
| QHDGNDGLPPPPYSPRDD | SEQ ID NO:980 |
| QHDGNDGLPPPPYSPRDDS | SEQ ID NO:981 |
| QHDGNDGLPPPPYSPRDDSS | SEQ ID NO:982 |
| LQHDGNDGLPPPPY | SEQ ID NO:983 |
| LQHDGNDGLPPPPYS | SEQ ID NO:984 |
| LQHDGNDGLPPPPYSP | SEQ ID NO:985 |
| LQHDGNDGLPPPPYSPR | SEQ ID NO:986 |
| LQHDGNDGLPPPPYSPRD | SEQ ID NO:987 |
| LQHDGNDGLPPPPYSPRDD | SEQ ID NO:988 |
| LQHIDGNDGLPPPPYSPRDDS | SEQ ID NO:989 |
| GLQHDGNPGLPPPPY | SEQ ID NO:990 |
| GLQHDGNDGLPPPPYS | SEQ ID NO:991 |
| GLQHDGNDGLPPPPYSP | SEQ ID NO:992 |
| GLQHDGNDGLPPPPYSPR | SEQ ID NO:993 |
| GLQHDGNDGLPPPPYSPRD | SEQ ID NO:994 |
| GLQHDGNDGLPPPPYSPRDD | SEQ ID NO:995 |
| LGLQHDGNDGLPPPPY | SEQ ID NO:996 |
| LGLQHDGNDGLPPPPYS | SEQ ID NO:997 |
| LGLQHDGNDGLPPPPYSP | SEQ ID NO:998 |
| LGLQHDGNDGLPPPPYSPR | SEQ ID NO:999 |
| LGLQHDGNDGLPPPPYSPRD | SEQ ID NO:1000 |
| YLGLQHDGNDGLPPPPY | SEQ ID NO:1001 |
| YLGLQHDGNDGLPPPPYS | SEQ ID NO:1002 |
| YLGLQHDGNDGLPPPPYSP | SEQ ID NO:1003 |
| YLGLQHDGNDGLPPPPYSPR | SEQ ID NO:1004 |
| LYLGLQHDGNDGLPPPPY | SEQ ID NO:1005 |
| LYLGLQHDGNDGLPPPPYS | SEQ ID NO:1006 |
| LYLGLQHDGNDGLPPPPYSP | SEQ ID NO:1007 |
| SLYLGLQHDGNDGLPPPPY | SEQ ID NO:1008 |
| SLYLGLQHDGNDGLPPPPYS | SEQ ID NO:1009 |
| PSLYLGLQHDGNDGLPPPPY | SEQ ID NO:1010 |

TABLE 8

PPXY Motif Containing Peptides from Human
Herpesvirus 1 (Strain F) UL56 Protein
(GenBank Accession No. A43965)

| | |
|---|---|
| PPPYDSLS | SEQ ID NO:1011 |
| PPPYDSLSG | SEQ ID NO:1012 |
| PPPYDSLSGR | SEQ ID NO:1013 |
| PPPYDSLSGRN | SEQ ID NO:1014 |
| PPPYDSLSGRNE | SEQ ID NO:1015 |
| PPPYDSLSGRNEG | SEQ ID NO:1016 |
| PPPYDSLSGRNEGP | SEQ ID NO:1017 |
| PPPYDSLSGRNEGPF | SEQ ID NO:1018 |
| PPPYDSLSGRNEGPFV | SEQ ID NO:1019 |
| PPPYDSLSGRNEGPFVV | SEQ ID NO:1020 |
| PPPYDSLSGRNEGPFVVI | SEQ ID NO:1021 |
| PPPYDSLSGRNEGPFVVID | SEQ ID NO:1022 |
| PPPYDSLSGRNEGPFVVIDL | SEQ ID NO:1023 |
| PPPPYDSL | SEQ ID NO:1O24 |
| PPPPYDSLS | SEQ ID NO:1025 |
| PPPPYDSLSG | SEQ ID NO:1026 |
| PPPPYDSLSGR | SEQ ID NO:1027 |
| PPPPYDSLSGRN | SEQ ID NO:1028 |
| PPPPYDSLSGRNE | SEQ ID NO:1029 |
| PPPPYDSLSGRNEG | SEQ ID NO:1030 |
| PPPPYDSLSGRNEGP | SEQ ID NO:1031 |
| PPPPYDSLSGRNEGPF | SEQ ID NO:1032 |
| PPPPYDSLSGRNEGPFV | SEQ ID NO:1033 |
| PPPPYDSLSGRNEGPFVV | SEQ ID NO:1034 |
| PPPPYDSLSGRNEGPFVVI | SEQ ID NO:1035 |
| PPPPYDSLSGRNEGPFVVID | SEQ ID NO:1036 |
| DPPPPYDS | SEQ ID NO:1037 |
| DPPPPYDSL | SEQ ID NO:1038 |
| DPPPPYDSLS | SEQ ID NO:1039 |
| DPPPPYDSLSG | SEQ ID NO:1040 |
| DPPPPYDSLSGR | SEQ ID NO:1041 |
| DPPPPYDSLSGRN | SEQ ID NO:1042 |
| DPPPPYDSLSGRNE | SEQ ID NO:1043 |
| DPPPPYDSLSGRNEG | SEQ ID NO:1044 |
| DPPPPYDSLSGRNEGP | SEQ ID NO:1045 |
| DPPPPYDSLSGRNEGPF | SEQ ID NO:1046 |
| DPPPPYDSLSGRNEGPFV | SEQ ID NO:1047 |
| DPPPPYDSLSGRNEGPFVV | SEQ ID NO:1048 |
| DPPPPYDSLSGRNEGPFVVI | SEQ ID NO:1049 |
| ADPPPPYD | SEQ ID NO:1050 |
| ADPPPPYDS | SEQ ID NO:1051 |
| ADPPPPYDSL | SEQ ID NO:1052 |
| ADPPPPYDSLS | SEQ ID NO:1053 |
| ADPPPPYDSLSG | SEQ ID NO:1054 |
| ADPPPPYDSLSGR | SEQ ID NO:1055 |
| ADPPPPYDSLSGRN | SEQ ID NO:1056 |
| ADPPPPYDSLSGRNE | SEQ ID NO:1057 |
| ADPPPPYDSLSGRNEG | SEQ ID NO:1058 |
| ADPPPPYDSLSGRNEGP | SEQ ID NO:1059 |
| ADPPPPYDSLSGRNEGPF | SEQ ID NO:1060 |
| ADPPPPYDSLSGRNEGPFV | SEQ ID NO:1061 |
| ADPPPPYDSLSGRNEGPFVV | SEQ ID NO:1062 |
| FADPPPPY | SEQ ID NO:1063 |
| FADPPPPYD | SEQ ID NO:1064 |
| FADPPPPYDS | SEQ ID NO:1065 |
| FADPPPPYDSL | SEQ ID NO:1066 |
| FADPPPPYDSLS | SEQ ID NO:1067 |
| FADPPPPYDSLSG | SEQ ID NO:1068 |
| FADPPPPYDSLSGR | SEQ ID NO:1069 |
| FADPPPPYDSLSGRN | SEQ ID NO:1070 |
| FADPPPPYDSLSGRNE | SEQ ID NO:1071 |
| FADPPPPYDSLSGRNEG | SEQ ID NO:1072 |
| FADPPPPYDSLSGRNEGP | SEQ ID NO:1073 |
| FADPPPPYDSLSGRNEGPF | SEQ ID NO:1074 |
| FADPPPPYDSLSGRNEGPFV | SEQ ID NO:1075 |
| AFADPPPPY | SEQ ID NO:1076 |
| AFADPPPPYD | SEQ ID NO:1077 |
| AFADPPPPYDS | SEQ ID NO:1078 |
| AFADPPPPYDSL | SEQ ID NO:1079 |
| AFADPPPPYDSLS | SEQ ID NO:1080 |
| AFADPPPPYDSLSG | SEQ ID NO:1081 |
| AFADPPPPYDSLSGR | SEQ ID NO:1082 |
| AFADPPPPYDSLSGRN | SEQ ID NO:1083 |
| AFADPPPPYDSLSGRNE | SEQ ID NO:1084 |
| AFADPPPPYDSLSGRNEG | SEQ ID NO:1085 |
| AFADPPPPYDSLSGRNEGP | SEQ ID NO:1086 |
| AFADPPPPYDSLSGRNEGPF | SEQ ID NO:1087 |
| NAFADPPPPY | SEQ ID NO:1088 |
| NAFADPPPPYD | SEQ ID NO:1089 |
| NAFADPPPPYDS | SEQ ID NO:1090 |
| NAFADPPPPYDSL | SEQ ID NO:1091 |
| NAFADPPPPYDSLS | SEQ ID NO:1092 |
| NAFADPPPPYDSLSG | SEQ ID NO:1093 |
| NAFADPPPPYDSLSGR | SEQ ID NO:1094 |
| NAFADPPPPYDSLSGRN | SEQ ID NO:1095 |
| NAFADPPPPYDSLSGRNE | SEQ ID NO:1096 |
| NAFADPPPPYDSLSGRNEG | SEQ ID NO:1097 |
| NAFADPPPPYDSLSGRNEGP | SEQ ID NO:1098 |
| GNAFADPPPPY | SEQ ID NO:1099 |
| GNAFADPPPPYD | SEQ ID NO:1100 |
| GNAFADPPPPYDS | SEQ ID NO:1101 |
| GNAFADPPPPYDSL | SEQ ID NO:1102 |
| GNAFADPPPPYDSLS | SEQ ID NO:1103 |
| GNAFADPPPPYDSLSG | SEQ ID NO:1104 |
| GNAFADPPPPYDSLSGR | SEQ ID NO:1105 |
| GNAFADPPPPYDSLSGRN | SEQ ID NO:1106 |
| GNAFADPPPPYDSLSGRNE | SEQ ID NO:1107 |
| GNAFADPPPPYDSLSGRNEG | SEQ ID NO:1108 |
| AGNAFADPPPPY | SEQ ID NO:1109 |
| AGNAFADPPPPYD | SEQ ID NO:1110 |
| AGNAFADPPPPYDS | SEQ ID NO:1111 |
| AGNAFADPPPPYDSL | SEQ ID NO:1112 |
| AGNAFADPPPPYDSLS | SEQ ID NO:1113 |
| AGNAFADPPPPYDSLSG | SEQ ID NO:1114 |
| AGNAFADPPPPYDSLSGR | SEQ ID NO:1115 |
| AGNAFADPPPPYDSLSGRN | SEQ ID NO:1116 |
| AGNAFADPPPPYDSLSGRNE | SEQ ID NO:1117 |
| SAGNAFADPPPPY | SEQ ID NO:1118 |
| SAGNAFADPPPPYD | SEQ ID NO:1119 |
| SAGNAFADPPPPYDS | SEQ ID NO:1120 |
| SAGNAFADPPPPYDSL | SEQ ID NO:1121 |
| SAGNAFADPPPPYDSLS | SEQ ID NO:1122 |
| SAGNAFADPPPPYDSLSG | SEQ ID NO:1123 |
| SAGNAFADPPPPYDSLSGR | SEQ ID NO:1124 |
| SAGNAFADPPPPYDSLSGRN | SEQ ID NO:1125 |
| WSAGNAFADPPPPY | SEQ ID NO:1126 |
| WSAGNAFADPPPPYD | SEQ ID NO:1127 |
| WSAGNAFADPPPPYDS | SEQ ID NO:1128 |
| WSAGNAFADPPPPYDSL | SEQ ID NO:1129 |
| WSAGNAFADPPPPYDSLS | SEQ ID NO:1130 |
| WSAGNAFADPPPPYDSLSG | SEQ ID NO:1131 |
| WSAGNAFADPPPPYDSLSGR | SEQ ID NO:1132 |
| LWSAGNAFADPPPPY | SEQ ID NO:1133 |
| LWSAGNAFADPPPPYD | SEQ ID NO:1134 |
| LWSAGNAFADPPPPYDS | SEQ ID NO:1135 |
| LWSAGNAFADPPPPYDSL | SEQ ID NO:1136 |
| LWSAGNAFADPPPPYDSLS | SEQ ID NO:1137 |
| LWSAGNAFADPPPPYDSLSG | SEQ ID NO:1138 |
| GLWSAGNAFADPPPPY | SEQ ID NO:1139 |
| GLWSAGNAFADPPPPYD | SEQ ID NO:1140 |
| GLWSAGNAFADPPPPYDS | SEQ ID NO:1141 |
| GLWSAGNAFADPPPPYDSL | SEQ ID NO:1142 |
| GLWSAGNAFADPPPPYDSLS | SEQ ID NO:1143 |
| AGLWSAGNAFADPPPPY | SEQ ID NO:1144 |
| AGLWSAGNAFADPPPPYD | SEQ ID NO:1145 |
| AGLWSAGNAFADPPPPYDS | SEQ ID NO:1146 |
| AGLWSAGNAFADPPPPYDSL | SEQ ID NO:1147 |
| DAGLWSAGNAFADPPPPY | SEQ ID NO:1148 |
| DAGLWSAGNAFADPPPPYD | SEQ ID NO:1149 |
| DAGLWSAGNAFADPPPPYDS | SEQ ID NO:1150 |
| PDAGLWSAGNAPADPPPPY | SEQ ID NO:1151 |
| PDAGLWSAGNAFADPPPPYD | SEQ ID NO:1152 |
| QPDAGLWSAGNAFADPPPPY | SEQ ID NO:1153 |
| PPPYSAGP | SEQ ID NO:1154 |
| PPPYSAGPL | SEQ ID NO:1155 |
| PPPYSAGPLL | SEQ ID NO:1156 |
| PPPYSAGPLLS | SEQ ID NO:1157 |
| PPPYSAGPLLSV | SEQ ID NO:1158 |
| PPPYSAGPLLSVP | SEQ ID NO:1159 |
| PPPYSAGPLLSVPI | SEQ ID NO:1160 |
| PPPYSAGPLLSVPIP | SEQ ID NO:1161 |
| PPPYSAGPLLSVPIPP | SEQ ID NO:1162 |
| PPPYSAGPLLSVPIPPT | SEQ ID NO:1163 |
| PPPYSAGPLLSVPIPPTS | SEQ ID NO:1164 |
| PPPYSAGPLLSVPIPPTSS | SEQ ID NO:1165 |
| PPPYSAGPLLSVPIIPPTSSG | SEQ ID NO:1166 |
| PPPPYSAG | SEQ ID NO:1167 |
| PPPPYSAGP | SEQ ID NO:1168 |
| PPPPYSAGPL | SEQ ID NO:1169 |
| PPPPYSAGPLL | SEQ ID NO:1170 |
| PPPPYSAGPLLS | SEQ ID NO:1171 |
| PPPPYSAGPLLSV | SEQ ID NO:1172 |
| PPPPYSAGPLLSVP | SEQ ID NO:1173 |
| PPPPYSAGPLLSVPI | SEQ ID NO:1174 |
| PPPPYSAGPLLSVPIP | SEQ ID NO:1175 |
| PPPPYSAGPLLSVPIPP | SEQ ID NO:1176 |
| PPPPYSAGPLLSVPIPPT | SEQ ID NO:1177 |
| PPPPYSAGPLLSVPIPPTS | SEQ ID NO:1178 |
| PPPPYSAGPLLSVPIPPTSS | SEQ ID NO:1179 |
| DPPPPYSA | SEQ ID NO:1180 |
| DPPPPYSAG | SEQ ID NO:1181 |
| DPPPPYSAGP | SEQ ID NO:1182 |
| DPPPPYSAGPL | SEQ ID NO:1183 |
| DPPPPYSAGPLL | SEQ ID NO:1184 |
| DPPPPYSAGPLLS | SEQ ID NO:1185 |
| DPPPPYSAGPLLSV | SEQ ID NO:1186 |
| DPPPPYSAGPLLSVP | SEQ ID NO:1187 |
| DPPPPYSAGPLLSVPI | SEQ ID NO:1188 |
| DPPPPYSAGPLLSVPIP | SEQ ID NO:1189 |
| DPPPPYSAGPLLSVPIPP | SEQ ID NO:1190 |
| DPPPPYSAGPLLSVPIPPT | SEQ ID NO:1191 |
| DPPPPYSAGPLLSVPIPPTS | SEQ ID NO:1192 |
| TDPPPPYS | SEQ ID NO:1193 |
| TDPPPPYSA | SEQ ID NO:1194 |
| TDPPPPYSAG | SEQ ID NO:1195 |
| TDPPPPYSAGP | SEQ ID NO:1196 |
| TDPPPPYSAGPL | SEQ ID NO:1197 |
| TDPPPPYSAGPLL | SEQ ID NO:1198 |
| TDPPPPYSAGPLLS | SEQ ID NO:1199 |
| TDPPPPYSAGPLLSV | SEQ ID NO:1200 |
| TDPPPPYSAGPLLSVP | SEQ ID NO:1201 |
| TDPPPPYSAGPLLSVPI | SEQ ID NO:1202 |
| TDPPPPYSAGPLLSVPIP | SEQ ID NO:1203 |
| TDPPPPYSAGPLLSVPIPP | SEQ ID NO:1204 |
| TDPPPPYSAGPLLSVPIIPPT | SEQ ID NO:1205 |
| PTDPPPPY | SEQ ID NO:1206 |
| PTDPPPPYS | SEQ ID NO:1207 |
| PTDPPPPYSA | SEQ ID NO:1208 |
| PTDPPPPYSAG | SEQ ID NO:1209 |
| PTDPPPPYSAGP | SEQ ID NO:1210 |
| PTDPPPPYSAGPL | SEQ ID NO:1211 |
| PTDPPPPYSAGPLL | SEQ ID NO:1212 |
| PTDPPPPYSAGPLLS | SEQ ID NO:1213 |
| PTDPPPPYSAGPLLSV | SEQ ID NO:1214 |
| PTDPPPPYSAGPLLSVP | SEQ ID NO:1215 |
| PTDPPPPYSAGPLLSVPI | SEQ ID NO:1216 |
| PTDPPPPYSAGPLLSVPIP | SEQ ID NO:1217 |
| PTDPPPPYSAGPLLSVPTPP | SEQ ID NO:1218 |
| TPTDPPPPY | SEQ ID NO:1219 |
| TPTDPPPPYS | SEQ ID NO:1220 |
| TPTDPPPPYSA | SEQ ID NO:1221 |
| TPTDPPPPYSAG | SEQ ID NO:1222 |
| TPTDPPPPYSAGP | SEQ ID NO:1223 |
| TPTDPPPPYSAGPL | SEQ ID NO:1224 |
| TPTDPPPPYSAGPLL | SEQ ID NO:1225 |
| TPTDPPPPYSAGPLLS | SEQ ID NO:1226 |
| TPTDPPPPYSAGPLLSV | SEQ ID NO:1227 |
| TPTDPPPPYSAGPLLSVP | SEQ ID NO:1228 |
| TPTDPPPPYSAGPLLSVPI | SEQ ID NO:1229 |
| TPTDPPPPYSAGPLLSVPIP | SEQ ID NO:1230 |
| DTPTDPPPPY | SEQ ID NO:1231 |
| DTPTDPPPPYS | SEQ ID NO:1232 |
| DTPTDPPPPYSA | SEQ ID NO:1233 |
| DTPTDPPPPYSAG | SEQ ID NO:1234 |
| DTPTDPPPPYSAGP | SEQ ID NO:1235 |
| DTPTDPPPPYSAGPL | SEQ ID NO:1236 |
| DTPTDPPPPYSAGPLL | SEQ ID NO:1237 |
| DTPTDPPPPYSAGPLLS | SEQ ID NO:1238 |
| DTPTDPPPPYSAGPLLSV | SEQ ID NO:1239 |
| DTPTDPPPPYSAGPLLSVP | SEQ ID NO:1240 |
| DTPTDPPPPYSAGPLLSVPI | SEQ ID NO:1241 |
| LDTPTDPPPPY | SEQ ID NO:1242 |
| LDTPTDPPPPYS | SEQ ID NO:1243 |
| LDTPTDPPPPYSA | SEQ ID NO:1244 |
| LDTPTDPPPPYSAG | SEQ ID NO:1245 |
| LDTPTDPPPPYSAGP | SEQ ID NO:1246 |
| LDTPTDPPPPYSAGPL | SEQ ID NO:1247 |
| LDTPTDPPPPYSAGPLL | SEQ ID NO:1248 |
| LDTPTDPPPPYSAGPLLS | SEQ ID NO:1249 |
| LDTPTDPPPPYSAGPLLSV | SEQ ID NO:1250 |
| LDTPTDPPPPYSAGPLLSVP | SEQ ID NO:1251 |
| DLDTPTDPPPPY | SEQ ID NO:1252 |
| DLDTPTDPPPPYS | SEQ ID NO:1253 |
| DLDTPTDPPPPYSA | SEQ ID NO:1254 |
| DLDTPTDPPPPYSAG | SEQ ID NO:1255 |
| DLDTPTDPPPPYSAGP | SEQ ID NO:1256 |
| DLDTPTDPPPPYSAGPL | SEQ ID NO:1257 |
| DLDTPTDPPPPYSAGPLL | SEQ ID NO:1258 |
| DLDTPTDPPPPYSAGPLLS | SEQ ID NO:1259 |
| DLDTPTDPPPPYSAGPLLSV | SEQ ID NO:1260 |
| IDLDTPTDPPPPY | SEQ ID NO:1261 |
| IDLDTPTDPPPPYS | SEQ ID NO:1262 |
| IDLDTPTDPPPPYSA | SEQ ID NO:1263 |
| IDLDTPTDPPPPYSAG | SEQ ID NO:1264 |
| IDLDTPTDPPPPYSAGP | SEQ ID NO:1265 |
| IDLDTPTDPPPPYSAGPL | SEQ ID NO:1266 |
| IDLDTPTDPPPPYSAGPLL | SEQ ID NO:1267 |
| LDLDTPTDPPPPYSAGPLLS | SEQ ID NO:1268 |
| VIDLDTPTDPPPPY | SEQ ID NO:1269 |
| VIDLDTPTDPPPPYS | SEQ ID NO:1270 |
| VIDLDTPTDPPPPYSA | SEQ ID NO:1271 |
| VIDLDTPTDPPPPYSAG | SEQ ID NO:1272 |
| VIDLDTPTDPPPPYSAGP | SEQ ID NO:1273 |
| VIDLDTPTDPPPPYSAGPL | SEQ ID NO:1274 |
| VIDLDTPTDPPPPYSAGPLL | SEQ ID NO:1275 |
| VVIDLDTPTDPPPPY | SEQ ID NO:1276 |
| VVIDLDTPTDPPPPYS | SEQ ID NO:1277 |
| VVIDLDTPTDPPPPYSA | SEQ ID NO:1278 |
| VVIDLDTPTDPPPPYSAG | SEQ ID NO:1279 |
| VVIDLDTPTDPPPPYSAGP | SEQ ID NO:1280 |
| VVIDLDTPTDPPPPYSAGPL | SEQ ID NO:1281 |
| FVVIDLDTPTDPPPPY | SEQ ID NO:1282 |
| FVVIDLDTPTDPPPPYS | SEQ ID NO:1283 |
| FVVIDLDTPTDPPPPYSA | SEQ ID NO:1284 |
| FVVIDLDTPTDPPPPYSAG | SEQ ID NO:1285 |
| FVVIDLDTPTDPPPPYSAGP | SEQ ID NO:1286 |
| PFVVIDLDTPTDPPPPY | SEQ ID NO:1287 |
| PFVVIDLDTPTDPPPPYS | SEQ ID NO:1288 |
| PFVVIDLDTPTDPPPPYSA | SEQ ID NO:1289 |
| PFVVIDLDTPTDPPPPYSAG | SEQ ID NO:1290 |
| GPFVVIDLDTPTDPPPPY | SEQ ID NO:1291 |
| GPFVVIDLDTPTDPPPPYS | SEQ ID NO:1292 |
| GPFVVIDLDTPTDPPPPYSA | SEQ ID NO:1293 |
| EGPFVVIDLDTPTDPPPPY | SEQ ID NO:1294 |
| EGPFVVIDLDTPTDPPPPYS | SEQ ID NO:1295 |
| NEGPFVVIDLDTPTDPPPPY | SEQ ID NO:1296 |

TABLE 9

PPPY Motif Containing Peptides from Human
Herpesvirus 7 Major Capsid Scaffold Protein
(GenBank Accession No. AAC40768)

| | |
|---|---|
| PPPYWYPS | SEQ ID NO:1297 |
| PPPYWYPSM | SEQ ID NO:1298 |
| PPPYWYPSMP | SEQ ID NO:1299 |
| PPPYWYPSMPG | SEQ ID NO:1300 |
| PPPYWYPSMPGF | SEQ ID NO:1301 |
| PPPYWYPSMPGFN | SEQ ID NO:1302 |
| PPPYWYPSMPGFNY | SEQ ID NO:1303 |
| PPPYWYPSMPGFNYK | SEQ ID NO:1304 |
| PPPYWYPSMPGFNYKS | SEQ ID NO:1305 |
| PPPYWYPSMPGFNYKSR | SEQ ID NO:1306 |
| PPPYWYPSMPGFNYKSRG | SEQ ID NO:1307 |
| PPPYWYPSMPGFNYKSRGS | SEQ ID NO:1308 |
| PPPYWYPSMPGFNYKSRGSQ | SEQ ID NO:1309 |
| IPPPYWYP | SEQ ID NO:1310 |
| IPPPYWYPS | SEQ ID NO:1311 |
| IPPPYWYPSM | SEQ ID NO:1312 |
| IPPPYWYPSMP | SEQ ID NO:1313 |
| IPPPYWYPSMPG | SEQ ID NO:1314 |
| IPPPYWYPSMPGF | SEQ ID NO:1315 |
| IPPPYWYPSMPGFN | SEQ ID NO:1316 |
| IPPPYWYPSMPGFNY | SEQ ID NO:1317 |
| IPPPYWYPSMPGFNYK | SEQ ID NO:1318 |
| IPPPYWYPSMPGFNYKS | SEQ ID NO:1319 |
| IPPPYWYPSMPGFNYKSR | SEQ ID NO:1320 |
| IPPPYWYPSMPGFNYKSRG | SEQ ID NO:1321 |
| IPPPYWYPSMPGFNYKSRGS | SEQ ID NO:1322 |
| HIPPPYWY | SEQ ID NO:1323 |
| HIPPPYWYP | SEQ ID NO:1324 |
| HIPPPYWYPS | SEQ ID NO:1325 |
| HIPPPYWYPSM | SEQ ID NO:1326 |
| HIPPPYWYPSMP | SEQ ID NO:1327 |
| HIPPPYWYPSMPG | SEQ ID NO:1328 |
| HIPPPYWYPSMPGF | SEQ ID NO:1329 |
| HIPPPYWYPSMPGFN | SEQ ID NO:1330 |
| HIPPPYWYPSMPGFNY | SEQ ID NO:1331 |
| HIPPPYWYPSMPGFNYK | SEQ ID NO:1332 |
| HIPPPYWYPSMPGFNYKS | SEQ ID NO:1333 |
| HIPPPYWYPSMPGFNYKSR | SEQ ID NO:1334 |
| HIPPPYWYPSMPGFNYKSRG | SEQ ID NO:1335 |
| YHIPPPYW | SEQ ID NO:1336 |
| YHIPPPYWY | SEQ ID NO:1337 |
| YHIPPPYWYP | SEQ ID NO:1338 |
| YHIPPPYWYPS | SEQ ID NO:1339 |
| YHIPPPYWYPSM | SEQ ID NO:1340 |
| YHIPPPYWYPSMP | SEQ ID NO:1341 |
| YHIPPPYWYPSMPG | SEQ ID NO:1342 |
| YHIPPPYWYPSMPGF | SEQ ID NO:1343 |
| YHIPPPYWYPSMPGFN | SEQ ID NO:1344 |
| YHIPPPYWYPSMPGFNY | SEQ ID NO:1345 |
| YHIPPPYWYPSMPGFNYK | SEQ ID NO:1346 |
| YHIPPPYWYPSMPGFNYKS | SEQ ID NO:1347 |
| YHIPPPYWYPSMPGFNYKSR | SEQ ID NO:1348 |
| NYHIPPPY | SEQ ID NO:1349 |
| NYHIPPPYW | SEQ ID NO:1350 |
| NYHIPPPYWY | SEQ ID NO:1351 |
| NYHIPPPYWYP | SEQ ID NO:1352 |
| NYHIPPPYWYPS | SEQ ID NO:1353 |
| NYHIPPPYWYPSM | SEQ ID NO:1354 |
| NYHIPPPYWYPSMP | SEQ ID NO:1355 |
| NYHIPPPYWYPSMPG | SEQ ID NO:1356 |
| NYHIPPPYWYPSMPGF | SEQ ID NO:1357 |
| NYHIPPPYWYPSMPGFN | SEQ ID NO:1358 |
| NYHIPPPYWYPSMPGFNY | SEQ ID NO:1359 |
| NYHIPPPYWYPSMPGFNYK | SEQ ID NO:1360 |
| NYHIPPPYWYPSMPGFNYKS | SEQ ID NO:1361 |
| MNYHIPPPY | SEQ ID NO:1362 |
| MNYHIPPPYW | SEQ ID NO:1363 |
| MNYHIPPPYWY | SEQ ID NO:1364 |
| MNYHIPPPYWYP | SEQ ID NO:1365 |
| MNYHIPPPYWYPS | SEQ ID NO:1366 |
| MNYHIPPPYWYPSM | SEQ ID NO:1367 |
| MNYHIPPPYWYPSMP | SEQ ID NO:1368 |
| MNYHIPPPYWYPSMPG | SEQ ID NO:1369 |
| MNYHIPPPYWYPSMPGF | SEQ ID NO:1370 |
| MNYHIPPPYWYPSMPGFN | SEQ ID NO:1371 |
| MNYHIPPPYWYPSMPGFNY | SEQ ID NO:1372 |
| MNYHIPPPYWYPSMPGFNYK | SEQ ID NO:1373 |
| RMNYHIPPPY | SEQ ID NO:1374 |
| RMNYHIPPPYW | SEQ ID NO:1375 |
| RMNYHIPPPYWY | SEQ ID NO:1376 |
| RMNYHIPPPYWYP | SEQ ID NO:1377 |
| RMNYHIPPPYWYPS | SEQ ID NO:1378 |
| RMNYHIPPPYWYPSM | SEQ ID NO:1379 |
| RMNYHIPPPYWYPSMP | SEQ ID NO:1380 |
| RMNYHIPPPYWYPSMPG | SEQ ID NO:1381 |
| RMNYHIPPPYWYPSMPGF | SEQ ID NO:1382 |
| RMNYHIPPPYWYPSMPGFN | SEQ ID NO:1383 |
| RMNYHIPPPYWYPSMPGFNY | SEQ ID NO:1384 |
| NRMNYHIPPPY | SEQ ID NO:1385 |
| NRMNYHIPPPYW | SEQ ID NO:1386 |
| NRMNYHIPPPYWY | SEQ ID NO:1387 |
| NRMNYHIPPPYWYP | SEQ ID NO:1388 |
| NRMNYHIPPPYWYPS | SEQ ID NO:1389 |
| NRMNYHIPPPYWYPSM | SEQ ID NO:1390 |
| NRMNYHIPPPYWYPSMP | SEQ ID NO:1391 |
| NRMNYHIPPPYWYPSMPG | SEQ ID NO:1392 |
| NRMNYHIPPPYWYPSMPGF | SEQ ID NO:1393 |
| NRMNYHIPPPYWYPSMPGFN | SEQ ID NO:1394 |
| GNRMNYHIPPPY | SEQ ID NO:1395 |
| GNRMNYHIPPPYW | SEQ ID NO:1396 |
| GNRMNYHIPPPYWY | SEQ ID NO:1397 |
| GNRMNYHIPPPYWYP | SEQ ID NO:1398 |
| GNRMNYHIPPPYWYPS | SEQ ID NO:1399 |
| GNRMNYHIPPPYWYPSM | SEQ ID NO:1400 |
| GNRMNYHIPPPYWYPSMP | SEQ ID NO:1401 |
| GNRMNYHIPPPYWYPSMPG | SEQ ID NO:1402 |
| GNRMNYHIPPPYWYPSMLPGF | SEQ ID NO:1403 |
| YGNRMNYHIPPPY | SEQ ID NO:1404 |
| YGNRMNYHIPPPYW | SEQ ID NO:1405 |
| YGNRMNYHIPPPYWY | SEQ ID NO:1406 |
| YGNRMNYHIPPPYWYP | SEQ ID NO:1407 |
| YGNRMNYHIPPPYWYPS | SEQ ID NO:1408 |
| YGNRMNYHIPPPYWYPSM | SEQ ID NO:1409 |
| YGNRMNYHIPPPYWYPSMP | SEQ ID NO:1410 |
| YGNRMNYHIPPPYWYPSMPG | SEQ ID NO:1411 |
| DYGNRMNYHIPPPY | SEQ ID NO:1412 |
| DYGNRMNYHIPPPYW | SEQ ID NO:1413 |
| DYGNRMNYHIPPPYWY | SEQ ID NO:1414 |
| DYGNRMNYHIPPPYWYP | SEQ ID NO:1415 |
| DYGNRMNYHIPPPYWYPS | SEQ ID NO:1416 |
| DYGNRMNYHIPPPYWYPSM | SEQ ID NO:1417 |
| DYGNRMNYHIPPPYWYPSMP | SEQ ID NO:1418 |
| MDYGNRMNYHIPPPY | SEQ ID NO:1419 |
| MDYGNRMNYHIPPPYW | SEQ ID NO:1420 |
| MDYGNRMNYHIPPPYWY | SEQ ID NO:1421 |
| MDYGNRMNYHIPPPYWYP | SEQ ID NO:1422 |
| MDYGNRMNYHIPPPYWYPS | SEQ ID NO:1423 |
| MDYGNRMNYHIPPPYWYPSM | SEQ ID NO:1424 |
| RMDYGNRMNYHIPPPY | SEQ ID NO:1425 |
| RMDYGNRMNYHIPPPYW | SEQ ID NO:1426 |
| RMDYGNRMNYHIPPPYWY | SEQ ID NO:1427 |
| RMDYGNRMNYHIPPPYWYP | SEQ ID NO:1428 |
| RMDYGNRMNYHIPPPYWYPS | SEQ ID NO:1429 |
| LRMDYGNRMNYHIPPPY | SEQ ID NO:1430 |
| LRMDYGNRMNYHIPPPYW | SEQ ID NO:1431 |
| LRMDYGNRMNYHIPPPYWY | SEQ ID NO:1432 |
| LRMDYGNRMNYHIPPPYWYP | SEQ ID NO:1433 |
| SLRMDYGNRMNYHIPPPY | SEQ ID NO:1434 |
| SLRMDYGNRMNYHIPPPYW | SEQ ID NO:1435 |
| SLRMDYGNRMNYHIPPPYWY | SEQ ID NO:1436 |
| ESLRMDYGNRMNYHIPPPY | SEQ ID NO:1437 |
| ESLRMDYGNRMNYHIPPPYW | SEQ ID NO:1438 |
| PESLRMDYGNRMNYHIPPPY | SEQ ID NO:1439 |

TABLE 10

PPXY Motif Containing Peptides from Infectious
Pancreatic Necrosis Virus Structural Protein VP2
(GenBank Accession No. AAK18736)

| | |
|---|---|
| EVELPPPY | SEQ ID NO:1440 |
| EEVELPPPY | SEQ ID NO:1441 |
| YEEVELPPPY | SEQ ID NO:1442 |
| NYEEVELPPPY | SEQ ID NO:1443 |
| ANYEEVELPPPY | SEQ ID NO:1444 |
| SANYEEVELPPPY | SEQ ID NO:1445 |
| ESANYEEVELPPPY | SEQ ID NO:1446 |
| LESANYEEVELPPPY | SEQ ID NO:1447 |
| RLESANYEEVELPPPY | SEQ ID NO:1448 |
| NRLESANYEEVELPPPY | SEQ ID NO:1449 |
| KNRLESANYEEVELPPPY | SEQ ID NO:1450 |
| LKNRLESANYEEVELPPPY | SEQ ID NO:1451 |
| ALKNRLESANYEEVELPPPY | SEQ ID NO:1452 |

TABLE 11

PPXY Motif Containing Peptides from Lassa Virus
Z Protein
(GenBank Accession No. AAC05816)

| | |
|---|---|
| IRPPPYSP | SEQ ID NO:1453 |
| SIRPPPYS | SEQ ID NO:1454 |
| SIRPPPYSP | SEQ ID NO:1455 |
| DSIRPPPY | SEQ ID NO:1456 |
| DSIRPPPYS | SEQ ID NO:1457 |
| DSIRPPPYSP | SEQ ID NO:1458 |
| ADSIRPPPY | SEQ ID NO:1459 |
| ADSIRPPPYS | SEQ ID NO:1460 |
| ADSIRPPPYSP | SEQ ID NO:1461 |
| AADSIRPPPY | SEQ ID NO:1462 |
| AADSIRPPPYS | SEQ ID NO:1463 |
| AADSIRPPPYSP | SEQ ID NO:1464 |
| GAADSIRPPPY | SEQ ID NO:1465 |
| GAADSIRPPPYS | SEQ ID NO:1466 |
| GAADSIRPPPYSP | SEQ ID NO:1467 |
| TGAADSIRPPPY | SEQ ID NO:1468 |
| TGAADSIRPPPYS | SEQ ID NO:1469 |
| TGAADSIRPPPYSP | SEQ ID NO:1470 |
| PTGAADSIRPPPY | SEQ ID NO:1471 |
| PTGAADSIRPPPYS | SEQ ID NO:1472 |
| PTGAADSIRPPPYSP | SEQ ID NO:1473 |
| PPTGAADSIRPPPY | SEQ ID NO:1474 |
| PPTGAADSIRPPPYS | SEQ ID NO:1475 |
| PPTGAADSIRPPPYSP | SEQ ID NO:1476 |
| APPTGAADSIRPPPY | SEQ ID NO:1477 |
| APPTGAADSIRPPPYS | SEQ ID NO:1478 |
| APPTGAADSIRPPPYSP | SEQ ID NO:1479 |
| TAPPTGAADSIRPPPY | SEQ ID NO:1480 |
| TAPPTGAADSIRPPPYS | SEQ ID NO:1481 |
| TAPPTGAADSIRPPPYSP | SEQ ID NO:1482 |
| PTAPPTGAADSIRPPPY | SEQ ID NO:1483 |
| PTAPPTGAADSIRPPPYS | SEQ ID NO:1484 |
| PTAPPTGAADSIRPPPYSP | SEQ ID NO:1485 |
| APTAPPTGAADSIRPPPY | SEQ ID NO:1486 |
| APTAPPTGAADSIRPPPYS | SEQ ID NO:1487 |
| APTAPPTGAADSIRPPPYSP | SEQ ID NO:1488 |
| AAPTAPPTGAADSIRPPPY | SEQ ID NO:1489 |
| AAPTAPPTGAADSIRPPPYS | SEQ ID NO:1490 |
| SAAPTAPPTGAADSIRPPPY | SEQ ID NO:1491 |

TABLE 12
PPPY Motif Containing Peptides from Lymphocytic
Choriomeningitis Virus Ring Finger Protein

(GenBank Accession No. CAA10342)

| | |
|---|---|
| SPPPPYEE | SEQ ID NO:1492 |
| PSPPPPYE | SEQ ID NO:1493 |
| PSPPPPYEE | SEQ ID NO:1494 |
| APSPPPPY | SEQ ID NO:1495 |
| APSPPPPYE | SEQ ID NO:1496 |
| APSPPPPYEE | SEQ ID NO:1497 |
| TAPSPPPPY | SEQ ID NO:1498 |
| TAPSPPPPYE | SEQ ID NO:1499 |
| TAPSPPPPYEE | SEQ ID NO:1500 |
| STAPSPPPPY | SEQ ID NO:1501 |
| STAPSPPPPYE | SEQ ID NO:1502 |
| STAPSPPPPYEE | SEQ ID NO:1503 |
| ISTAPSPPPPY | SEQ ID NO:1504 |
| ISTAPSPPPPYE | SEQ ID NO:1505 |
| ISTAPSPPPPYEE | SEQ ID NO:1506 |
| KISTAPSPPPPY | SEQ ID NO:1507 |
| KISTAPSPPPPYE | SEQ ID NO:1508 |
| KISTAPSPPPPYEE | SEQ ID NO:1509 |
| LKISTAPSPPPPY | SEQ ID NO:1510 |
| LKISTAPSPPPPYE | SEQ ID NO:1511 |
| LKISTAPSPPPPYEE | SEQ ID NO:1512 |
| KLKISTAPSPPPPY | SEQ ID NO:1513 |
| KLKISTAPSPPPPYE | SEQ ID NO:1514 |
| KLKISTAPSPPPPYEE | SEQ ID NO:1515 |
| TKLKISTAPSPPPPY | SEQ ID NO:1516 |
| TKLKISTAPSPPPPYE | SEQ ID NO:1517 |
| TKLKISTAPSPPPPYEE | SEQ ID NO:1518 |
| PTKLKISTAPSPPPPY | SEQ ID NO:1519 |
| PTKLKISTAPSPPPPYE | SEQ ID NO:1520 |
| PTKLKISTAPSPPPPYEE | SEQ ID NO:1521 |
| LPTKLKISTAPSPPPPY | SEQ ID NO:1522 |
| LPTKLKISTAPSPPPPYE | SEQ ID NO:1523 |
| LPTKLKISTAPSPPPPYEE | SEQ ID NO:1524 |
| PLPTKLKISTAPSPPPPY | SEQ ID NO:1525 |
| PLPTKLKISTAPSPPPPYE | SEQ ID NO:1526 |
| PLPTKLKISTAPSPPPPYEE | SEQ ID NO:1527 |
| CPLPTKLKISTAPSPPPPY | SEQ ID NO:1528 |
| CPLPTKLKISTAPSPPPPYE | SEQ ID NO:1529 |
| KCPLPTKLKISTAPSPPPPY | SEQ ID NO:1530 |

TABLE 13
PPXY Motif Containing Peptides from TT Virus ORF2
(GenBank Accession No. BAB19319)

| | |
|---|---|
| PPPYRSEP | SEQ ID NO:1531 |
| PPPYRSEPH | SEQ ID NO:1532 |
| PPPYRSEPHT | SEQ ID NO:1533 |
| PPPYRSEPHTE | SEQ ID NO:1534 |
| PPPYRSEPHTEH | SEQ ID NO:1535 |
| PPPYRSEPHTEHS | SEQ ID NO:1536 |
| PPPYRSEPHTEHSR | SEQ ID NO:1537 |
| PPPYRSEPHTEHSRP | SEQ ID NO:1538 |
| PPPYRSEPHTEHSRPP | SEQ ID NO:1539 |
| PPPYRSEPHTEHSRPPP | SEQ ID NO:1540 |
| PPPYRSEPHTEHSRPPPP | SEQ ID NO:1541 |
| PPPYRSEPHTEHSRPPPPK | SEQ ID NO:1542 |
| PPPYRSEPHTEHSRPPPPKK | SEQ ID NO:1543 |
| GPPPYRSE | SEQ ID NO:1544 |
| GPPPYRSEP | SEQ ID NO:1545 |
| GPPPYRSEPH | SEQ ID NO:1546 |
| GPPPYRSEPHT | SEQ ID NO:1547 |
| GPPPYRSEPHTE | SEQ ID NO:1548 |
| GPPPYRSEPHTEH | SEQ ID NO:1549 |
| GPPPYRSEPHTEHS | SEQ ID NO:1550 |
| GPPPYRSEPHTEHSR | SEQ ID NO:1551 |
| GPPPYRSEPHTEHSRP | SEQ ID NO:1552 |
| GPPPYRSEPHTEHSRPP | SEQ ID NO:1553 |
| GPPPYRSEPHTEHSRPPP | SEQ ID NO:1554 |
| GPPPYRSEPHTEHSRPPPP | SEQ ID NO:1555 |
| GPPPYRSEPHTEHSRPPPPK | SEQ ID NO:1556 |
| QGPPPYRS | SEQ ID NO:1557 |
| QGPPPYRSE | SEQ ID NO:1558 |
| QGPPPYRSEP | SEQ ID NO:1559 |
| QGPPPYRSEPH | SEQ ID NO:1560 |
| QGPPPYRSEPHT | SEQ ID NO:1561 |
| QGPPPYRSEPHTE | SEQ ID NO:1562 |
| QGPPPYRSEPHTEH | SEQ ID NO:1563 |
| QGPPPYRSEPHTEHS | SEQ ID NO:1564 |
| QGPPPYRSEPHTEHSR | SEQ ID NO:1565 |
| QGPPPYRSEPHTEHSRP | SEQ ID NO:1566 |
| QGPPPYRSEPHTEHSRPP | SEQ ID NO:1567 |
| QGPPPYRSEPHTEHSRPPP | SEQ ID NO:1568 |
| QGPPPYRSEPHTEHSRPPPP | SEQ ID NO:1569 |
| PQGPPPYR | SEQ ID NO:1570 |
| PQGPPPYRS | SEQ ID NO:1571 |
| PQGPPPYRSE | SEQ ID NO:1572 |
| PQGPPPYRSEP | SEQ ID NO:1573 |
| PQGPPPYRSEPH | SEQ ID NO:1574 |
| PQGPPPYRSEPHT | SEQ ID NO:1575 |
| PQGPPPYRSEPHTE | SEQ ID NO:1576 |
| PQGPPPYRSEPHTEH | SEQ ID NO:1577 |
| PQGPPPYRSEPHTEHS | SEQ ID NO:1578 |
| PQGPPPYRSEPHTEHSR | SEQ ID NO:1579 |
| PQGPPPYRSEPHTEHSRP | SEQ ID NO:1580 |
| PQGPPPYRSEPHTEHSRPP | SEQ ID NO:1581 |
| PQGPPPYRSEPHTEHSRPPP | SEQ ID NO:1582 |
| WPQGPPPY | SEQ ID NO:1583 |
| WPQGPPPYR | SEQ ID NO:1584 |
| WPQGPPPYRS | SEQ ID NO:1585 |
| WPQGPPPYRSE | SEQ ID NO:1586 |
| WPQGPPPYRSEP | SEQ ID NO:1587 |
| WPQGPPPYRSEPH | SEQ ID NO:1588 |
| WPQGPPPYRSEPHT | SEQ ID NO:1589 |
| WPQGPPPYRSEPHTE | SEQ ID NO:1590 |
| WPQGPPPYRSEPHTEH | SEQ ID NO:1591 |
| WPQGPPPYRSEPHTEHS | SEQ ID NO:1592 |
| WPQGPPPYRSEPHTEHSR | SEQ ID NO:1593 |
| WPQGPPPYRSEPHTEHSRP | SEQ ID NO:1594 |
| WPQGPPPYRSEPHTEHSRPP | SEQ ID NO:1595 |
| YWPQGPPPY | SEQ ID NO:1596 |
| YWPQGPPPYR | SEQ ID NO:1597 |
| YWPQGPPPYRS | SEQ ID NO:1598 |
| YWPQGPPPYRSE | SEQ ID NO:1599 |
| YWPQGPPPYRSEP | SEQ ID NO:1600 |
| YWPQGPPPYRSEPH | SEQ ID NO:1601 |
| YWPQGPPPYRSEPHT | SEQ ID NO:1602 |
| YWPQGPPPYRSEPHTE | SEQ ID NO:1603 |
| YWPQGPPPYRSEPHTEH | SEQ ID NO:1604 |
| YWPQGPPPYRSEPHTEHS | SEQ ID NO:1605 |
| YWPQGPPPYRSEPHTEHSR | SEQ ID NO:1606 |
| YWPQGPPPYRSEPHTEHSRP | SEQ ID NO:1607 |
| GYWPQGPPPY | SEQ ID NO:1608 |
| GYWPQGPPPYR | SEQ ID NO:1609 |
| GYWPQGPPPYRS | SEQ ID NO:1610 |
| GYWPQGPPPYRSE | SEQ ID NO:1611 |
| GYWPQGPPPYRSEP | SEQ ID NO:1612 |
| GYWPQGPPPYRSEPH | SEQ ID NO:1613 |
| GYWPQGPPPYRSEPHT | SEQ ID NO:1614 |
| GYWPQGPPPYRSEPHTE | SEQ ID NO:1615 |
| GYWPQGPPPYRSEPHTEH | SEQ ID NO:1616 |
| GYWPQGPPPYRSEPHTEHS | SEQ ID NO:1617 |
| GYWPQGPPPYRSEPHTEHSR | SEQ ID NO:1618 |
| RGYWPQGPPPY | SEQ ID NO:1619 |
| RGYWPQGPPPYR | SEQ ID NO:1620 |
| RGYWPQGPPPYRS | SEQ ID NO:1621 |
| RGYWPQGPPPYRSE | SEQ ID NO:1622 |
| RGYWPQGPPPYRSEP | SEQ ID NO:1623 |
| RGYWPQGPPPYRSEPH | SEQ ID NO:1624 |
| RGYWPQGPPPYRSEPHT | SEQ ID NO:1625 |
| RGYWPQGPPPYRSEPHTE | SEQ ID NO:1626 |
| RGYWPQGPPPYRSEPHTEH | SEQ ID NO:1627 |
| RGYWPQGPPPYRSEPHTEHS | SEQ ID NO:1628 |
| TRGYWPQGPPPY | SEQ ID NO:1629 |
| TRGYWPQGPPPYR | SEQ ID NO:1630 |
| TRGYWPQGPPPYRS | SEQ ID NO:1631 |
| TRGYWPQGPPPYRSE | SEQ ID NO:1632 |
| TRGYWPQGPPPYRSEP | SEQ ID NO:1633 |
| TRGYWPQGPPPYRSEPH | SEQ ID NO:1634 |
| TRGYWPQGPPPYRSEPHT | SEQ ID NO:1635 |
| TRGYWPQGPPPYRSEPHTE | SEQ ID NO:1636 |
| TRGYWPQGPPPYRSEPHTEH | SEQ ID NO:1637 |
| QTRGYWPQGPPPY | SEQ ID NO:1638 |
| QTROYWPQGPPPYR | SEQ ID NO:1639 |
| QTRGYWPQGPPPYRS | SEQ ID NO:1640 |
| QTRGYWPQGPPPYRSE | SEQ ID NO:1641 |
| QTRGYWPQGPPPYRSEP | SEQ ID NO:1642 |
| QTRGYWPQGPPPYRSEPH | SEQ ID NO:1643 |
| QTRGYWPQGPPPYRSEPHT | SEQ ID NO:1644 |
| QTRGYWPQGPPPYRSEPHTE | SEQ ID NO:1645 |
| LQTRGYWPQGPPPY | SEQ ID NO:1646 |
| LQTRGYWPQGPPPYR | SEQ ID NO:1647 |
| LQTRGYWPQGPPPYRS | SEQ ID NO:1648 |
| LQTRGYWPQGPPPYRSE | SEQ ID NO:1649 |
| LQTRGYWPQGPPPYRSEP | SEQ ID NO:1650 |
| LQTRGYWPQGPPPYRSEPH | SEQ ID NO:1651 |
| LQTRGYWPQGPPPYRSEPHT | SEQ ID NO:1652 |
| ILQTRGYWPQGPPPY | SEQ ID NO:1653 |
| ILQTRGYWPQGPPPYR | SEQ ID NO:1654 |
| ILQTRGYWPQGPPPYRS | SEQ ID NO:1655 |
| ILQTRGYWPQGPPPYRSE | SEQ ID NO:1656 |
| ILQTRGYWPQGPPPYRSEP | SEQ ID NO:1657 |
| ILQTRGYWPQGPPPYRSEPH | SEQ ID NO:1658 |
| NILQTRGYWPQGPPPY | SEQ ID NO:1659 |
| NILQTRGYWPQGPPPYR | SEQ ID NO:1660 |
| NILQTRGYWPQGPPPYRS | SEQ ID NO:1661 |
| NILQTRGYWPQGPPPYRSE | SEQ ID NO:1662 |
| NILQTRGYWPQGPPPYRSEP | SEQ ID NO:1663 |
| RNILQTRGYWPQGPPPY | SEQ ID NO:1664 |
| RNILQTRGYWPQGPPPYR | SEQ ID NO:1665 |
| RNILQTRGYWPQGPPPYRS | SEQ ID NO:1666 |
| RNILQTRGYWPQGPPPYRSE | SEQ ID NO:1667 |
| LRNILQTRGYWPQGPPPY | SEQ ID NO:1668 |
| LRNILQTRGYWPQGPPPYR | SEQ ID NO:1669 |
| LRNILQTRGYWPQGPPPYRS | SEQ ID NO:1670 |
| HLRNILQTRGYWPQGPPPY | SEQ ID NO:1671 |
| HLRNILQTRGYWPQGPPPYR | SEQ ID NO:1672 |
| DHLRNILQTRGYWPQGPPPY | SEQ ID NO:1673 |

What is claimed is:

1. A composition comprising a peptide associated with a transporter that is capable of increasing the uptake of said peptide by a mammalian cell,
wherein said peptide includes an amino acid sequence motif PPXY and is capable of binding a type I WW-domain of the Nedd4 protein, wherein X is an amino acid.

2. The composition according to claim 1, wherein X is selected from the group consisting of proline (P), alanine (A), glutamic acid (E), asparagine (N), and arginine (R).

3. The composition of claim 1, wherein said transporter is capable of increasing the uptake of said peptide by a mammalian cell by at least 100%.

4. The composition of claim 1, wherein said transporter is capable of increasing the uptake of said peptide by a mammalian cell by at least 300%.

5. The composition of claim 1, wherein said peptide is covalently linked to said transporter.

6. The composition of claim 5, wherein said transporter is selected from the group consisting of penetratins, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histidine oligomers, D-histidine oligomers, L-ornithine oligomers, D-ornithine oligomers, and HSV-1 structural protein VP22 and fragments thereof, and peptides having at least six contiguous amino acid residues that are L-arginine, D-arginine, L-lysine, D-lysine, L-histidine, D-histidine, L-ornithine, D-ornithine, or a combination thereof; and peptoid analogs thereof.

7. The composition according to claim 1, wherein said transporter is selected from the group consisting of liposomes, dendrimers, and siderophores.

8. The composition according to claim 1, wherein said peptide includes a contiguous amino acid sequence of at least 6 amino acid residues of a viral protein selected from the group consisting of matrix proteins of rhabdoviruses, matrix proteins of filoviruses, Rous Sarcoma virus GAG protein, hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, TT virus ORF2 protein, and wherein said contiguous amino acid sequence encompasses the PPXY motif of said viral protein.

9. The composition according to claim 1, wherein said peptide includes a contiguous amino acid sequence of at least 6 amino acid residues of a viral protein selected from the group consisting of Ebola virus Matrix (EbVp40) protein, Rous Sarcoma virus GAG protein, Marburg virus matrix protein, VSV matrix protein, and Mason-Pfizer Monkey virus GAG protein, and wherein said contiguous amino acid sequence encompasses the PPXY motif of said viral protein.

10. A composition comprising a hybrid polypeptide, said hybrid polypeptide consists of a peptide covalently linked to a peptidic transporter that is capable of increasing the uptake of said peptide by a mammalian cell by at least 100%,
wherein said hybrid polypeptide consists of from about 8 to about 100 amino acid residues, and wherein said peptide comprises an amino acid sequence motif PPXY and is capable of binding a type I WW-domain of the Nedd4 protein, wherein X is an amino acid.

11. The composition according to claim 10, wherein said hybrid polypeptide consists of from about 9 to about 50 amino acid residues.

12. The composition according to claim 10, wherein said hybrid polypeptide consists of from about 12 to about 30 amino acid residues.

13. The composition according to claim 10, wherein X is selected from the group consisting of proline (P), alanine (A), glutamic acid (E), asparagine (N), and arginine (R).

14. The composition according to claim 10, wherein said peptide includes a contiguous amino acid sequence of at least 6 amino acid residues of a viral protein selected from the group consisting of matrix proteins of rhabdoviruses, matrix proteins of filoviruses, Rous Sarcoma virus GAG protein, hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, TT virus ORF2 protein, and wherein said contiguous amino acid sequence encompasses the PPXY motif of said viral protein.

15. The composition according to claim 10, wherein said peptide includes a contiguous amino acid sequence of at least 6 amino acid residues of a viral protein selected from the group consisting of Ebola virus Matrix (EbVp40) protein, Rous Sarcoma virus GAG protein, Marburg virus matrix protein, VSV matrix protein, and Mason-Pfizer Monkey virus GAG protein, and wherein said contiguous amino acid sequence encompasses the PPXY motif of said viral protein.

16. The composition according to claim 10, wherein said peptide does not include a contiguous amino acid sequence of Ebola virus Matrix (EbVp40) protein that is sufficient to impart an ability to bind the UEV domain of the human Tsg101 protein.

17. The composition according to claim 10, wherein said transporter that is capable of increasing the uptake of said peptide by a mammalian cell by at least 300%.

18. The composition according to claim 10, wherein said transporter is selected from the group consisting of penetratins, l-Tat₄₉₋₅₇, retro-inverso isomers of l-Tat₄₉₋₅₇, L-arginine oligomers, L-lysine oligomers, HSV-1 structural protein VP22 and fragments thereof, and peptides consisting of at least six contiguous amino acid residues that are a combination of two or more of L-arginine, L-lysine and L-histidine.

19. The composition according to claim 11, wherein said peptide comprises an amino acid sequence selected from the group consisting of SEQ ID NOs:24-36, SEQ ID NOs:154-295, SEQ ID NOs:296-438, SEQ ID NOs:439-581, SEQ ID NOs:582-724, SEQ ID NOs:725-1010, SEQ ID NOs:1011-1296, SEQ ID NOs:1297-1439, SEQ ID NOs:1440-1452, SEQ ID NOs:1453-1491, SEQ ID NOs:1492-1530, and SEQ ID NOs:1531-1673.

20. The composition according to claim 10, wherein said hybrid polypeptide does not contain a terminal L-histidine oligomer.

21. A composition comprising a hybrid polypeptide, said hybrid polypeptide consists of a peptide covalently linked to a peptidic transporter that is capable of increasing the uptake of said peptide by a mammalian cell by at least 200%,
wherein said hybrid polypeptide consists of from about 10 to about 30 amino acid residues, and wherein said peptide comprises an amino acid sequence motif PPXY and is capable of binding a type I WW-domain of the Nedd4 protein, wherein X is an amino acid.

22. The composition of claim 21, wherein said hybrid polypeptide does not contain a terminal L-histidine oligomer of at least 6 histidine residues.

23. An isolated nucleic acid encoding the hybrid polypeptide according to claim 10.

24. An isolated nucleic acid encoding the hybrid polypeptide according to claim 11.

25. An isolated nucleic acid encoding the hybrid polypeptide according to claim 22.

26. A host cell comprising the isolated nucleic acid according to claim 23.

27. A host cell comprising the isolated nucleic acid according to claim 24.

28. A host cell comprising the isolated nucleic acid according to claim 25.

29. An isolated peptide consisting of a contiguous amino acid sequence of from 8 to about 30 amino acid residues of a viral protein selected from the group consisting of hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, and TT virus ORF2 protein, wherein said contiguous amino acid sequence encompasses the PPXY motif of said viral protein, and wherein said peptide is capable of binding a type I WW-domain of the Nedd4 protein.

30. The isolated peptide according to claim 29, wherein said isolated peptide consists of from 9 to about 20 amino acid residues.

31. The isolated peptide of claim 29, wherein said peptide comprises of an amino acid sequence selected from the group consisting of SEQ ID NOs:24-36, SEQ ID NOs:154-295, SEQ ID NOs:296-438, SEQ ID NOs:439-581, SEQ ID NOs:582-724, SEQ ID NOs:725-1010, SEQ ID NOs:1011-1296, SEQ ID NOs:1297-1439, SEQ ID NOs:1440-1452, SEQ ID NOs:1453-1491, SEQ ID NOs:1492-1530, and SEQ ID NOs:1531-1673.

32. An isolated nucleic acid encoding the isolated peptide according to claim 29.

33. An isolated nucleic acid encoding the isolated peptide according to claim 30.

34. An isolated nucleic acid encoding the isolated peptide according to claim 31.

35. A method for treating an infection caused by a virus selected from the group consisting of hepatitis B virus and human herpesvirus 1, said method comprising:
introducing into a patient in need of such treatment a peptide consisting of from 8 to about 30 amino acid residues and having an amino acid sequence motif PPXY, wherein X is an amino acid, and wherein said peptide is capable of binding a type I WW-domain of the Nedd4 protein.

36. The method of claim 35, wherein said introducing step comprises administering to the cells a nucleic acid encoding said peptide.

37. The method of claim 35, wherein X is selected from the group consisting of proline (P), alanine (A), glutamic acid (E), asparagine (N), and arginine (R).

38. The method of claim 35, wherein said peptide includes a contiguous amino acid sequence of at least 8 residues of a viral protein selected from the group consisting of matrix proteins of rhabdoviruses, matrix proteins of filoviruses, Rous Sarcoma virus GAG protein, Mason-Pfizer Monkey virus GAG protein, hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, TT virus ORF2 protein, and wherein said contiguous amino acid sequence encompasses the PPXY motif of said viral protein.

39. A method for treating an infection caused by a virus selected from the group consisting of hepatitis B virus and human herpesvirus 1, said method comprising:
administering to a patient in need of such treatment a composition comprising a peptide associated with a transporter that is capable of increasing the uptake of said peptide by a mammalian cell,
wherein said peptide includes an amino acid sequence motif PPXY and is capable of binding a type I WW-domain of the Nedd4 protein, wherein X is an amino acid.

40. The method according to claim 39, wherein X is selected from the group consisting of proline (P), alanine (A), glutamic acid (E), asparagine (N), and arginine (R).

41. The method according to claim 39, wherein said transporter is capable of increasing the uptake of said peptide by a mammalian cell by at least 100%.

42. The method according to claim 39, wherein said transporter is capable of increasing the uptake of said peptide by a mammalian cell by at least 300%.

43. The method according to claim 39, wherein said peptide is covalently linked to said transporter.

44. The method according to claim 43, wherein said transporter is selected from the group consisting of penetrating, l-Tat₄₉₋₅₇, d-Tat₄₉₋₅₇, retro-inverso isomers of l- or d-Tat₄₉₋₅₇, L-arginine oligomers, D-arginine oligomers, L-lysine oligomers, D-lysine oligomers, L-histidine oligomers, D-histidine oligomers, L-ornithine oligomers, D-ornithine oligomers, and HSV-1 structural protein VP22 and fragments thereof, and peptides having at least six contiguous amino acid residues that are L-arginine, D-arginine, L-lysine, D-lysine, L-histidine, D-histidine, L-ornithine, D-ornithine, or a combination thereof; and peptoid analogs thereof.

45. The method according to claim 39, wherein said transporter is selected from the group consisting of liposomes, dendrimers, and siderophores.

46. The method according to claim 39, wherein said peptide includes a contiguous amino acid sequence of at least 6 amino acid residues of a viral protein selected from the group consisting of matrix proteins of rhabdoviruses, matrix proteins of filoviruses, Rous Sarcoma virus GAG protein, Mason-Pfizer Monkey virus GAG protein, hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, TT virus ORF2 protein, and wherein said contiguous amino acid sequence encompasses the PPXY motif of said viral protein.

47. The method according to claim 39, wherein said peptide includes a contiguous amino acid sequence of at least 6 amino acid residues of a viral protein selected from the group consisting of Ebola virus Matrix (EbVp40) protein, Rous Sarcoma virus GAG protein, Marburg virus matrix protein, VSV matrix protein, and Mason-Pfizer Monkey virus GAG protein, and wherein said contiguous amino acid sequence encompasses the PPXY motif of said viral protein.

48. A method for treating an infection caused by a virus selected from the group consisting of hepatitis B virus and human herpesvirus 1, said method comprising:
administering to a patient in need of such treatment a hybrid polypeptide, said hybrid polypeptide consists of a peptide covalently linked to a peptidic transporter that is capable of increasing the uptake of said peptide by a mammalian cell by at least 100%,
wherein said hybrid polypeptide consists of from about 8 to about 100 amino acid residues, and wherein said peptide comprises an amino acid sequence motif PPXY and is capable of binding a type I WW-domain of the Nedd4 protein, wherein X is an amino acid.

49. The method according to claim 48, wherein said hybrid polypeptide consists of from about 9 to about 50 amino acid residues.

50. The method according to claim 48, wherein said hybrid polypeptide consists of from about 12 to about 30 amino acid residues.

51. The method according to claim 48, wherein X is selected from the group consisting of proline (P), alanine (A), glutamic acid (E), asparagine (N), and arginine (R).

52. The method according to claim 48, wherein said peptide includes a contiguous amino acid sequence of at least 6 amino acid residues of a viral protein selected from the group consisting of matrix proteins of rhabdoviruses, matrix proteins of filoviruses, Rous Sarcoma virus GAG protein, Mason-Pfizer Monkey virus GAG protein, hepatitis B virus core antigen, human herpesvirus 4 latent membrane protein 2A, human herpesvirus 1 UL56 protein, human herpesvirus 7 major capsid scaffold protein, infectious pancreatic necrosis virus VP2 protein, Lassa virus Z protein, lymphocytic choriomeningitis virus ringer finger protein, TT virus ORF2 protein, and wherein said contiguous amino acid sequence encompasses the PPXY motif of said viral protein.

53. The method according to claim 48, wherein said peptide includes a contiguous amino acid sequence of at least 6 amino acid residues of a viral protein selected from the group consisting of Ebola virus Matrix (EbVp40) protein, Rous Sarcoma virus GAG protein, Marburg virus matrix protein, VSV matrix protein, and Mason-Pfizer Monkey virus GAG protein, and wherein said contiguous amino acid sequence encompasses the PPXY motif of said viral protein.

54. The method according to claim 48, wherein said peptide does not include a contiguous amino acid sequence of Ebola virus Matrix (EbVp40) protein that is sufficient to impart an ability to bind the UEV domain of the human Tsg101 protein.

55. The method according to claim 48, wherein said transporter is capable of increasing the uptake of said peptide by a mammalian cell by at least 300%.

56. The method according to claim 48, wherein said transporter is selected from the group consisting of penetratins, l-Tat₄₉₋₅₇, retro-inverso isomers of l-Tat₄₉₋₅₇, L-arginine oligomers, L-lysine oligomers, HSV-1 structural protein VP22 and fragments thereof, and peptides consisting of at least six contiguous amino acid residues that include two or more of the group consisting of L-arginine, L-lysine and L-histidine.

57. The method according to claim 48, wherein said peptide comprises an amino acid sequence selected from the group consisting of SEQ ID NOs:24-36, SEQ ID NOs:154-295, SEQ ID NOs:296-438, SEQ ID NOs:439-581, SEQ ID NOs:582-724, SEQ ID NOs:725-1010, SEQ ID NOs:1011-1296, SEQ ID NOs:1297-1439, SEQ ID NOs:1440-1452, SEQ ID NOs:1453-1491, SEQ ID NOs:1492-1530, and SEQ ID NOs:1531-1673.

58. The method according to claim 48, wherein said hybrid polypeptide does not contain a terminal L-histidine oligomer.

59. A method for treating an infection caused by a virus selected from the group consisting of hepatitis B virus and human herpesvirus 1, said method comprising:
administering to a patient in need of such treatment a composition comprising a hybrid polypeptide, said hybrid polypeptide consists of a peptide covalently linked to a peptidic transporter that is capable of increasing the uptake of said peptide by a mammalian cell by at least 200%,
wherein said hybrid polypeptide consists of from about 10 to about 30 amino acid residues, and wherein said peptide comprises an amino acid sequence motif PPXY and is capable of binding a type I WW-domain of the Nedd4 protein, wherein X is an amino acid.
